(19) United States
(12) Patent Application Publication
(10) Pub. No.: US 2021/0379033 A1
(43) Pub. Date: Dec. 9, 2021

(54) METHODS OF TREATING CANCER WITH ARYL HYDROCARBON RECEPTOR ANTAGONISTS

(71) Applicant: Magenta Therapeutics Inc., Cambridge, MA (US)

(72) Inventor: Anthony BOITANO, Waban, MA (US)

(21) Appl. No.: 17/285,973

(22) PCT Filed: Oct. 17, 2019
(86) PCT No.: PCT/US2019/056774

§ 371 Date: Apr. 16, 2021

Related U.S. Application Data
(60) Provisional application No. 62/747,064, filed on Oct. 17, 2018.

(51) Int. Cl. A61K 31/444; A61K 31/4985

(52) U.S. Cl. CPC A61K 31/444; A61K 31/4985

(57) ABSTRACT

The disclosure relates to aryl hydrocarbon receptor antagonists, such as substituted imidazopyridines and imidazopyrazines, as well as methods of modulating aryl hydrocarbon receptor activity and treating various pathologies, such as cancer, by administration of these aryl hydrocarbon receptor antagonists. Additionally, the disclosure provides methods of synthesizing aryl hydrocarbon receptor antagonists, such as substituted imidazopyridines and imidazopyrazines, as well as compositions and kits containing aryl hydrocarbon receptor antagonists that can be used for the treatment of diseases and disorders.

### RELATED APPLICATION

This application claims priority to U.S. Application No. 62/747,064, filed Oct. 17, 2018, the entire contents of which are incorporated herein by reference.

### FIELD

The present disclosure relates to aryl hydrocarbon receptor antagonists, such as substituted imidazopyridines and imidazopyrazines useful, for example, for modulating the activity of an aryl hydrocarbon receptor, as well as methods of treating various pathologies, such as cancer, by administration of the aryl hydrocarbon receptor antagonists.

### BACKGROUND

Cancer remains one of the most deadly threats to human health. In the U.S., cancer affects nearly 1.3 million new patients each year, and is the second leading cause of death after heart disease, accounting for approximately 1 in 4 deaths. It is also predicted that cancer may surpass cardiovascular diseases as the number one cause of death within the next decade. Solid tumors are responsible for many of those deaths. Although there have been significant advances in the medical treatment of certain cancers, the overall 5-year survival rate for all cancers has improved only by about 10% in the past 20 years. Cancers, or malignant tumors, metastasize and grow rapidly in an uncontrolled manner, making timely detection and treatment extremely difficult.

There is currently a need for novel agents that modulate aryl hydrocarbon receptor activity. There is currently a need for novel agents for use in therapeutic compositions and methods thereof for inhibiting cancer cell proliferation and tumor cell invasion and metastasis, such as compounds that modulate aryl hydrocarbon receptor activity.

### SUMMARY

In some aspects, the present disclosure provides a method of modulating the activity of an aryl hydrocarbon receptor, comprising administering to a subject in need thereof an effective amount of a compound (e.g., an aryl hydrocarbon receptor antagonist) described herein.

In some aspects, the present disclosure provides a method of treating or preventing a disease or disorder, comprising administering to a subject in need thereof an effective amount of a compound (e.g., an aryl hydrocarbon receptor antagonist) described herein.

In some aspects, the present disclosure provides a compound (e.g., an aryl hydrocarbon receptor antagonist) described herein for use in modulating the activity of an aryl hydrocarbon receptor in a subject in need thereof.

In some aspects, the present disclosure provides a compound (e.g., an aryl hydrocarbon receptor antagonist) described herein for use in treating or preventing a disease or disorder in a subject in need thereof.

In some aspects, the present disclosure provides use of a compound (e.g., an aryl hydrocarbon receptor antagonist) described herein in the preparation or manufacture of a medicament for modulating the activity of an aryl hydrocarbon receptor in a subject in need thereof.

In some aspects, the present disclosure features use of a compound (e.g., an aryl hydrocarbon receptor antagonist) described herein in the preparation or manufacture of a medicament for treating or preventing a disease or disorder in a subject in need thereof.

In some aspects, the disclosure provides a pharmaceutical composition comprising a compound (e.g., aryl hydrocarbon receptor antagonist) described herein and a pharmaceutically acceptable carrier.

In some aspects, the disclosure provides a kit comprising a compound (e.g., aryl hydrocarbon receptor antagonist) described herein and a pharmaceutically acceptable carrier.

In some aspects, the present disclosure provides a pharmaceutical composition or kit described herein for use in modulating the activity of an aryl hydrocarbon receptor in a subject in need thereof.

In some aspects, the present disclosure provides a pharmaceutical composition or kit described herein for use in treating or preventing a disease or disorder in a subject in need thereof.

In some embodiments, the disease or disorder is characterized by the production of an aryl hydrocarbon receptor agonist.

In some embodiments, the disease or disorder is a cancer, a cancerous condition, or a tumor.

In some embodiments, the tumor is a solid tumor.

In some embodiments, the tumor is an invasive tumor.

In some embodiments, the cancer is a breast cancer, squamous cell cancer, lung cancer, a cancer of the peritoneum, a hepatocellular cancer, a gastric cancer, a pancreatic cancer, a glioblastoma, a cervical cancer, an ovarian cancer, a liver cancer, a bladder cancer, a hepatoma, a colon cancer, a colorectal cancer, an endometrial or uterine carcinoma, a salivary gland carcinoma, a kidney or renal cancer, a prostate cancer, a vulval cancer, a thyroid cancer, a head and neck cancer, a B-cell lymphoma, a chronic lymphocytic leukemia (CLL); an acute lymphoblastic leukemia (ALL), a Hairy cell leukemia, or a chronic myeloblastic leukemia.

In some embodiments, the cancer is a hematological cancer.

In some embodiments, the cancer is leukemia, lymphoma, multiple myeloma, or neuor blastoma. In some embodiments, the cancer is acute myeloid leukemia, acute lymphoid leukemia, chronic myeloid leukemia, chronic lymphoid leukemia, multiple myeloma, diffuse large B-cell lymphoma, or non-Hodgkin's lymphoma.

In some embodiments, one or more additional anti-cancer therapies is administered to the subject.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### BRIEF DESCRIPTION OF THE FIGURES

FIG. 1 is a graph demonstrating the effect of compound (5) and compound (16) on the aryl hydrocarbon receptor-driven expression of luciferase in the absence of the aryl hydrocarbon receptor agonist VAF347 in transiently transfected HepG2 cells in vitro. Experimental details for this experiment are reported in Example 3, below.

FIG. 2 is a graph demonstrating the effect of compound (5) and compound (16) on the aryl hydrocarbon receptor-driven expression of luciferase in the presence of the aryl hydrocarbon receptor agonist VAF347 in transiently transfected HepG2 cells in vitro. Experimental details for this experiment are reported in Example 3, below.

FIG. 3 sets forth a ¹H-NMR spectra of compound (24) in DMSO-d6.

FIG. 4 sets forth a ¹H-NMR spectra of an isolated compound (24) enantiomer peak in DMSO-d6.

FIG. 5 sets forth a ¹H-NMR spectra of an isolated compound (24) enantiomer peak in DMSO-d6.

FIG. 6 sets forth a ¹H-NMR spectra of compound (27) in chloroform-d.

FIG. 7 sets forth a ¹H-NMR spectra of compound (28) in chloroform-d.

FIG. 8 is a graph demonstrating endogeneous AHR antagonist activity. Experimental details for this experiment are reported in Example 7, below.

FIG. 9 is a graph demonstrating AHR antagonist activity in the presence of VAF347. Experimental details for this experiment are reported in Example 7, below.

### DETAILED DESCRIPTION

The compositions and methods described herein provide tools for treating pathologies, such as cancer, by administration of an aryl hydrocarbon receptor antagonist represented by formula ( ) or (II) described herein. For example, described herein are novel small molecule modulators of the aryl hydrocarbon receptor (AHR) and associated AHR signaling, as well as, methods of treating and inhibiting cancer growth and tumor cell invasion, and other hyperproliferative disorders.

### Definitions

Listed below are definitions of various terms used in this application. These definitions apply to terms as they are used throughout this specification and claims, unless otherwise limited in specific instances, either individually or as part of a larger group.

As used herein, the term “about” refers to a value that is within 10% above or below the value being described. For example, the term “about 5 nM” indicates a range of from 4.5 nM to 5.5 nM.

As used herein, the term “aryl hydrocarbon receptor (AHR) modulator” refers to an agent that causes or facilitates a qualitative or quantitative change, alteration, or modification in one or more processes, mechanisms, effects, responses, functions, activities or pathways mediated by the AHR receptor. Such changes mediated by an AHR modulator, such as an inhibitor or a non-constitutive agonist of the AHR described herein, can refer to a decrease or an increase in the activity or function of the AHR, such as a decrease in, inhibition of, or diversion of, constitutive activity of the AHR.

An “AHR antagonist” refers to an AHR inhibitor that does not provoke a biological response itself upon specifically binding to the AHR polypeptide or polynucleotide encoding the AHR, but blocks or dampens agonist-mediated or ligand-mediated responses, i.e., an AHR antagonist can bind but does not activate the AHR polypeptide or polynucleotide encoding the AHR, and the binding disrupts the interaction, displaces an AHR agonist, and/or inhibits the function of an AHR agonist. Thus, as used herein, an AHR antagonist does not function as an inducer of AHR activity when bound to the AHR, i.e., they function as pure AHR inhibitors.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) taken from a subject.

As used herein, the terms “subject” and “patient” refer to an organism, such as a human, that receives treatment for a particular disease or condition as described herein. The term subject or patient as used herein may refer to a mammal, a subject or patient therefore refers to, for example, dogs, cats, horses, cows, pigs, guinea pigs, and the like. For instance, a patient, such as a human patient, that is in need of an aryl hydrocarbon receptor antagonist may receive treatment that includes an aryl hydrocarbon receptor antagonist so as to treat a disease or disorder, such as a cancer, autoimmune disease, or metabolic disorder described herein. For instance, a patient, such as a human patient suffering from a disease or disorder, may receive treatment in the form of an aryl hydrocarbon receptor antagonist.

The term “cancer” includes, but is not limited to, the following cancers: epidermoid Oral: buccal cavity, lip, tongue, mouth, pharynx; Cardiac: sarcoma (angiosarcoma, fibrosarcoma, rhabdomyosarcoma, liposarcoma), myxoma, rhabdomyoma, fibroma, lipoma, and teratoma; Lung: bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Karposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal, rectum; Genitouinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma, biliary passages; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rthabdomyosarcoma), fallopian tubes (carcinoma), breast; Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma) hairy cell; lymphoid disorders; Skin: malignant melanoma, basal cell carcinoma, squamous cell carcinoma, Karposi's sarcoma, keratoacanthoma, moles dysplastic nevi, lipoma, angioma, dermatofibroma, keloids, psoriasis, Thyroid gland: papillary thyroid carcinoma, follicular thyroid carcinoma; medullary thyroid carcinoma, undifferentiated thyroid cancer, multiple endocrine neoplasia type 2A, multiple endocrine neoplasia type 2B, familial medullary thyroid cancer, pheochromocytoma, paraganglioma; and Adrenal glands: neuroblastoma. Thus, the term “cancerous cell” as provided herein, includes a cell afflicted by any one of the above-identified conditions.

As used herein, the phrase “disease” or “disorder” broadly refers to any disease, disorder, or condition that may be treated or cured by administering a compound or aryl hydrocarbon receptor antagonist as described herein to a patient. Diseases that may be treated by include, but are not limited to, sickle cell anemia, thalassemias, Fanconi anemia, aplastic anemia, Wiskott-Aldrich syndrome, ADA SCID, HIV/AIDS, metachromatic leukodystrophy, Diamond-Blackfan anemia, and Schwachman-Diamond syndrome. Additional diseases that may be treated as described herein include blood disorders (e.g., sickle cell anemia) and autoimmune disorders, such as scleroderma, multiple sclerosis, ulcerative colitis, and Chrohn's disease. Additional diseases that may be treated include cancer, such as a cancer described herein, including a malignancy, such as a neuroblastoma or a hematologic cancers, such as leukemia, lymphoma, and myeloma. For instance, the cancer may be acute myeloid leukemia, acute lymphoid leukemia, chronic myeloid leukemia, chronic lymphoid leukemia, multiple myeloma, diffuse large B-cell lymphoma, or non-Hodgkin's lymphoma. Disorders that may be treated include neurological disorders, such as Parkinson's disease, Alzheimers disease, multiple sclerosis, Amyotrophic lateral sclerosis, Huntington's disease, mild cognitive impairment, amyloidosis, AIDS-related dementia, encephalitis, stroke, head trauma, epilepsy, mood disorders, and dementia. As described herein, without being limited by mechanism, the ability of aryl hydrocarbon receptor modulators to treat such disorders may be due, in part, to an immune mechanism. Additional diseases treatable using aryl hydrocarbon receptor antagonists include myelodysplastic syndrome.

As used herein, the terms “treat”, “treating” or “treatment” refer to a method of alleviating or abating a disease and/or its attendant symptoms. As used herein, the terms “preventing” or “prevent” describes reducing or eliminating the onset of the symptoms or complications of the disease, condition, or disorder. As used herein, the terms “disease(s)”, “disorder(s)”, and “condition(s)” are used interchangeably, unless the context clearly dictates otherwise.

“Treating” may refer to therapeutic treatment, in which the object is to prevent or slow down (lessen) an undesired physiological change or disorder or to promote a beneficial phenotype in the patient being treated. Beneficial or desired clinical results include, but are not limited to, the observation of a reduction in quantity of a disease-causing cell population, such as a population of cancer cells or autoimmune cells.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein may retain or improve upon the biological activity of the original material.

As used herein, the term “alkyl” refers to a straight- or branched-chain alkyl group having, for example, from 1 to 20 carbon atoms in the chain, or, in certain embodiments, from 1 to 6 carbon atoms in the chain. Examples of alkyl groups include, but are not limited to, methyl, ethyl, n-propyl, isopropyl, butyl, isobutyl, sec-butyl, tert-butyl, pentyl, isopentyl, tert-pentyl, neopentyl, isopentyl, tert-pentyl, hexyl, isohexyl, and the like.

As used herein, the term “alkylene” refers to a straight- or branched-chain divalent alkyl group. The divalent positions may be on the same or different atoms within the alkyl chain. Examples of alkylene include methylene, ethylene, propylene, isopropylene, and the like.

As used herein, the term “heteroalkyl” refers to a straight or branched-chain alkyl group having, for example, from 1 to 20 carbon atoms in the chain, and further containing one or more heteroatoms (e.g., oxygen, nitrogen, or sulfur, among others) in the chain.

As used herein, the term “heteroalkylene” refers to a straight- or branched-chain divalent heteroalkyl group. The divalent positions may be on the same or different atoms within the heteroalkyl chain. The divalent positions may be one or more heteroatoms.

As used herein, the term “alkenyl” refers to a straight- or branched-chain alkenyl group having, for example, from 2 to 20 carbon atoms in the chain. It denotes a monovalent group derived from a hydrocarbon moiety containing, for example, from two to six carbon atoms having at least one carbon-carbon double bond. The double bond may or may not be the point of attachment to another group.

Examples of alkenyl groups include, but are not limited to, vinyl, propenyl, isopropenyl, butenyl, tert-butylenyl, 1-methyl-2-buten-1-yl, hexenyl, and the like.

As used herein, the term “alkenylene” refers to a straight- or branched-chain divalent alkenyl group. The divalent positions may be on the same or different atoms within the alkenyl chain. Examples of alkenylene include ethenylene, propenylene, isopropenylene, butenylene, and the like.

As used herein, the term “heteroalkenyl” refers to a straight- or branched-chain alkenyl group having, for example, from 2 to 20 carbon atoms in the chain, and further containing one or more heteroatoms (e.g., oxygen, nitrogen, or sulfur, among others) in the chain.

As used herein, the term “heteroalkenylene” refers to a straight- or branched-chain divalent heteroalkenyl group. The divalent positions may be on the same or different atoms within the heteroalkenyl chain. The divalent positions may be one or more heteroatoms.

As used herein, the term “alkynyl” refers to a straight- or branched-chain alkynyl group having, for example, from 2 to 20 carbon atoms in the chain and at least one carbon-carbon triple bond. Examples of alkynyl groups include, but are not limited to, propargyl, butynyl, pentynyl, hexynyl, and the like.

As used herein, the term “alkynylene” refers to a straight- or branched-chain divalent alkynyl group. The divalent positions may be on the same or different atoms within the alkynyl chain.

As used herein, the term “heteroalkynyl” refers to a straight- or branched-chain alkynyl group having, for example, from 2 to 20 carbon atoms in the chain, and further containing one or more heteroatoms (e.g., oxygen, nitrogen, or sulfur, among others) in the chain.

As used herein, the term “heteroalkynylene” refers to a straight- or branched-chain divalent heteroalkynyl group. The divalent positions may be on the same or different atoms within the heteroalkynyl chain. The divalent positions may be one or more heteroatoms.

As used herein, the term “cycloalkyl” refers to a monocyclic, or fused, bridged, or spiro polycyclic ring structure that is saturated and has, for example, from 3 to 12 carbon ring atoms. Examples of cycloalkyl groups include cyclopropyl, cyclobutyl, cyclopentyl, cyclohexyl, cycloheptyl, cyclooctyl, bicyclo[3.1.0]hexane, and the like. Also contemplated is a monovalent group derived from a monocyclic or polycyclic carbocyclic ring compound having at least one carbon-carbon double bond by the removal of at least one or two hydrogen atoms. Examples of such groups include, but are not limited to, cyclopropenyl, cyclobutenyl, cyclopentenyl,

As used herein, the term “cycloalkylene” refers to a divalent cycloalkyl group. The divalent positions may be on the same or different atoms within the ring structure. Examples of cycloalkylene include cyclopropylene, cyclobutylene, cyclopentylene, cyclohexylene, and the like.

As used herein, the term “heterocyloalkyl” or “heterocyclyl” refers to a monocyclic, or fused, bridged, or spiro polycyclic ring structure that is saturated and has, for example, from 3 to 12 ring atoms per ring structure selected from carbon atoms and heteroatoms selected from, e.g., nitrogen, oxygen, and sulfur, among others. The ring structure may contain, for example, one or more oxo groups on carbon, nitrogen, or sulfur ring members. Exemplary heterocycloalkyl groups include, but are not limited to, [1,3]dioxolane, pyrrolidinyl, pyrazolinyl, pyrazolidinyl, imidazolinyl, imidazolidinyl, piperazinyl, piperidinyl, oxazolidinyl, isooxazolidinyl, morpholinyl, thiazololidinyl, isothiazolidinyl, and tetrahydrofuryl.

As used herein, the term “heterocycloalkylene” refers to a divalent heterocyclolalkyl group. The divalent positions may be on the same or different atoms within the ring structure.

As used herein, the term “aryl” refers to a monocyclic or multicyclic aromatic ring system containing, for example, from 6 to 19 carbon atoms. Aryl groups include, but are not limited to, phenyl, fluorenyl, naphthyl, tetrahydronaphthyl, indanyl, indenyl, and the like. The divalent positions may be one or more heteroatoms.

As used herein, the term “arylene” refers to a divalent aryl group. The divalent positions may be on the same or different atoms.

As used herein, the term “heteroaryl” refers to a monocyclic heteroaromatic, or a bicyclic or a tricyclic fused-ring heteroaromatic group. In certain embodiments, the heteroaryl group contains five to ten ring atoms of which one ring atom is selected from S, O, and N; zero, one, or two ring atoms are additional heteroatoms independently selected from S. O, and N; and the remaining ring atoms are carbon. Heteroaryl groups include, but are not limited to, pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadia-zolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxazolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetriazolyl, 5,6,7,8-tetrahydroquinolyl, 5,6,7,8-tetrahydroisoquinolyl, purinyl, pteridinyl, carbazolyl, xanthenyl, benzoquinolyl, and the like.

As used herein, the term “heteroarylene” refers to a divalent heteroaryl group. The divalent positions may be on the same or different atoms. The divalent positions may be one or more heteroatoms.

Unless otherwise constrained by the definition of the individual substituent, the foregoing chemical moieties, such as “alkyl”, “alkylene”, “heteroalkyl”, ‘heteroalkylene’, “alkenyl”, “alkenylene”, “heteroalkenyl”, “heteroalkenylene”, “alkynyl”, “alkynylene”, “heteroalkynyl”, “heteroalkynylene”, “cycloalkyl”, “cycloalkylene”, “heterocyclolalkyl”, heterocycloalkylene”, “aryl,” “arylene”, “heteroaryl”, and “heteroarylene” groups can optionally be substituted. As used herein, the term “optionally substituted” refers to a compound or moiety containing one or more (for example, 1, 2, 3, 4, 5, 6, 7, 8, 9, 10, or more) substituents, as permitted by the valence of the compound or moiety or a site thereof, such as a substituent selected from the group consisting of alkyl, alkenyl, alkynyl, cycloalkyl, heterocycloalkyl, alkyl aryl, alkyl heteroaryl, alkyl cycloalkyl, alkyl heterocycloalkyl, amino, ammonium, acyl, acyloxy, acylamino, aminocarbonyl, alkoxycarbonyl, ureido, carbamate, aryl, heteroaryl, sulfinyl, sulfonyl, alkoxy, sulfanyl, halogen, carboxy, tihalomethyl, cyano, hydroxy, mercapto, nitro, and the like. The substitution may include situations in which neighboring substituents have undergone ring closure, such as ring closure of vicinal functional substituents, to form, for instance, lactams, lactones, cyclic anhydrides, acetals, hemiacetals, thioacetals, aminals, and hemiaminals, formed by ring closure, for example, to fumish a protecting group.

As used herein, the term “optionally substituted” refers to a chemical moiety that may have one or more chemical substituents, as valency permits, such as C1-4 alkyl, C1-4 alkenyl, C1-4 alkynyl, C₂₋₁₀ cycloalkyl, C₂₋₁₀ heterocyclolalkyl, C₂₋₁₀ aryl, C₂₋₁₀ alkylaryl, C₂₋₁₀ heteroaryl, C₂₋₁₀ alkylheteroaryl, amino, ammonium, acyl, acyloxy, acylamino, aminocarbonyl, alkoxycarbonyl, ureido, carbamate, sulfinyl, sulfonyl, alkoxy, sulfanyl, halogen, carboxy, trihalomethyl, cyano, hydroxy, mercapto, nitro, and the like. An optionally substituted chemical moiety may contain, e.g., neighboring substituents that have undergone ring closure, such as ring closure of vicinal functional substituents, thus forming, e.g., lactams, lactones, cyclic anhydrides, acetals, thioacetals, or aminals formed by ring closure, for instance, in order to generate protecting group.

In accordance with the application, any of the aryls, substituted aryls, heteroaryls and substituted heteroaryls described herein, can be any aromatic group.

The terms “hal,” “halo,” and “halogen,” as used herein, refer to an atom selected from fluorine, chlorine, bromine and iodine.

As described herein, compounds of the application and moieties present in the compounds may optionally be substituted with one or more substituents, such as are illustrated generally above, or as exemplified by particular classes, subclasses, and species of the application. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. The terms “optionally substituted”, “optionally substituted alkyl,” “optionally substituted alkenyl,” “optionally substituted alkynyl”, “optionally substituted cycloalkyl,” “optionally substituted cycloalkenyl,” “optionally substituted aryl”, “optionally substituted heteroaryl,” “optionally substituted aralkyl”, “optionally substituted heteroaralkyl,” “optionally substituted heterocycloalkyl,” and any other optionally substituted group as used herein, refer to groups that are substituted or unsubstituted by independent replacement of one, two, or three or more of the hydrogen atoms thereon with substituents including, but not limited to:

—F, —Cl, —Br, —I, —OH, protected hydroxy, —NO₂, —CN, —NH₂, protected amino, —NH—C₁-C₁₂-alkyl, —NH—C₂-C₁₂-alkenyl, —NH—C₂-C₁₂-alkenyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₁-C₁₂-alkenyl, —O—C₂-C₁₂-alkenyl, —O—C₃-C₁₂-cycloalkyl, —O-aryl, —O-heteroaryl, —O-heterocycloalkyl, —C(O)—C₁-C₁₂alkyl, —C(O)—C₁-C₁₂-alkenyl, —C(O)—C₂-C₁₂-alkenyl, —C(O)—C₃-C₁₂-cycloalkyl, —C(O)-aryl, —C(O)-heteroaryl, —C(O)— heterocycloalkyl, —CONH₂, —CONH—C₁-C₁₂-alkyl, —CONH—C₂-C₁₂-alkenyl, —CONH—C₁-C₁₂-alkenyl, —CONH—C₃-C₁₂-cycloalkyl, —CONH-aryl, —CONH-heteroaryl, —CONH-heterocycloalkyl, —OCO₂—C₁-C₁₂-alkyl, —OCO₂—C₂-C₁₂-alkenyl, —OCO₁—C₁-C₁₂-alkenyl, —OCO₂—C₃-C₁₂-cycloalkyl, —OCO₂-aryl, —OCO₂-heteroaryl, —OCO₂-heterocycloalkyl, —OCONH₂, —OCONH—C₁-C₁₂-alkyl, —OCONH—C₁-C₁₂-alkenyl, —OCONH—C₂-C₁₂-alkenyl, —OCONH—C₃-C₁₂-cycloalkyl, —OCONH-aryl, —OCONH-heteroaryl, —OCONH-heterocycloalkyl, —NHC(O)—C₁-C₁₂-alkyl, —NHC(O)—C₂-C₁₂-alkenyl, —NHC(O)—C₂-C₁₂-alkenyl, —NHC(O)—C₁-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₁₂-alkenyl, —NHCO₂—C₂-C₁₂-alkenyl, —NHCO₂—C₃-C₁₂-cycloalkyl, —NHCO₂-aryl, —NHCO₂-heteroaryl, —NHCO₂-heterocycloalkyl, NHC(O)NH₂, —NHC(O)NH—C₁-C₁₂-alkyl, —NHC(O)NH—C₂-C₁₂-alkenyl, —NHC(O)NH—C₂-C₁₂-alkenyl, —NHC(O)NH—C₃-C₁₂-cycloalkyl, —NHC(O)NH-aryl, —NHC(O)NH-heteroaryl, NHC(O)NH— heterocycloalkyl, —NHC(S)NH₂, —NHC(S)NH—C₁-C₁₂alkyl, —NHC(S)NH—C₂-C₁₂-alkenyl, —NHC(S)NH—C₁-C₁₂-alkenyl, —NHC(S)NH—C₃-C₁₂-cycloalkyl, —NHC(S)NH-aryl, —NHC(S)NH-heteroaryl, —NHC(S)NH— heterocycloalkyl, —NHC(NH)NH₂, —NHC(NH)NH—C₁-C₁₂-alkyl, —NHC(NH)NH—C₂-C₁₂-alkenyl, —NHC(NH)NH—C₂-C₁₂-alkenyl, —NHC(NH)NH—C₃-C₁₂-cycloalkyl, —NHC(NH)NH-aryl, —NHC(NH)NH-heteroaryl, —NHC(NH)NHheterocycloalkyl, —NHC(NH)—C₁-C₁₂-alkyl, —NHC(NH)—C₁-C₁₂-alkenyl, —NHC(NH)—C₂-C₁₂-alkenyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₁-C₁₂-alkenyl, —C(NH)NH—C₂-C₁₂-alkenyl, C(NH)NH—C₃-C₁₂-cycloalkyl, —C(NH)NH-aryl, —C(NH)NH-heteroaryl, —C(NH)NHheterocycloalkyl, —S(O)—C₁-C₁₂alkyl, —S(O)—C₁-C₁₂-alkenyl, —S(O)—C₂-C₁₂-alkenyl, —S(O)—C₁-C₁₂-cycloalkyl, —S(O)-aryl, —S(O)-heteroaryl, —S(O)-heterocycloalkyl-SO₂NH₂, —SO₂NH—C₁-C₁₂-alkyl, —SO₂NH—C₂-C₁₂-alkenyl, —SO₂NH—C₂-C₁₂-alkenyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₁₂-alkenyl, —NHSO₂—C₂-C₁₂-alkenyl, —NHSO₂—C₃-C₁₂-cycloalkyl, —NHSOraryl, —NHSOrheteroaryl, —NHSOrheterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₁₂-alkenyl, —S—C₂-C₁₂-alkenyl, —S—C₃-C₁₂-cycloalkyl, —S-aryl, —S-heteroaryl, —S— heterocycloalkyl, or methylthiomethyl.

### Aryl Hydrocarbon Receptor Antagonists

In some embodiments, the compound is an aryl hydrocarbon receptor antagonist.

In some embodiments, the aryl hydrocarbon receptor antagonist is a substituted imidazopyridine or imidazopyrazine.

In some embodiments, the aryl hydrocarbon receptor antagonist is a compound represented by formula (I) or formula (II):

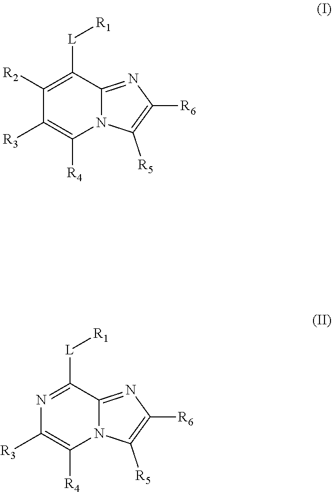

or a pharmaceutically acceptable salt, hydrate, or solvate thereof,

wherein L is a linker selected from the group consisting of —NR₇ₐ(CR₈ₐR_{8b})ₙ—, —O(CR₈ₐR_{8b})ₙ—, —C(O)(CR₈ₐR_{8b})ₙ—, —C(S)(CR₈ₐR_{8b})ₙ—, —S(O)₀₋₂(CR₈ₐR_{8b})ₙ—, —(CR₈ₐR_{8b})ₙ—, —NR₇ₐC(O)(CR₈ₐR_{8b})ₙ—, —NR₇ₐC(S)(CR₈ₐR_{8b})ₙ—, —OC(O)(CR₈ₐR_{8b})ₙ—, —OC(S)(CR₈ₐR_{8b})ₙ—, —C(O)NR₇ₐ(CR₈ₐR_{8b})ₙ—, —C(S)NR₇ₐ(CR₈ₐR_{8b})ₙ—, —C(O)O(CR₈ₐR_{8b})ₙ—, —C(S)O(CR₈ₐR_{8b})ₙ—, —S(O)₂NR₇ₐ(CR₈ₐR_{8b})ₙ—, —NR₇ₐS(O)₂(CR₈ₐR_{8b})ₙ—, —NR₇ₐC(O)NR_{7b}(CR₈ₐR_{8b})ₙ—, and —NR₇ₐC(O)O(CR₈ₐR_{8b})ₙ—, wherein R₇ₐ, R_{7b}, R₈ₐ, and R_{8b} are each independently selected from the group consisting of hydrogen and optionally substituted C1-4 alkyl, and each n is independently an integer from 2 to 6;

R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉ₐ, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)CR₉ₐR_{9b}R_{9c}, —OC(S)CR₉ₐR_{9b}R_{9c}, optionally substituted aryl, optionally substituted heteroaryl, optionally substituted cycloalkyl, and optionally substituted heterocycloalkyl, wherein R₉ₐ, R_{9b}, and R_{9c} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

R₂ is selected from the group consisting of hydrogen and optionally substituted C1-4 alkyl;

R₃ is selected from the group consisting of optionally substituted aryl, optionally substituted heteroaryl, optionally substituted cycloalkyl, and optionally substituted heterocycloalkyl;

R₄ is selected from the group consisting of hydrogen and optionally substituted C1-4 alkyl;

R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and

R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

or a salt thereof.

In some embodiments, R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉, —S(O)₀₋₂R₉, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)CR₉ₐR_{9b}R_{9c}, —OC(S)CR₉ₐR_{9b}R_{9c}, phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted, for example, with from 1 to 3 substituents independently selected from the group consisting of cyano, hydroxy, C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl. For instance, R₁ may be selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉ₐ, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)CR₉ₐR_{9b}R_{9c}, and —OC(S)CR₉ₐR_{9b}R_{9c}. R₁ may be selected from the group consisting of phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}.

In some embodiments, R₂ is hydrogen.

In some embodiments, R₃ is selected from the group consisting of phenyl, thiophenyl, furanyl, 1H-benzoimidazolyl, quinolinyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl, wherein the phenyl, thiophenyl, furanyl, 1H-benzoimidazolyl, quinolinyl, isoquinolinyl, imidazopyridinyl, benzothiophenyl, pyrimidinyl, pyridinyl, 1H-imidazolyl, pyrazinyl, pyridazinyl, 1H-pyrrolyl, or thiazolyl is optionally substituted with from 1 to 3 substituents independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}, and wherein R₁₁ₐ and R_{11b} are each independently selected from the group consisting of hydrogen and C₁₋₄ alkyl.

In some embodiments, R₄ is hydrogen.

In some embodiments, R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4alkyl.

In some embodiments, R₅ is selected from the group consisting of isopropyl, methyl, ethyl, prop-1-en-2-yl, isobutyl, cyclohexyl, sec-butyl, (S)-sec-butyl, (R)-sec-butyl, 1-hydroxypropan-2-yl, (S)-1-hydroxypropan-2-yl, (R)-1-hydroxypropan-2-yl, and nonan-2-yl.

In some embodiments, R₅ is (S)-1-hydroxypropan-2-yl.

In some embodiments, R₅ is (R)-1-hydroxypropan-2-yl.

In some embodiments, R₅ is (S)-sec-butyl.

In some embodiments, R₅ is (R)-sec-butyl.

In some embodiments, R₅ is selected from the group consisting of (i), (ii), (iii), (iv), and (v)

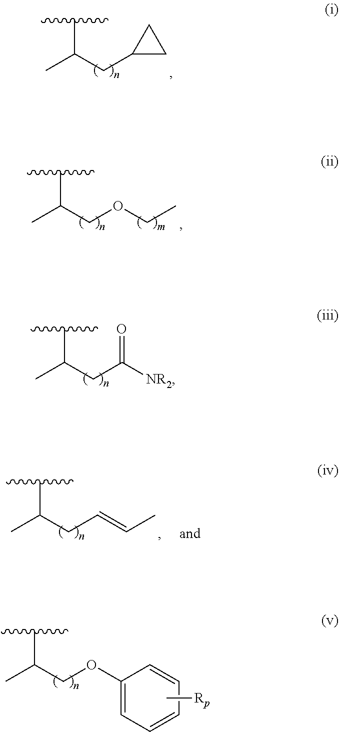

wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C1-4 alkyl.

In some embodiments, R₅ is selected from the group consisting of:

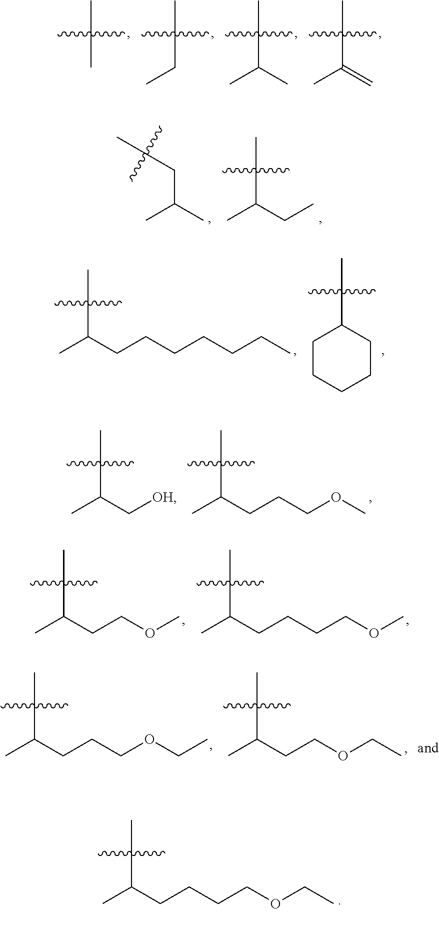

in some embodiments, R₅ is (ii);

In some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-8-ethoxyhexan-2-yl, and (R)-6-ethoxyhexan-2-yl.

In some embodiments, R₅ is (S)-4-methoxybutan-2-yl.

In some embodiments, R₅ is (R)-4-methoxybutan-2-yl.

In some embodiments, R₅ is (S)-5-methoxypentan-2-yl.

In some embodiments, R₅ is (R)-5-methoxypentan-2-yl.

In some embodiments, R₅ is (S)-4-ethoxybutan-2-yl.

In some embodiments, R₅ is (R)-4-ethoxybutan-2-yl.

Particular aryl hydrocarbon receptor antagonists described herein include:

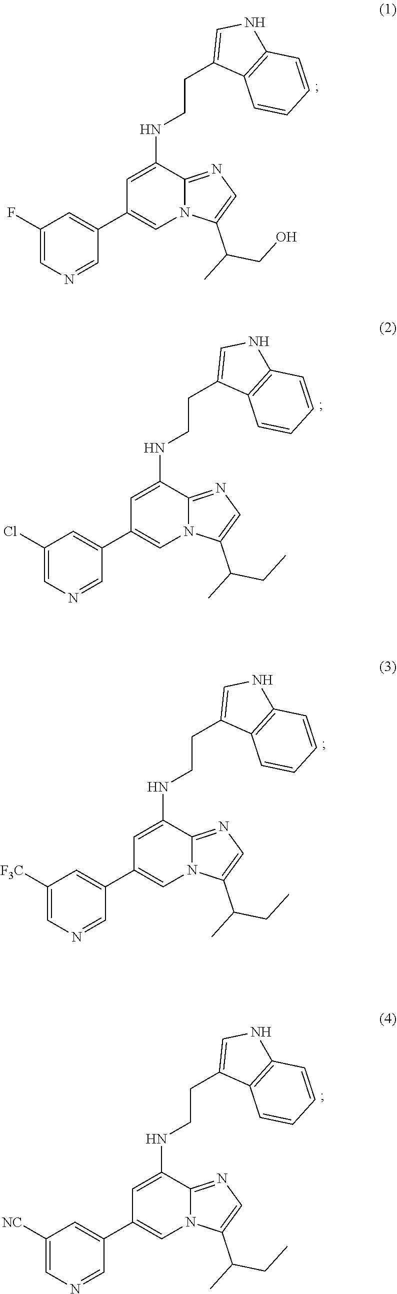 [SMILES: CC(CO)c1cnc2c(NCCc3c[nH]c4ccccc34)cc(-c3cncc(F)c3)cn12 ; CCC(C)c1cnc2c(NCCc3c[nH]c4ccccc34)cc(-c3cncc(Cl)c3)cn12 ; CCC(C)c1cnc2c(NCCc3c[nH]c4ccccc34)cc(-c3cncc(C(F)(F)F)c3)cn12 ; CCC(C)c1cnc2c(NCCc3c[nH]c4ccccc34)cc(-c3cncc(C#N)c3)cn12]

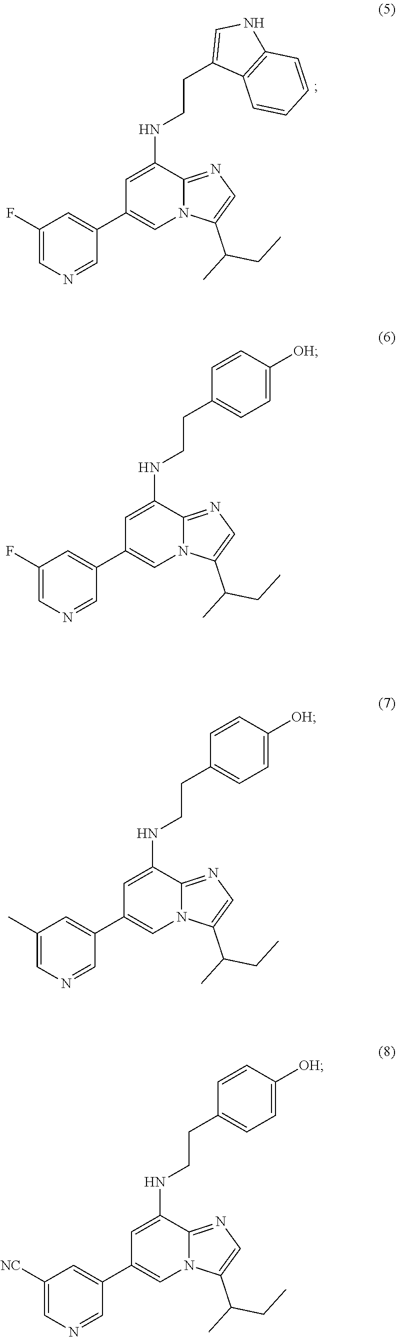 [SMILES: CCC(C)c1cnc2c(NCCc3c[nH]c4ccccc34)cc(-c3cncc(F)c3)cn12 ; CCC(C)c1cnc2c(NCCc3ccc(O)cc3)cc(-c3cncc(F)c3)cn12 ; CCC(C)c1cnc2c(NCCc3ccc(O)cc3)cc(-c3cncc(C)c3)cn12 ; CCC(C)c1cnc2c(NCCc3ccc(O)cc3)cc(-c3cncc(C#N)c3)cn12]

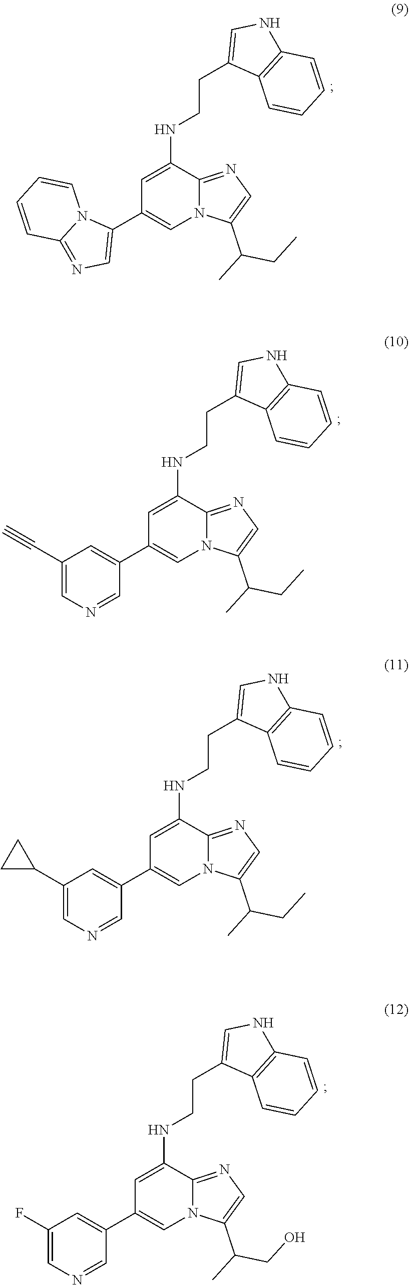 [SMILES: CCC(C)c1cnc2c(NCCc3c[nH]c4ccccc34)cc(-c3cnc4ccccn34)cn12 ; C#Cc1cncc(-c2cc(NCCc3c[nH]c4ccccc34)c3ncc(C(C)CC)n3c2)c1 ; CCC(C)c1cnc2c(NCCc3c[nH]c4ccccc34)cc(-c3cncc(C4CC4)c3)cn12 ; CC(CO)c1cnc2c(NCCc3c[nH]c4ccccc34)cc(-c3cncc(F)c3)cn12]

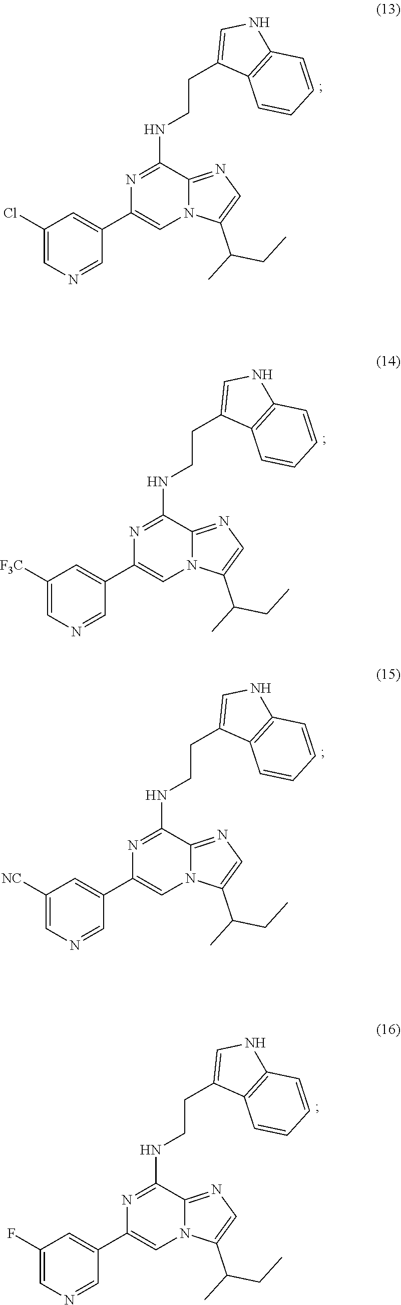 [SMILES: CCC(C)c1cnc2c(NCCc3c[nH]c4ccccc34)nc(-c3cncc(Cl)c3)cn12 ; CCC(C)c1cnc2c(NCCc3c[nH]c4ccccc34)nc(-c3cncc(C(F)(F)F)c3)cn12 ; CCC(C)c1cnc2c(NCCc3c[nH]c4ccccc34)nc(-c3cncc(C#N)c3)cn12 ; CCC(C)c1cnc2c(NCCc3c[nH]c4ccccc34)nc(-c3cncc(F)c3)cn12]

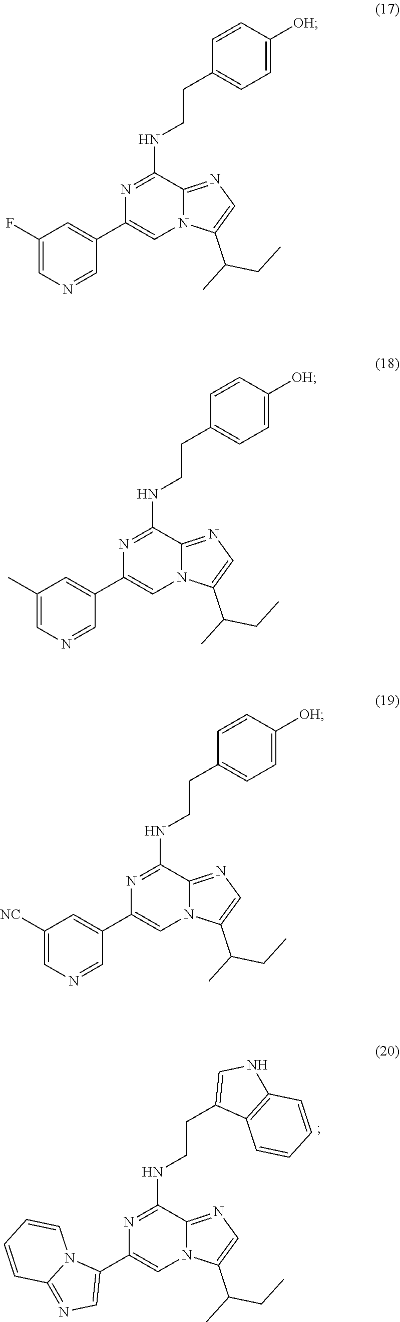 [SMILES: CCC(C)c1cnc2c(NCCc3ccc(O)cc3)nc(-c3cncc(F)c3)cn12 ; CCC(C)c1cnc2c(NCCc3ccc(O)cc3)nc(-c3cncc(C)c3)cn12 ; CCC(C)c1cnc2c(NCCc3ccc(O)cc3)nc(-c3cncc(C#N)c3)cn12 ; CCC(C)c1cnc2c(NCCc3c[nH]c4ccccc34)nc(-c3cnc4ccccn34)cn12]

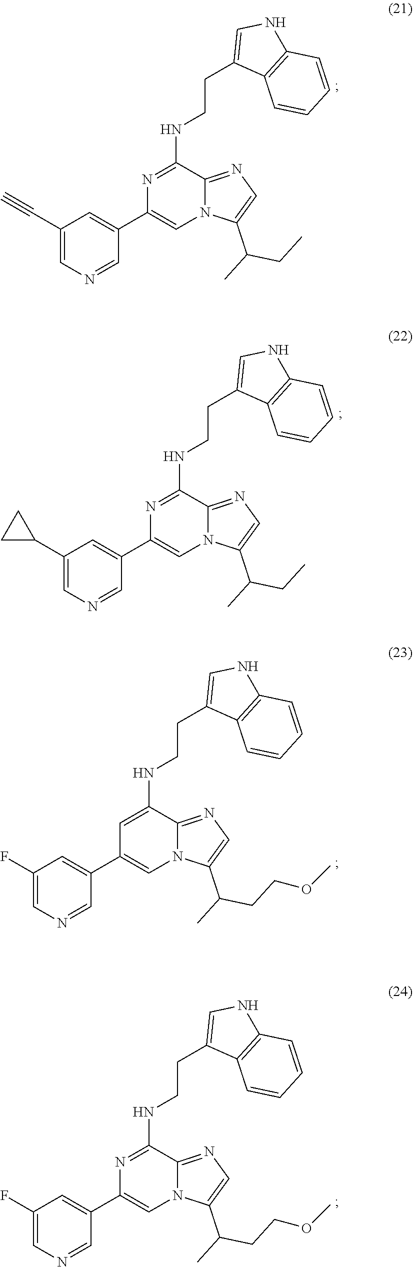 [SMILES: C#Cc1cncc(-c2cn3c(C(C)CC)cnc3c(NCCc3c[nH]c4ccccc34)n2)c1 ; CCC(C)c1cnc2c(NCCc3c[nH]c4ccccc34)nc(-c3cncc(C4CC4)c3)cn12 ; COCCC(C)c1cnc2c(NCCc3c[nH]c4ccccc34)cc(-c3cncc(F)c3)cn12 ; COCCC(C)c1cnc2c(NCCc3c[nH]c4ccccc34)nc(-c3cncc(F)c3)cn12]

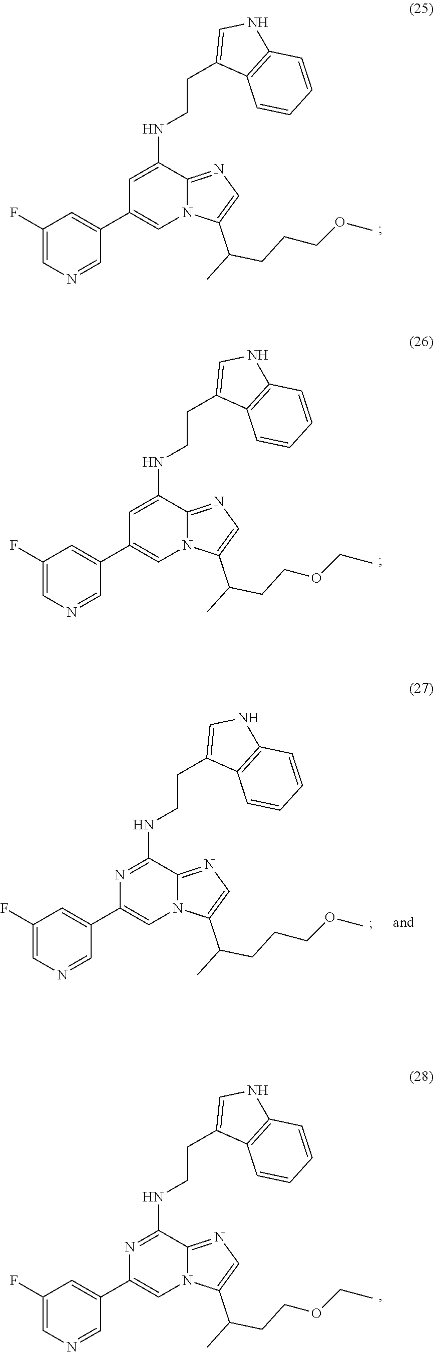 [SMILES: COCCCC(C)c1cnc2c(NCCc3c[nH]c4ccccc34)cc(-c3cncc(F)c3)cn12 ; CCOCCC(C)c1cnc2c(NCCc3c[nH]c4ccccc34)cc(-c3cncc(F)c3)cn12 ; COCCCC(C)c1cnc2c(NCCc3c[nH]c4ccccc34)nc(-c3cncc(F)c3)cn12 ; CCOCCC(C)c1cnc2c(NCCc3c[nH]c4ccccc34)nc(-c3cncc(F)c3)cn12]

as well as salts thereof.

In some embodiments, the aryl hydrocarbon receptor antagonist is a compound represented by formula (I)

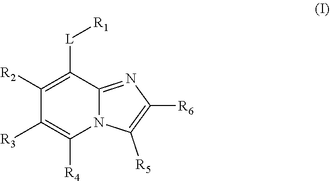

or a pharmaceutically acceptable salt, hydrate, or solvate thereof,

wherein L is a linker selected from the group consisting of —NR₇ₐ(CR₈ₐR_{8b})ₙ—, —O(CR₈ₐR_{8b})ₙ—, —C(O)(CR₈ₐR_{8b})ₙ—, —C(S)(CR₈ₐR_{8b})ₙ—, —S(O)₀₋₂(CR₈ₐR_{8b})ₙ—, —(CR₈ₐR_{8b})ₙ—, —NR₇ₐC(O)(CR₈ₐR_{8b})ₙ—, —NR₇ₐC(S)(CR₈ₐR_{8b})ₙ—, —OC(O)(CR₈ₐR_{8b})ₙ—, —OC(S)(CR₈ₐR_{8b})ₙ—, —C(O)NR₇ₐ(CR₈ₐR_{8b})ₙ—, —C(S)NR₇ₐ(CR₈ₐR_{8b})ₙ—, —C(O)O(CR₈ₐR_{8b})ₙ—, —C(S)O(CR₈ₐR_{8b})ₙ—, —S(O)₂NR₇ₐ(CR₈ₐR_{8b})ₙ—, —NR₇ₐS(O)₂(CR₈ₐR_{8b})ₙ—, —NR₇ₐC(O)NR_{7b}(CR₈ₐR_{8b})ₙ—, —NR₇ₐ(CR₈ₐR_{8b})ₙNR₇ₐ—, —NR₇ₐ(CR₈ₐR_{8b})ₙO—, —NR₇ₐ(CR₈ₐR_{8b})ₙS—, —O(CR₈ₐR_{8b})ₙNR₇ₐ—, —O(CR₈ₐR_{8b})ₙO—, —O(CR₈ₐR_{8b})ₙS—, —S(CR₈ₐR_{8b})ₙNR₇ₐ—, —S(CR₈ₐR_{8b})ₙO—, —S(CR₈ₐR_{8b})ₙS—, and —NR₇ₐC(O)O(CR₈ₐR_{8b})ₙ—, wherein R₇ₐ, R_{7b}, R₈ₐ, and R₈ₐ are each independently selected from the group consisting of hydrogen and optionally substituted C1-4 alkyl, and each n is independently an integer from 2 to 6;

R₁ is selected from the group consisting of —S(O)₂NR₉ₐR_{9b}, —NR₉ₐC(O)R_{9b}, —NR₉ₐC(S)R_{9b}, —NR₉ₐC(O)NR₉ₐR_{9c}, —C(O)R₉ₐ, —C(S)R₉ₐ, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)CR₉ₐR_{9b}R_{9c}, —OC(S)CR₉ₐR_{9b}R_{9c}, optionally substituted aryl, optionally substituted heteroaryl, optionally substituted cycloalkyl, and optionally substituted heterocycloalkyl, wherein R₉ₐ, R_{9b}, and R_{9c} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

R₂ is selected from the group consisting of hydrogen and optionally substituted C1-4 alkyl;

R₃ is selected from the group consisting of optionally substituted aryl, optionally substituted heteroaryl, optionally substituted cycloalkyl, and optionally substituted heterocycloalkyl;

R₄ is selected from the group consisting of hydrogen and optionally substituted C1-4 alkyl;

R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and

R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

or a salt thereof.

As used herein to describe linkers (represented by “L” in formulas (I), (II), and the like), the notation “-(Linker)-” (wherein “linker” is represented using chemical symbols such as NR₇ₐ(CR₈ₐR_{8b})ₙ, O(CR₈ₐR_{8b})ₙ, C(O)(CR₈ₐR_{8b})ₙ, C(S)(CR₈ₐR_{8b})ₙ, S(O)₀₋₂(CR₈ₐR_{8b})ₙ, (CR₈ₐR_{8b})ₙ, NR₇ₐC(O)(CR₈ₐR_{8b})ₙ, NR₇ₐC(S)(CR₈ₐR_{8b})ₙ, OC(O)(CR₈ₐR_{8b})ₙ, OC(S)(CR₈ₐR_{8b})ₙ, C(O)NR₇ₐ(CR₈ₐR_{8b})ₙ, C(S)NR₇ₐ(CR₈ₐR_{8b})ₙ, C(O)O(CR₈ₐR_{8b})ₙ, C(S)O(CR₈ₐR_{8b})ₙ, S(O)₂NR₇ₐ(CR₈ₐR_{8b})ₙ, NR₇ₐS(O)₂(CR₈ₐR_{8b})ₙ, and NR₇ₐC(O)NR_{7b}(CR₈ₐR_{8b})ₙ) designates that the left hyphen represents a covalent bond to the indicated position on the imidazopyridine or imidazopyrazine ring system, while the right hyphen represents a covalent bond to R₁.

In some embodiments, R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉ₐ, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂R %, —NR₉ₐC(O)OR_{9b}, —OC(O)CR₉ₐR_{9b}R_{9c}, —OC(S)CR₉ₐR_{9b}R_{9c}, phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted, for example, with from 1 to 3 substituents independently selected from the group consisting of cyano, hydroxy, C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

In some embodiments, R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉ₐ, —S(O)₀₋₂R₉ₐ, —C(O)OR₉ₐ, —C(S)OR_{9b}, —C(O)NR₉ₐR_{9b}, —C(S)NR₉ₐR_{9b}, —NR₉ₐS(O)₂R_{9b}, —NR₉ₐC(O)OR_{9b}, —OC(O)CR₉ₐR_{9b}R_{9c}, and —OC(S)CR₉ₐR_{9b}R_{9c}.

In some embodiments, R₁ is selected from the group consisting of phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted, for example, with from 1 to 3 substituents independently selected from the group consisting of cyano, hydroxy, C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}.

In some embodiments, R₁ is selected from the group consisting of phenyl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-tiazol-5-yl, 2-oxoimidazolidin-1-yl, 1H-pyrazol-3-yl, 1H-pyrazol-4-yl, and 2-oxo-2,3-dihydro-1H-benzo[d]imidazol-5-yl, wherein the phenyl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, or 2-oxo-2,3-dihydro-1H-benzo[d]imidazol-5-yl is optionally substituted, for example, with from 1 to 3 substituents independently selected from the group consisting of cyano, hydroxy. C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}.

In some embodiments, R₁ is selected from the group consisting of phenyl, phenol-4-yl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-tiazol-5-yl, 2-oxoimidazolidin-1-yl, 1H-pyrazol-3-yl, 1H-pyrazol-4-yl, and 2-oxo-2,3-dihydro-1H-benzo[d]imidazol-5-yl.

In some embodiments, R₁ is selected from the group consisting of:

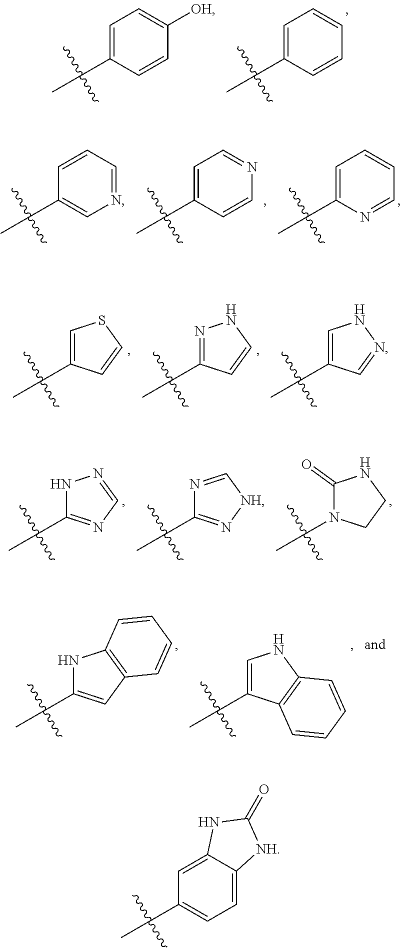

In some embodiments, R₁ is selected from the group consisting of

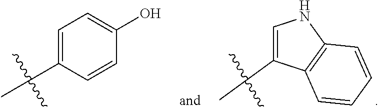 [SMILES: *c1ccc(O)cc1 ; *c1c[nH]c2ccccc12]

In some embodiments, R₁ is selected from the group consisting of phenol-4-yl and 1H-indol-3-yl.

In some embodiments, L is selected from the group consisting of —NR₇ₐ(CR₈ₐR_{8b})) and —O(CR₈ₐR_{8b})ₙ—.

In some embodiments, L is selected from the group consisting of —NH(CH₂)₂— and —O(CH₂)₂—.

In some embodiments, R₂ is hydrogen.

In some embodiments, R₃ is selected from the group consisting of optionally substituted aryl and optionally substituted heteroaryl.

In some embodiments, R₃ is selected from the group consisting of phenyl, thiophenyl, furanyl, 1H-benzoimidazolyl, quinolinyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl, wherein the phenyl, thiophenyl, furanyl, 1H-benzoimidazolyl, quinolinyl, isoquinolinyl, imidazopyridinyl, benzothiophenyl, pyrimidinyl, pyridinyl, 1H-imidazolyl, pyrazinyl, pyridazinyl, 1H-pyrrolyl, or thiazolyl is optionally substituted, for example, with from 1 to 3 substituents independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}, and wherein R₁₁ₐ and R_{11b} are each independently selected from the group consisting of hydrogen and C₁₋₄ alkyl.

In some embodiments, R₃ is selected from the group consisting of thiophen-2-yl, thiophen-3-yl, furan-3-yl, 1H-benzo[d]imidazol-1-yl, isoquinolin-4-yl, 1H-imidazo[4,5-b]pyridin-1-yl, imidazo[1,2-a]pyridin-3-yl, benzo[b]thiophen-3-yl, pyrimidin-5-yl, pyridin-2-yl, pyridin-3-yl, pyridin-4-yl, 1H-imidazol-1-yl, pyrazin-2-yl, pyridazin-4-yl, 1H-pyrrol-2-yl and thiazol-5-yl, wherein the thiophen-2-yl, thiophen-3-yl, furan-3-yl, 1H-benzo[d]imidazol-1-yl, isoquinolin-4-yl, 1H-imidazo[4,5-b]pyridin-1-yl, benzo[b]thiophen-3-yl, pyrimidin-5-yl, pyridin-2-yl, pyridin-3-yl, pyridin-4-yl, 1H-imidazol-1-yl, pyrazin-2-yl, pyridazin-4-yl, 1H-pyrrol-2-yl, or thiazol-5-yl is optionally substituted, for example, with from 1 to 3 substituents independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}.

In some embodiments, R₃ is selected from the group consisting of thiophen-3-yl, benzo[b]thiophen-3-yl, pyridin-3-yl, pyrimidin-5-yl, 1H-imidazol-1-yl, 1H-benzo[d]imidazol-1-yl, isoquinolin-4-yl, 1H-imidazo[4,5-b]pyridin-1-yl, and imidazo[1,2-a]pyridin-3-yl, wherein the thiophen-3-yl, benzo[b]thiophen-3-yl, pyridin-3-yl, pyrimidin-5-yl, 1H-imidazol-1-yl, 1H-benzo[d]imidazol-1-yl, isoquinolin-4-yl, 1H-imidazo[4,5-b]pyridin-1-yl, or imidazo[1,2-a]pyridin-3-yl is optionally substituted, for example, with from 1 to 3 substituents independently selected from the group consisting of cyano, hydroxy, C1-4 alkyl, C₁-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}.

In some embodiments, R₃ is selected from the group consisting of optionally substituted:

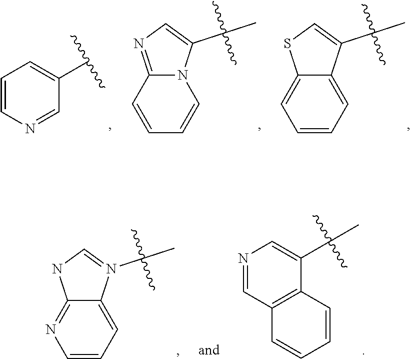

In some embodiments, R₃ is pyridin-3-yl, wherein the pyridin-3-yl is optionally substituted at C5, for example, with a substituent selected from the group consisting of C₁-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

In some embodiments, the pyridin-3-yl is substituted at C5 with a substituent selected from the group consisting of ethoxycarbonyl, methoxy, cyano, methyl, methylsulfonyl, fluoro, chloro, trifluoromethyl, ethynyl, and cyclopropyl.

In some embodiments, R₃ is selected from the group consisting of:

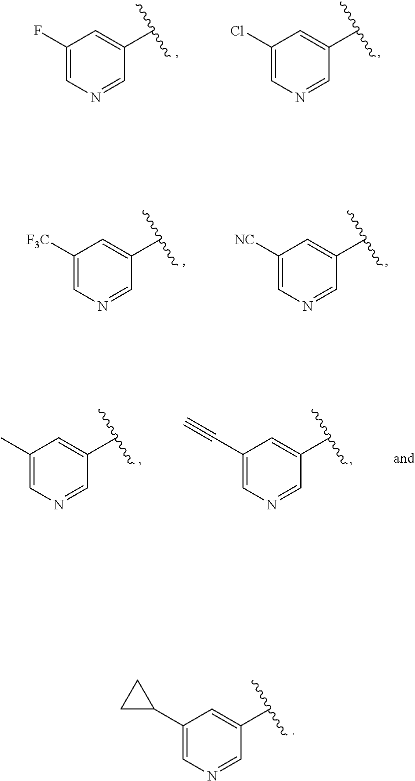

In some embodiments, R₃ is imidazo[1,2-a]pyridin-3-yl, wherein the imidazo[1,2-a]pyridin-3-yl is optionally substituted, for example, with a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

In some embodiments, R₃ is benzo[b]thiophen-3-yl, wherein the benzo[b]thiophen-3-yl is optionally substituted, for example, with a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

In some embodiments, R₃ is 1H-imidazo[4,5-b]pyridin-1-yl, wherein the 1H-imidazo[4,5-b]pyridin-1-yl is optionally substituted, for example, with a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

In some embodiments, R₃ is isoquinolin-4-yl, wherein the isoquinolin-4-yl is optionally substituted, for example, with a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

In some embodiments, R₄ is hydrogen.

In some embodiments, R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted, for example, with from 1 to 3 substituents independently selected from the group consisting of hydroxy, C1-4 alkyl, and halo-substituted-C1-4alkyl.

In some embodiments, R₅ is selected from the group consisting of isopropyl, methyl, ethyl, prop-1-en-2-yl, isobutyl, cyclohexyl, sec-butyl, (S)-sec-butyl, (R)-sec-butyl, 1-hydroxypropan-2-yl, (S)-1-hydroxypropan-2-yl, (R)-1-hydroxypropan-2-yl, and nonan-2-yl.

In some embodiments, R₅ is (S)-1-hydroxypropan-2-yl.

In some embodiments, R₅ is (R)-1-hydroxypropan-2-yl

In some embodiments, R₅ is (S)-sec-butyl.

In some embodiments, R₅ is (R)-sec-butyl.

In some embodiments, R₅ is selected from the group consisting of (i), (ii), (iii), (iv), and (v)

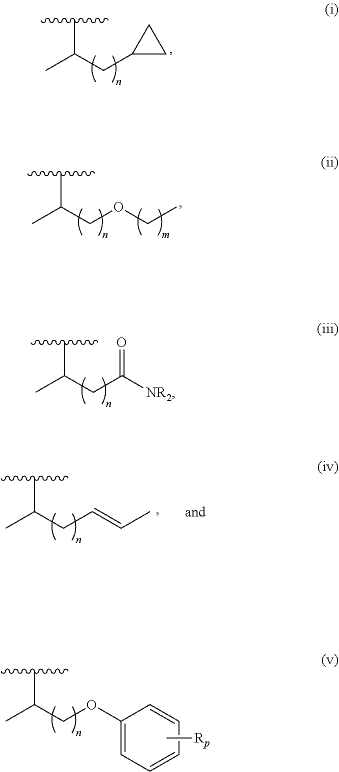

wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C1-4 alkyl.

In some embodiments, R₅ is selected from the group consisting of

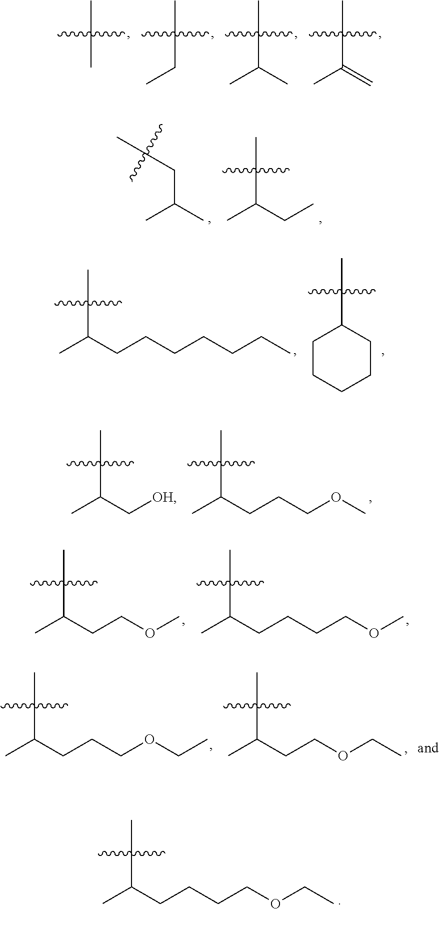

In some embodiments, R₅ is (ii).

In some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl.

In some embodiments, R₅ is (S)-4-methoxybutan-2-yl.

In some embodiments, R₅ is (R)-4-methoxybutan-2-yl.

In some embodiments, R₅ is (S)-5-methoxypentan-2-yl.

In some embodiments, R₅ is (R)-5-methoxypentan-2-yl.

In some embodiments, R₅ is (S)-4-ethoxybutan-2-yl.

In some embodiments, R₅ is (R)-4-ethoxybutan-2-yl.

In some embodiments, Re is hydrogen.

In some embodiments, the aryl hydrocarbon receptor antagonist is as compound represented by formula (I-a)

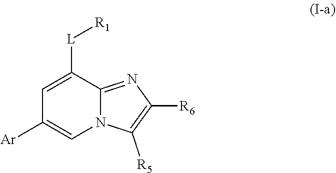

or a pharmaceutically acceptable salt, hydrate, or solvate thereof,

wherein L is a linker selected from the group consisting of —NR₇ₐ(CR₈ₐR_{8b})ₙ—, —O(CR₈ₐR_{8b})ₙ—, —C(O)(CR₈ₐR_{8b})ₙ—, —C(S)(CR₈ₐR_{8b})ₙ—, —S(O)₀₋₂(CR₈ₐR_{8b})ₙ—, —(CR₈ₐR_{8b})ₙ—, —NR₇ₐC(O)(CR₈ₐR_{8b})ₙ—, —NR₇ₐC(S)(CR₈ₐR_{8b})ₙ—, —OC(O)(CR₈ₐR_{8b})ₙ—, —OC(S)(CR₈ₐR_{8b})ₙ—, —C(O)NR₇ₐ(CR₈ₐR_{8b})ₙ—, —C(S)NR₇ₐ(CR₈ₐR_{8b})ₙ—, —C(O)O(CR₈ₐR_{8b})ₙ—, —C(S)O(CR₈ₐR_{8b})ₙ—, —S(O)₂NR₇ₐ(CR₈ₐR_{8b})ₙ—, —NR₇ₐS(O)₂(CR₈ₐR_{8b})ₙ—, —NR₇ₐC(O)NR_{7b}(CR₈ₐR_{8b})ₙ—, and —NR₇ₐC(O)O(CR₈ₐR_{8b})ₙ—, wherein R₇ₐ, R_{7b}, R_{7c}, and R₉ₐ are each independently selected from the group consisting of hydrogen and optionally substituted C1-4 alkyl, and each n is independently an integer from 2 to 6;

R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉ₐ, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)NR₉ₐR_{9b}, —OC(S)CR₉ₐR_{9b}R_{9c}, optionally substituted aryl, optionally substituted heteroaryl, optionally substituted cycloalkyl, and optionally substituted heterocycloalkyl, wherein R₉ₐ, R_{9b}, and R_{9c} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl (for example, R₁ may be selected from the group consisting of phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted, for example, with from 1 to 3 substituents independently selected from the group consisting of cyano, hydroxy, C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl);

Ar is selected from the group consisting of optionally substituted monocyclic aryl and heteroaryl, such as optionally substituted thiophenyl, furanyl, 1H-benzoimidazolyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl;

R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and

R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

or a salt thereof.

In some embodiments, Ar is pyridin-3-yl, wherein the pyridin-3-yl is optionally substituted at C5, for example, with a substituent selected from the group consisting of ethoxycarbonyl, methoxy, cyano, methyl, methylsulfonyl, fluoro, chloro, trifluoromethyl, ethynyl, and cyclopropyl.

In some embodiments, the aryl hydrocarbon receptor antagonist is a compound represented by formula (I-b)

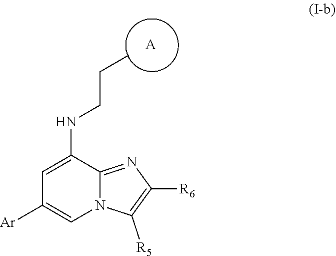

or a pharmaceutically acceptable salt, hydrate, or solvate thereof,

wherein A is an optionally substituted ring system selected from the group consisting of phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C1-4 alkyl, C₁₋₄ alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

Ar is selected from the group consisting of optionally substituted monocyclic aryl and heteroaryl, such as optionally substituted thiophenyl, furanyl, 1H-benzoimidazolyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl;

R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and

R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

or a salt thereof.

In some embodiments, A is selected from the group consisting of phenyl, phenol-4-yl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, and 2-oxo-2,3-dihydro-1H-benzo[d]imidazol-5-yl.

In some embodiments, A is selected from the group consisting of phenol-4-yl and 1H-indol-3-yl.

In some embodiments, the aryl hydrocarbon receptor antagonist is a compound represented by formula (I-c)

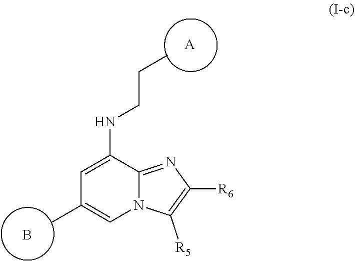

or a pharmaceutically acceptable salt, hydrate, or solvate thereof,

wherein A is an optionally substituted ring system selected from the group consisting of phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C1-4 alkyl, C₁₋₄ alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

B is an optionally substituted ring system selected from the group consisting of thiophenyl, furanyl, 1H-benzoimidazolyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl, wherein the thiophenyl, furanyl, 1H-benzoimidazolyl, isoquinolinyl, 1H-imidazopyridinyl, benzothiophenyl, pyrimidinyl, pyridinyl, 1H-imidazolyl, pyrazinyl, pyridazinyl, 1H-pyrrolyl, or thiazolyl is optionally substituted with from 1 to 3 substituents independently selected from the group consisting of cyano, hydroxy. C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄alkyl;

R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and

R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

or a salt thereof.

In some embodiments, B is pyridin-3-yl, wherein the pyridin-3-yl is optionally substituted at C5, for example, with a substituent selected from the group consisting of ethoxycarbonyl, methoxy, cyano, methyl, methylsulfonyl, fluoro, chloro, trifluoromethyl, ethynyl, and cyclopropyl.

In some embodiments, the aryl hydrocarbon receptor antagonist is a compound represented by formula (I-d)

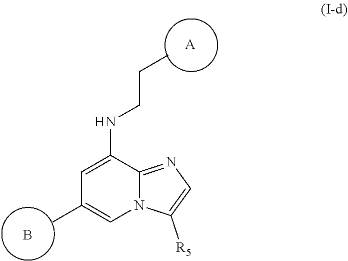

or a pharmaceutically acceptable salt, hydrate, or solvate thereof,

wherein A is an optionally substituted ring system selected from the group consisting of phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C1-4alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

B is an optionally substituted ring system selected from the group consisting of thiophenyl, furanyl, 1H-benzoimidazolyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl, wherein the thiophenyl, furanyl, 1H-benzoimidazolyl, isoquinolinyl, 1H-imidazopyridinyl, benzothiophenyl, pyrimidinyl, pyridinyl, 1H-imidazolyl, pyrazinyl, pyridazinyl, 1H-pyrrolyl, or thiazolyl is optionally substituted with from 1 to 3 substituents independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and

R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl;

or a salt thereof.

In some embodiments, the aryl hydrocarbon receptor antagonist is a compound represented by formula (I-e)

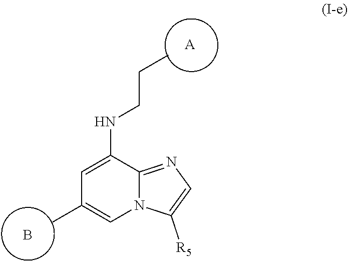

or a pharmaceutically acceptable salt, hydrate, or solvate thereof,

wherein A is an optionally substituted ring system selected from the group consisting of phenyl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, and 2-oxo-2,3-dihydro-1H-benzo[d]imidazol-5-yl, wherein the phenyl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, or 2-oxo-2,3-dihydro-1H-benzo[d]imidazol-5-yl is optionally substituted with from 1 to 3 substituents independently selected from the group consisting of cyano, hydroxy, C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

B is an optionally substituted ring system selected from the group consisting of thiophen-2-yl, thiophen-3-yl, furan-3-yl, 1H-benzo[d]imidazol-1-yl, isoquinolin-4-yl, 1H-imidazo[4,5-b]pyridin-1-yl, imidazo[1,2-a]pyridin-3-yl, benzo[b]thiophen-3-yl, pyrimidin-5-yl, pyridin-2-yl, pyridin-3-yl, pyridin-4-yl, 1H-imidazol-1-yl, pyrazin-2-yl, pyridazin-4-yl, 1H-pyrrol-2-yl and thiazol-5-yl, wherein the thiophen-2-yl, thiophen-3-yl, furan-3-yl, 1H-benzo[d]imidazol-1-yl, isoquinolin-4-yl, 1H-imidazo[4,5-b]pyridin-1-yl, benzo[b]thiophen-3-yl, pyrimidin-5-yl, pyridin-2-yl, pyridin-3-yl, pyridin-4-yl, 1H-imidazol-1-yl, pyrazin-2-yl, pyridazin-4-yl, 1H-pyrrol-2-yl, or thiazol-5-yl is optionally substituted with from 1 to 3 substituents independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and

R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4alkyl, or R₅ is selected from the group consisting of (i), (ii), (iii), (iv), and (v)

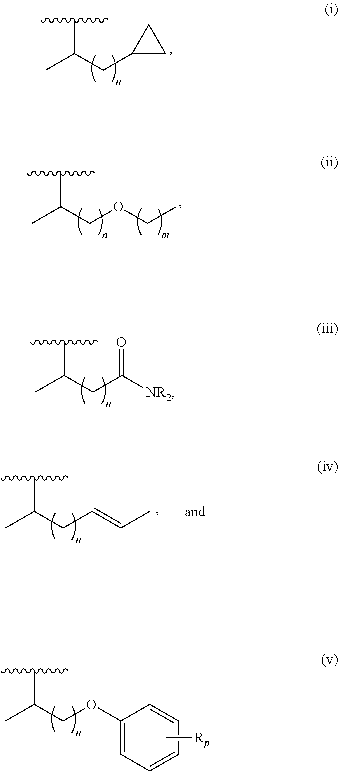

wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl;

In some embodiments, R₅ is selected from the group consisting of:

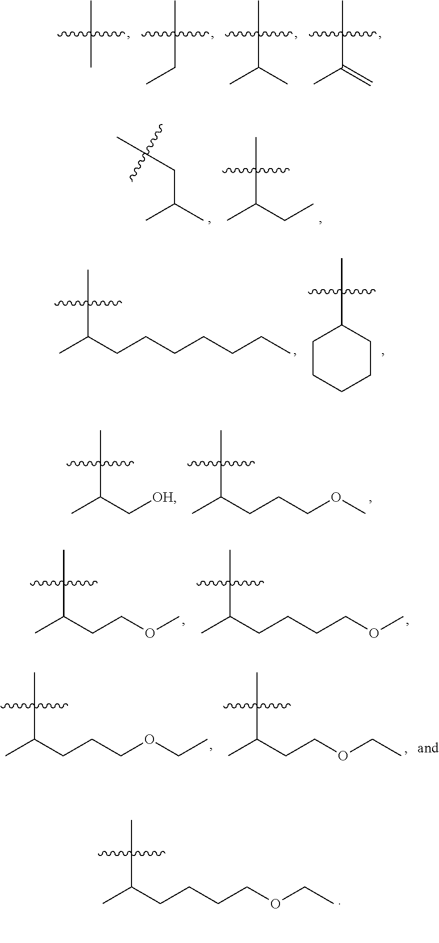

in some embodiments, R₅ is (ii);

in some embodiments, R₅ is selected from the group consisting of 4-methoxybutan-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl; or a salt thereof.

In some embodiments, the aryl hydrocarbon receptor antagonist is a compound represented by formula (I-f)

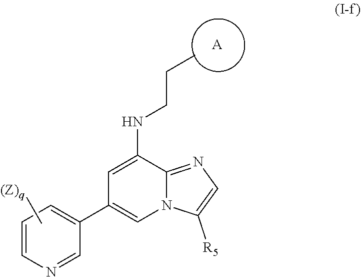

or a pharmaceutically acceptable salt, hydrate, or solvate thereof,

wherein A is an optionally substituted ring system selected from the group consisting of phenol-4-yl and 1H-indol-3-yl;

q is an integer from 0 to 4;

each Z is independently a substituent selected from the group consisting of C1-4 alkyl, halo, halo-substituted-C1-4 alkyl, C1-4 alkenyl, C1-4 alkynyl, C2-6 cycloalkyl. C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and

R₅ is selected from the group consisting of isopropyl, methyl, ethyl, prop-1-en-2-yl, isobutyl, cyclohexyl, sec-butyl, (S)-sec-butyl, (R)-sec-butyl, 1-hydroxypropan-2-yl, (S)-1-hydroxypropan-2-yl, (R)-1-hydroxypropan-2-yl, and nonan-2-yl, or R₅ is selected from the group consisting of (i), (ii), (iii), (iv), and (v)

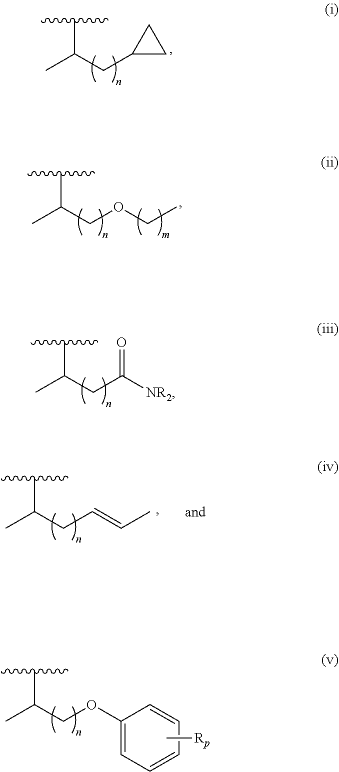

wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl;

In some embodiments, R₅ is selected from the group consisting of:

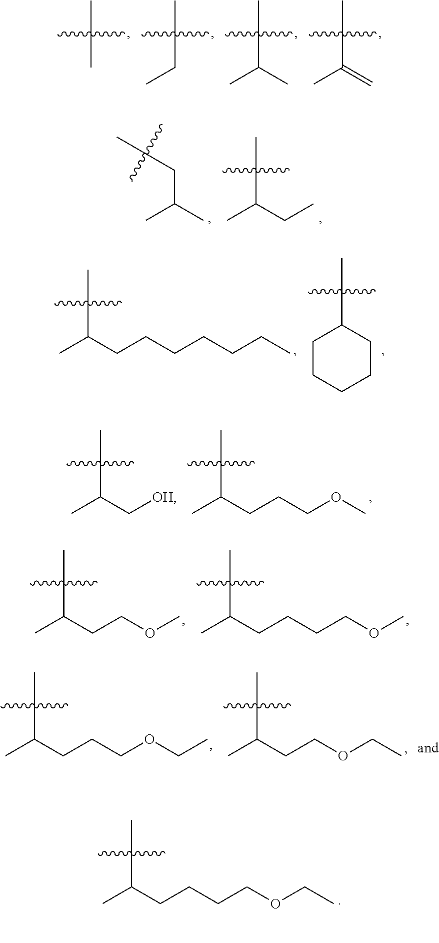

in some embodiments, R₅ is (ii);

in some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl;

or a salt thereof.

In some embodiments, each Z is independently a substituent selected from the group consisting of ethoxycarbonyl, methoxy, cyano, methyl, methylsulfonyl, fluoro, chloro, trifluoromethyl, ethynyl, and cyclopropyl.

In some embodiments, the aryl hydrocarbon receptor antagonist is a compound represented by formula (I-g)

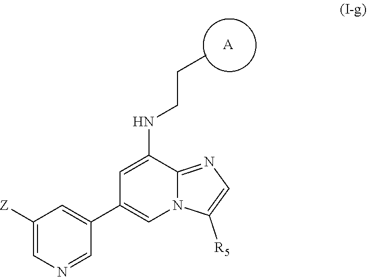

or a pharmaceutically acceptable salt, hydrate, or solvate thereof,

wherein A is an optionally substituted ring system selected from the group consisting of phenol-4-yl and 1H-indol-3-yl;

Z is a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and

R₅ is selected from the group consisting of isopropyl, methyl, ethyl, prop-1-en-2-yl, isobutyl, cyclohexyl, sec-butyl, (S)-sec-butyl, (R)-sec-butyl, 1-hydroxypropan-2-yl, (S)-1-hydroxypropan-2-yl, (R)-1-hydroxypropan-2-yl, and nonan-2-yl, or R₅ is selected from the group consisting of (i), (ii), (iii), (iv), and (v)

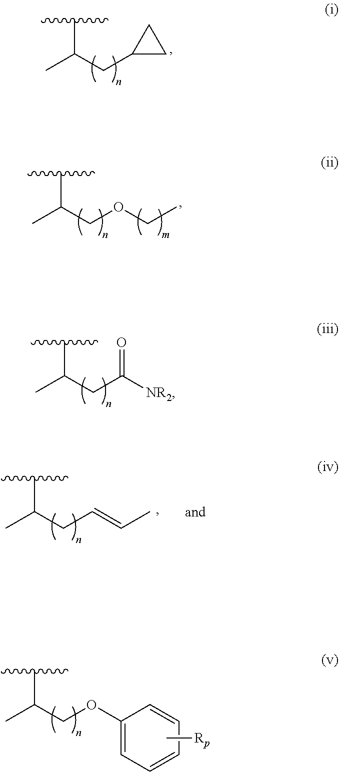

wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄alkyl;

In some embodiments, R₅ is selected from the group consisting of:

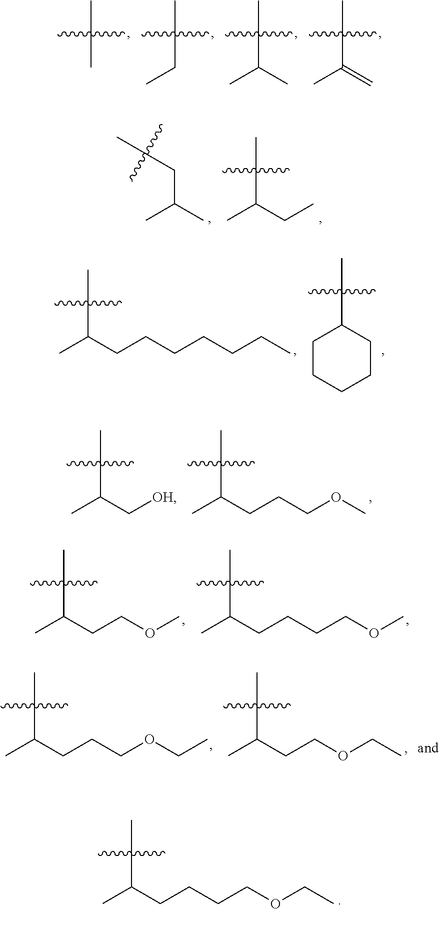

in some embodiments, R₅ is (ii);

in some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl;

or a salt thereof.

In some embodiments, the aryl hydrocarbon receptor antagonist is a compound represented by formula (I-h)

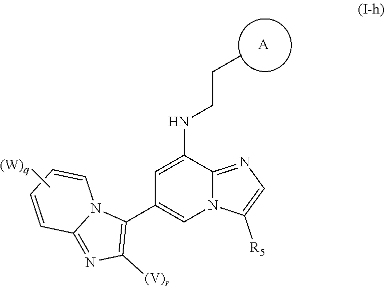

or a pharmaceutically acceptable salt, hydrate, or solvate thereof,

wherein A is an optionally substituted ring system selected from the group consisting of phenol-4-yl and 1H-indol-3-yl;

q is an integer from 0 to 4;

r is 0 or 1;

W and V are each independently a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄alkyl; and

R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4alkyl, or R₅ is selected from the group consisting of (i), (ii), (iii), (iv), and (v)

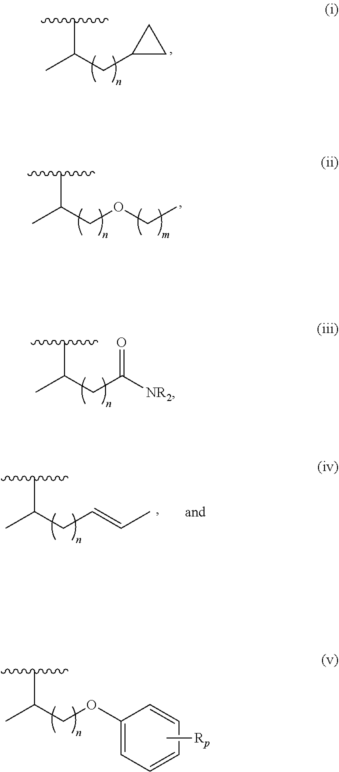

wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl;

In some embodiments, R₅ is selected from the group consisting of:

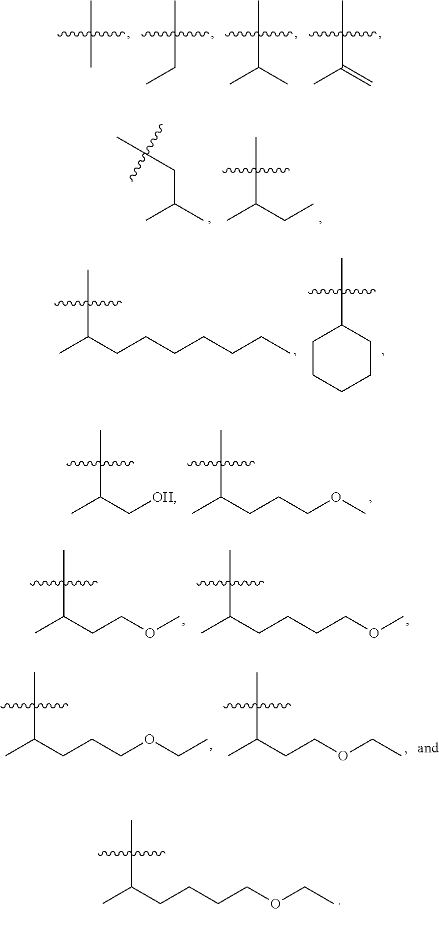

in some embodiments, R₅ is (ii);

in some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl;

or a salt thereof.

In some embodiments, the aryl hydrocarbon receptor antagonist is a compound represented by formula (I-i)

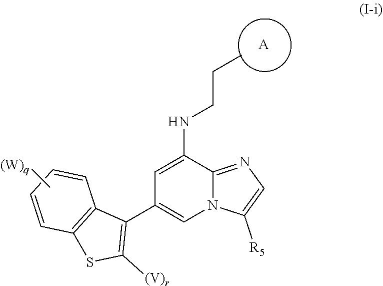

or a pharmaceutically acceptable salt, hydrate, or solvate thereof,

wherein A is an optionally substituted ring system selected from the group consisting of phenol-4-yl and 1H-indol-3-yl;

q is an integer from 0 to 4;

r is 0 or 1;

W and V are each independently a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{1b}1 are each independently selected from the group consisting of hydrogen and C₁₋₄alkyl; and

R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4alkyl, or R₅ is selected from the group consisting of (i), (i), (iii), (iv), and (v)

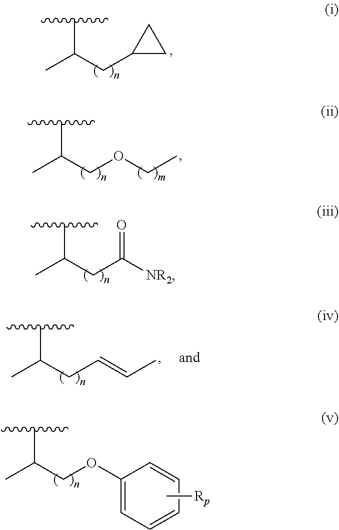

wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl;

In some embodiments, R₅ is selected from the group consisting of:

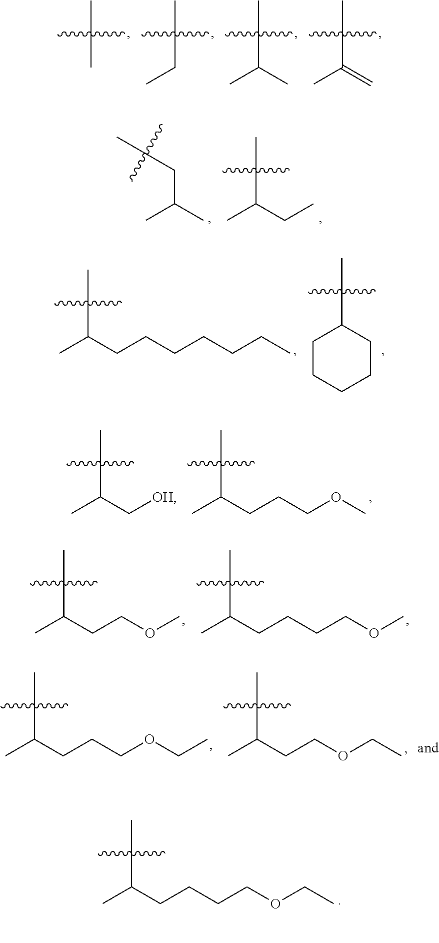

in some embodiments, R₅ is (ii);

in some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl;

or a salt thereof.

In some embodiments, the aryl hydrocarbon receptor antagonist is a compound represented by formula (I-j)

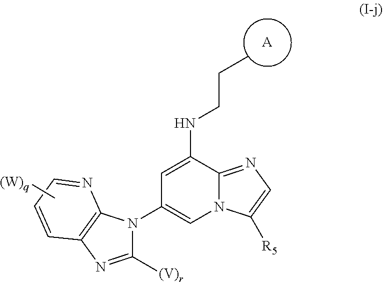

or a pharmaceutically acceptable salt, hydrate, or solvate thereof,

wherein A is an optionally substituted ring system selected from the group consisting of phenol-4-yl and 1H-indol-3-yl;

q is an integer from 0 to 4;

r is 0 or 1;

W and V are each independently a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{1b}1 are each independently selected from the group consisting of hydrogen and C₁₋₄alkyl; and

R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4alkyl, or R₅ is selected from the group consisting of (i), (ii), (iii), (iv), and (v)

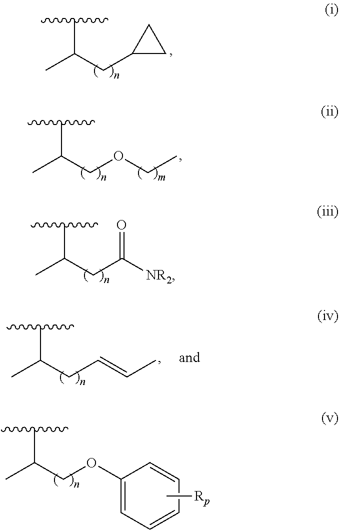

wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl;

In some embodiments, R₅ is selected from the group consisting of:

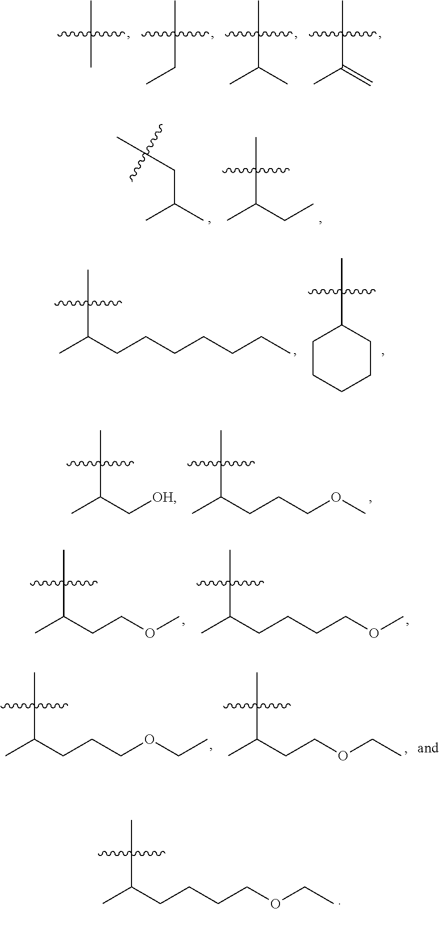

in some embodiments, R₅ is (ii);

in some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl;

or a salt thereof.

In some embodiments, the aryl hydrocarbon receptor antagonist is a compound represented by formula (I-k)

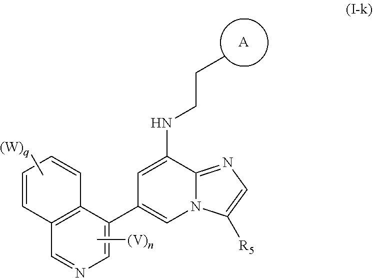

or a pharmaceutically acceptable salt, hydrate, or solvate thereof,

wherein A is an optionally substituted ring system selected from the group consisting of phenol-4-yl and 1H-indol-3-yl;

q is an integer from 0 to 4;

r is 0 or 1;

W and V are each independently a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{1b}1 are each independently selected from the group consisting of hydrogen and C₁₋₄alkyl; and

R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4alkyl, or R₅ is selected from the group consisting of (i), (ii), (iii), (iv), and (v)

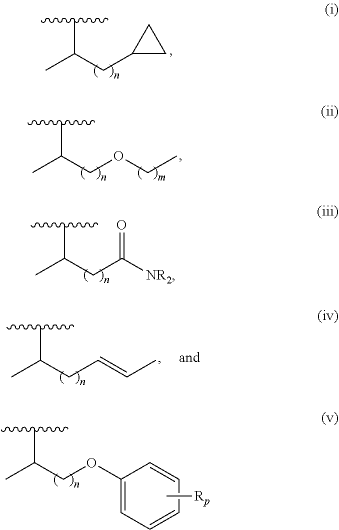

wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl;

In some embodiments, R₅ is selected from the group consisting of:

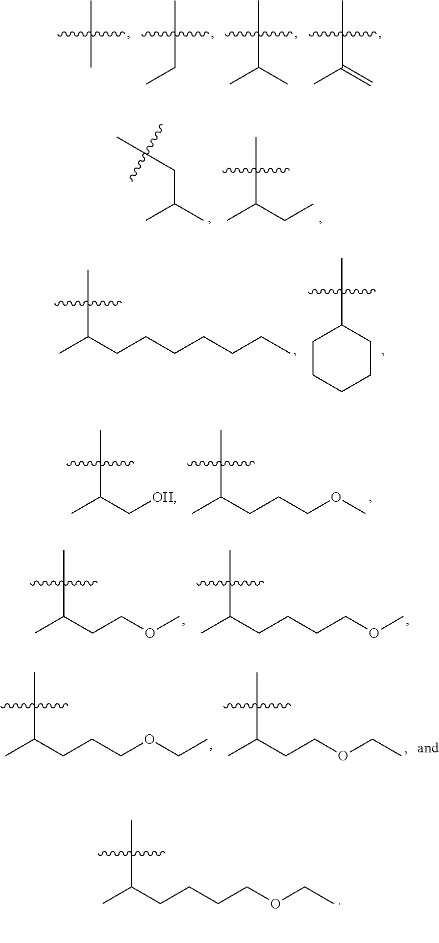

in some embodiments, R₅ is (ii);

in some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl;

or a salt thereof.

In some embodiments, the compound is compound (1)

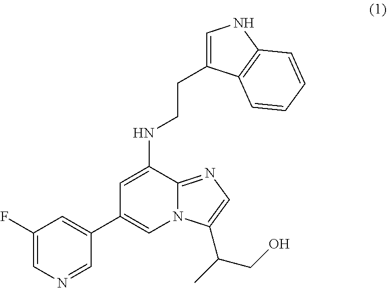 [SMILES: CC(CO)c1cnc2c(NCCc3c[nH]c4ccccc34)cc(-c3cncc(F)c3)cn12]

or a salt thereof.

In some embodiments, the compound is compound (2)

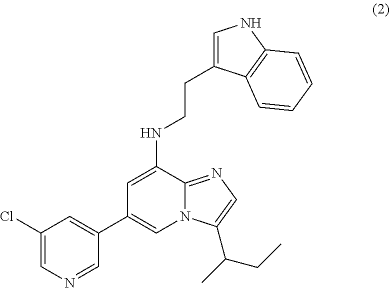 [SMILES: CCC(C)c1cnc2c(NCCc3c[nH]c4ccccc34)cc(-c3cncc(Cl)c3)cn12]

or a salt thereof.

In some embodiments, the compound is compound (3) 59

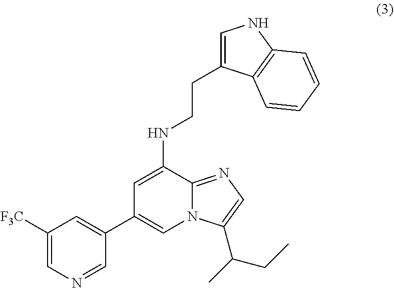 [SMILES: CCC(C)c1cnc2c(NCCc3c[nH]c4ccccc34)cc(-c3cncc(C(F)(F)F)c3)cn12]

or a salt thereof.

In some embodiments, the compound is compound (4)

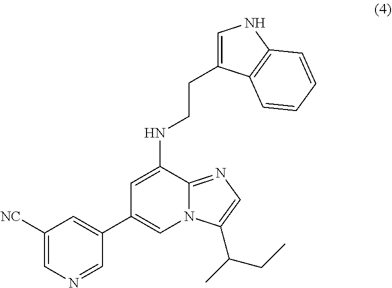 [SMILES: CCC(C)c1cnc2c(NCCc3c[nH]c4ccccc34)cc(-c3cncc(C#N)c3)cn12]

or a salt thereof.

In some embodiments, the compound is compound (5)

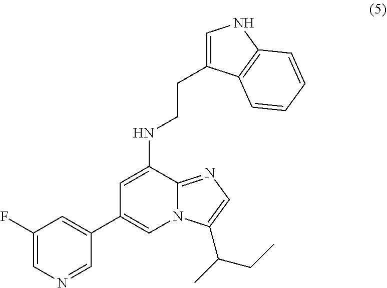 [SMILES: CCC(C)c1cnc2c(NCCc3c[nH]c4ccccc34)cc(-c3cncc(F)c3)cn12]

or a salt thereof.

In some embodiments, the compound is compound (6)

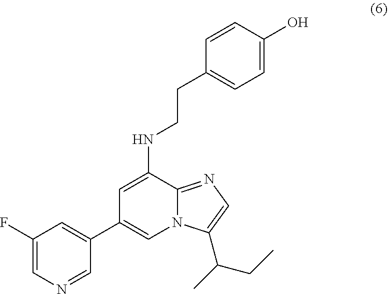 [SMILES: CCC(C)c1cnc2c(NCCc3ccc(O)cc3)cc(-c3cncc(F)c3)cn12]

or a salt thereof.

In some embodiments, the compound is compound (7)

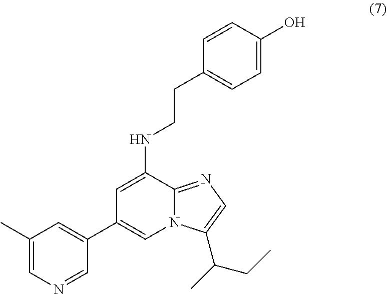 [SMILES: CCC(C)c1cnc2c(NCCc3ccc(O)cc3)cc(-c3cncc(C)c3)cn12]

or a salt thereof.

In some embodiments, the compound is compound (8)

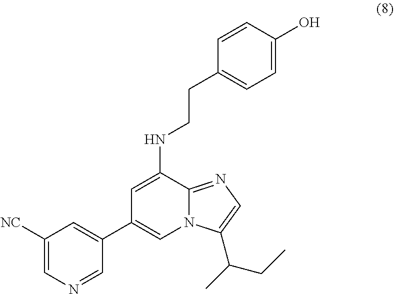 [SMILES: CCC(C)c1cnc2c(NCCc3ccc(O)cc3)cc(-c3cncc(C#N)c3)cn12]

or a salt thereof.

In some embodiments, the compound is compound (9)

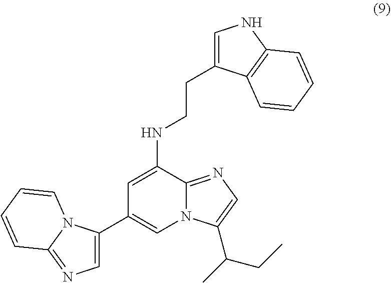 [SMILES: CCC(C)c1cnc2c(NCCc3c[nH]c4ccccc34)cc(-c3cnc4ccccn34)cn12]

or a salt thereof.

In some embodiments, the compound is compound (10)

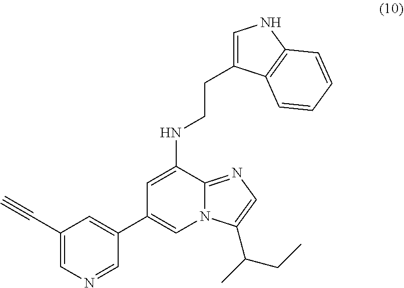 [SMILES: C#Cc1cncc(-c2cc(NCCc3c[nH]c4ccccc34)c3ncc(C(C)CC)n3c2)c1]

or a salt thereof.

In some embodiments, the compound is compound (11)

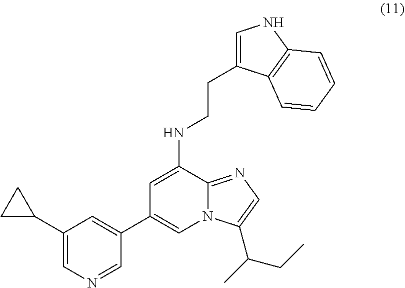 [SMILES: CCC(C)c1cnc2c(NCCc3c[nH]c4ccccc34)cc(-c3cncc(C4CC4)c3)cn12]

or a salt thereof.

In some embodiments, the compound is compound (23)

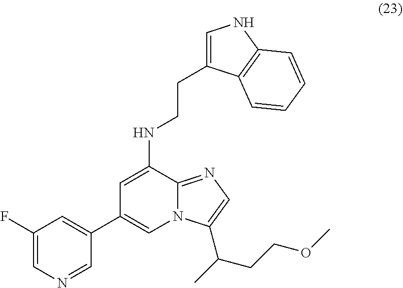 [SMILES: COCCC(C)c1cnc2c(NCCc3c[nH]c4ccccc34)cc(-c3cncc(F)c3)cn12]

or a salt thereof.

In some embodiments, the compound is compound (25)

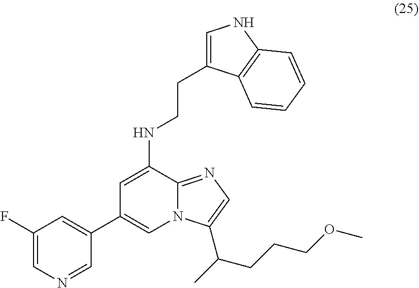 [SMILES: COCCCC(C)c1cnc2c(NCCc3c[nH]c4ccccc34)cc(-c3cncc(F)c3)cn12]

or a salt thereof.

In some embodiments, the compound is compound (26)

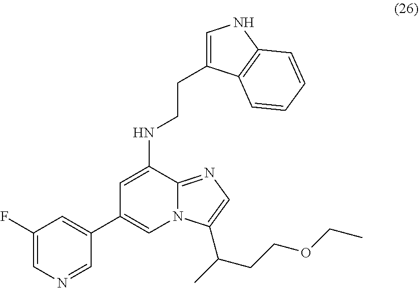 [SMILES: CCOCCC(C)c1cnc2c(NCCc3c[nH]c4ccccc34)cc(-c3cncc(F)c3)cn12]

or a salt thereof.

In some embodiments, the compound is a compound represented by formula (II)

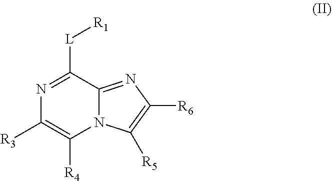

or a pharmaceutically acceptable salt, hydrate, or solvate thereof,

wherein L is a linker selected from the group consisting of —NR₇ₐ(CR₈ₐR_{8b})ₙ—, —O(CR₈ₐR_{8b})ₙ—, —C(O)(CR₈ₐR_{8b})ₙ—, —C(S)(CR₉ₐR_{9b})ₙ—, —S(O)₀₋₂(CR₈ₐR_{8b})ₙ—, —(CR₈ₐR_{8b})ₙ—, —NR₇ₐC(O)(CR₈ₐR_{8b})ₙ—, —NR₇ₐC(S)(CR₈ₐR_{8b})ₙ—, —OC(O)(CR₈ₐR_{8b})ₙ—, —OC(S)(CR₈ₐR_{8b})ₙ—, —C(O)NR₇ₐ(CR₈ₐR_{8b})ₙ—, —C(S)NR₇ₐ(CR₈ₐR_{8b})ₙ—, —C(O)O(CR₈ₐR_{8b})ₙ—, —C(S)O(CR₈ₐR_{8b})ₙ—, —S(O)₂NR₇ₐ(CR₈ₐR_{8b})ₙ—, —NR₇ₐS(O)₂(CR₈ₐR_{8b})ₙ—, —NR₇ₐC(O)NR_{7b}(CR₈ₐR_{8b})ₙ—, —NR₇ₐ(CR₈ₐR_{8b})ₙNR₇ₐ, —NR₇ₐ(CR₈ₐR_{8b})ₙO—, —NR₇ₐ(CR₈ₐR_{8b})ₙS—, —O(CR₈ₐR_{8b})ₙNR₇ₐ—, —O(CR₈ₐR_{8b})ₙO—, —O(CR₈ₐR_{8b})ₙS—, —S(CR₈ₐR_{8b})ₙNR₇ₐ—, —S(CR₈ₐR_{8b})ₙO—, —S(CR₈ₐR_{8b})ₙS—, and —NR₇ₐC(O)O(CR₈ₐR_{8b})ₙ—, wherein R₇ₐ, R_{7b}, R₈ₐ, and R_{8b} are each independently selected from the group consisting of hydrogen and optionally substituted C1-4 alkyl, and each n is independently an integer from 2 to 6;

R₁ is selected from the group consisting of —S(O)₂NR₉ₐR_{9b}, —NR₉ₐC(O)R_{9b}, —NR₉ₐC(S)R_{9b}, —NR₉ₐC(O)NR₉ₐR_{9b}, —C(O)R₉ₐ, —C(S)R₉ₐ, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)CR₉ₐR_{9b}, —OC(S)CR₉ₐR_{9b}R_{9c}, optionally substituted aryl, optionally substituted heteroaryl, optionally substituted cycloalkyl, and optionally substituted heterocycloalkyl, wherein R₉ₐ, R_{9b}, and R_{9c} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

R₃ is selected from the group consisting of optionally substituted aryl, optionally substituted heteroaryl, optionally substituted cycloalkyl, and optionally substituted heterocycloalkyl;

R₄ is selected from the group consisting of hydrogen and optionally substituted C1-4 alkyl;

R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and

R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

or a salt thereof.

In some embodiments, R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉ₐ, —S(O)₀₋₂R₉, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)CR₉ₐR_{9b}R_{9c}, —OC(S)CR₉ₐR_{9b}R_{9c}, phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted, for example, with from 1 to 3 substituents independently selected from the group consisting of cyano, hydroxy, C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}; wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

In some embodiments, R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉ₐ, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)CR₉ₐR_{9b}R_{9c}, and —OC(S)CR₉ₐR_{9b}R_{9c}.

In some embodiments, R₁ is selected from the group consisting of phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted, for example, with from 1 to 3 substituents independently selected from the group consisting of cyano, hydroxy, C1-4 alkyl, C₁₋₄ alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{9b}, and —NR₁₀ₐS(O)₂R_{10b}.

In some embodiments, R₁ is selected from the group consisting of phenyl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, and 2-oxo-2,3-dihydro-1H-benzo[d]imidazol-5-yl, wherein the phenyl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, or 2-oxo-2,3-dihydro-1H-benzo[d]imidazol-5-yl is optionally substituted, for example, with from 1 to 3 substituents independently selected from the group consisting of cyano, hydroxy. C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR₁₀ₐ and —NR₁₀ₐS(O)₂R_{10b}.

In some embodiments, R₁ is selected from the group consisting of phenyl, phenol-4-yl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, and 2-oxo-2,3-dihydro-1H-benzo[d]imidazol-5-yl.

In some embodiments, R₁ is selected from the group consisting of

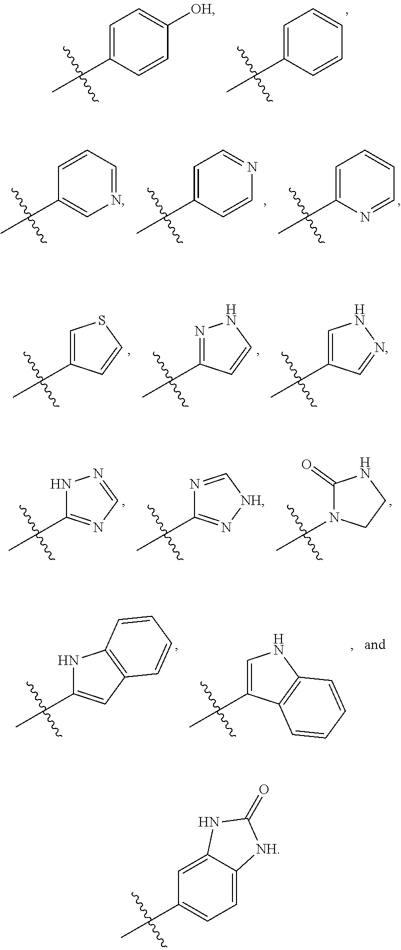

In some embodiments, R₁ is selected from the group consisting of

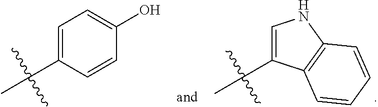 [SMILES: *c1ccc(O)cc1 ; *c1c[nH]c2ccccc12]

In some embodiments, R₁ is selected from the group consisting of phenol-4-yl and 1H-indol-3-yl.

In some embodiments, L is selected from the group consisting of —NR₇ₐ(CR₈ₐR_{8b}), and —O(CR₈ₐR_{8b})ₙ—.

In some embodiments, L is selected from the group consisting of —NH(CH₂)₂— and —O(CH₂)₂—.

In some embodiments, R₃ is selected from the group consisting of optionally substituted aryl and optionally substituted heteroaryl.

In some embodiments, R₃ is selected from the group consisting of phenyl, thiophenyl, furanyl, 1H-benzoimidazolyl, quinolinyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl, wherein the phenyl, thiophenyl, furanyl, 1H-benzoimidazolyl, quinolinyl, isoquinolinyl, imidazopyridinyl, benzothiophenyl, pyrimidinyl, pyridinyl, 1H-imidazolyl, pyrazinyl, pyridazinyl, 1H-pyrrolyl, or thiazolyl is optionally substituted, for example, with from 1 to 3 substituents independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}, and wherein R₁₁ₐ and R_{11b} are each independently selected from the group consisting of hydrogen and C₁₋₄ alkyl.

In some embodiments, R₃ is selected from the group consisting of thiophen-2-yl, thiophen-3-yl, furan-3-yl, 1H-benzo[d]imidazol-1-yl, isoquinolin-4-yl, 1H-imidazo[4,5-b]pyridin-1-yl, imidazo[1,2-a]pyridin-3-yl, benzo[b]thiophen-3-yl, pyrimidin-5-yl, pyridin-2-yl, pyridin-3-yl, pyridin-4-yl, 1H-imidazol-1-yl, pyrazin-2-yl, pyridazin-4-yl, 1H-pyrrol-2-yl and thiazol-5-yl, wherein the thiophen-2-yl, thiophen-3-yl, furan-3-yl, 1H-benzo[d]imidazol-1-yl, isoquinolin-4-yl, 1H-imidazo[4,5-b]pyridin-1-yl, benzo[b]thiophen-3-yl, pyrimidin-5-yl, pyridin-2-yl, pyridin-3-yl, pyridin-4-yl, 1H-imidazol-1-yl, pyrazin-2-yl, pyridazin-4-yl, 1H-pyrrol-2-yl, or thiazol-5-yl is optionally substituted, for example, with from 1 to 3 substituents independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}.

In some embodiments, R₃ is selected from the group consisting of thiophen-3-yl, benzo[b]thiophen-3-yl, pyridin-3-yl, pyrimidin-5-yl, 1H-imidazol-1-yl, 1H-benzo[d]imidazol-1-yl, isoquinolin-4-yl, 1H-imidazo[4,5-b]pyridin-1-yl, and imidazo[1,2-a]pyridin-3-yl, wherein the thiophen-3-yl, benzo[b]thiophen-3-yl, pyridin-3-yl, pyrimidin-5-yl, 1H-imidazol-1-yl, 1H-benzo[d]imidazol-1-yl, isoquinolin-4-yl, 1H-imidazo[4,5-b]pyridin-1-yl, or imidazo[1,2-a]pyridin-3-yl is optionally substituted, for example, with from 1 to 3 substituents independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}.

In some embodiments, R₃ is selected from the group consisting of optionally substituted:

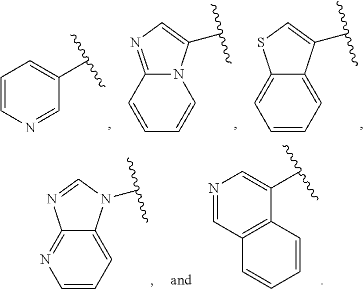

In some embodiments, R₃ is pyridin-3-yl, wherein the pyridin-3-yl is optionally substituted at C5, for example, with a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

In some embodiments, the pyridin-3-yl is substituted at C5 with a substituent selected from the group consisting of ethoxycarbonyl, methoxy, cyano, methyl, methylsulfonyl, fluoro, chloro, trifluoromethyl, ethynyl, and cyclopropyl.

In some embodiments, R₃ is selected from the group consisting of

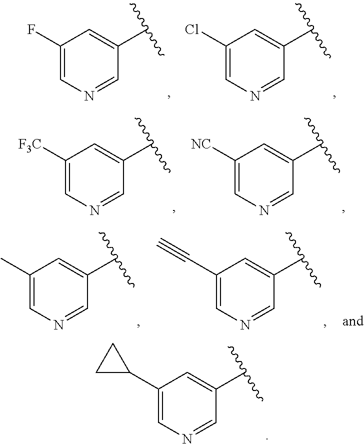

In some embodiments, R₃ is imidazo[1,2-a]pyridin-3-yl, wherein the imidazo[1,2-a]pyridin-3-yl is optionally substituted, for example, with a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

In some embodiments, R₃ is benzo[b]thiophen-3-yl, wherein the benzo[b]thiophen-3-yl is optionally substituted, for example, with a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

In some embodiments, R₃ is 1H-imidazo[4,5-b]pyridin-1-yl, wherein the 1H-imidazo[4,5-b]pyridin-1-yl is optionally substituted, for example, with a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

In some embodiments, R₃ is isoquinolin-4-yl, wherein the isoquinolin-4-yl is optionally substituted, for example, with a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

In some embodiments, R₄ is hydrogen.

In some embodiments, R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted, for example, with from 1 to 3 substituents independently selected from the group consisting of hydroxy, C1-4 alkyl, and halo-substituted-C1-4alkyl.

In some embodiments, R₅ is selected from the group consisting of isopropyl, methyl, ethyl, prop-1-en-2-yl, isobutyl, cyclohexyl, sec-butyl, (S)-sec-butyl, (R)-sec-butyl, 1-hydroxypropan-2-yl, (S)-1-hydroxypropan-2-yl, (R)-1-hydroxypropan-2-yl, and nonan-2-yl.

In some embodiments, R₅ is (S)-1-hydroxypropan-2-yl.

In some embodiments, R₅ is (R)-1-hydroxypropan-2-yl.

In some embodiments, R₅ is (S)-sec-butyl.

In some embodiments, R₅ is (R)-sec-butyl.

In some embodiments, R₅ is selected from the group consisting of (i), (ii), (iii), (iv), and (v)

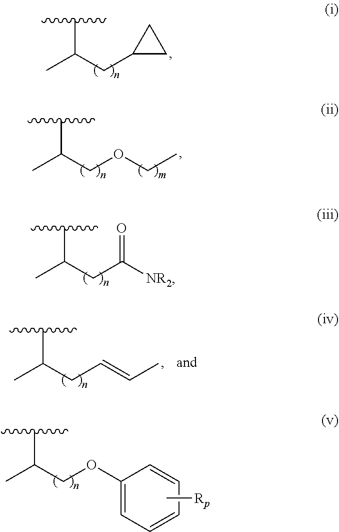

wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

In some embodiments, R₅ is selected from the group consisting of:

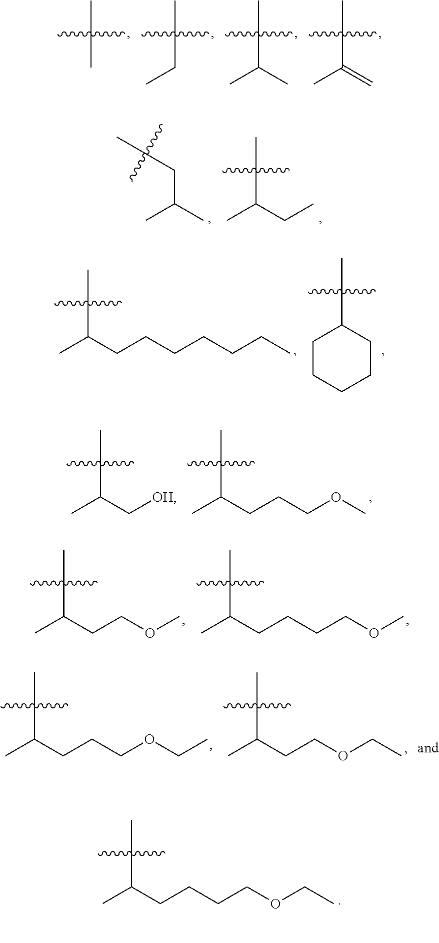

In some embodiments, R₅ is (ii).

In some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl.

In some embodiments, R₅ is (S)-4-methoxybutan-2-yl.

In some embodiments, R₅ is (R)-4-methoxybutan-2-yl.

In some embodiments, R₅ is (S)-5-methoxypentan-2-yl.

In some embodiments, R₅ is (R)-5-methoxypentan-2-yl.

In some embodiments, R₅ is (S)-4-ethoxybutan-2-yl.

In some embodiments, R₅ is (R)-4-ethoxybutan-2-yl.

In some embodiments, Re is hydrogen.

In some embodiments, the aryl hydrocarbon receptor antagonist is a compound represented by formula (II-a)

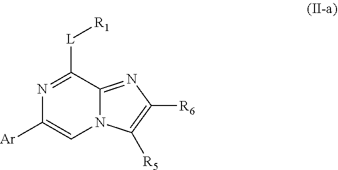

or a pharmaceutically acceptable salt, hydrate, or solvate thereof,

wherein L is a linker selected from the group consisting of —NR₇ₐ(CR₈ₐR_{8b})ₙ—, —O(CR₈ₐR_{8b})ₙ—, —C(O)(CR₈ₐR_{8b})ₙ—, —C(S)(CR₈ₐR_{8b})ₙ—, —S(O)₀₋₂(CR₈ₐR_{8b})ₙ—, —(CR₈ₐR_{8b})ₙ—, —NR₇ₐC(O)(CR₈ₐR_{8b})ₙ—, —NR₇ₐC(S)(CR₈ₐR_{8b})ₙ—, —OC(O)(CR₈ₐR_{8b})ₙ—, —OC(S)(CR₈ₐR_{8b})ₙ—, —C(O)NR₇ₐ(CR₈ₐR_{8b})ₙ—, —C(S)NR₇ₐ(CR₈ₐR_{8b})ₙ—, —C(O)O(CR₈ₐR_{8b})ₙ—, —C(S)O(CR₈ₐR_{8b})ₙ—, —S(O)₂NR₇ₐ(CR₈ₐR_{8b})ₙ—, —NR₇ₐS(O)₂(CR₈ₐR_{8b})ₙ—, —NR₇ₐC(O)NR_{7b}(CR₈ₐR_{8b})ₙ—, and —NR₇ₐC(O)O(CR₈ₐR_{8b})ₙ—, wherein R₇ₐ, R_{7b}, R₈ₐ, and R_{8b} are each independently selected from the group consisting of hydrogen and optionally substituted C1-4 alkyl, and each n is independently an integer from 2 to 6;

R₁ is selected from the group consisting of —S(O)₂NR₉ₐR_{9b}, —NR₉ₐC(O)R_{9b}, —NR₉ₐC(S)R_{9b}, —NR₉ₐC(O)NR₉ₐR_{9c}, —C(O)R₉ₐ, —C(S)R₉ₐ, —S(O)₀₋₂R₉ₐ, —C(O)OR₉ₐ, —C(S)OR₉ₐ, —C(O)NR₉ₐR_{9b}, —C(S)NR₉ₐR₉ₐ, —NR₉ₐS(O)₂R_{9b}, —NR₉ₐC(O)OR_{9b}, —OC(O)CR₉ₐR_{9b}, —OC(S)CR₉ₐR_{9b}R_{9c}, optionally substituted aryl, optionally substituted heteroaryl, optionally substituted cycloalkyl, and optionally substituted heterocycloalkyl, wherein R₉ₐ, R_{9b}, and R_{9c} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl (for example, R₁ may be selected from the group consisting of phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted, for example, with from 1 to 3 substituents independently selected from the group consisting of cyano, hydroxy, C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl);

Ar is selected from the group consisting of optionally substituted monocyclic aryl and heteroaryl, such as optionally substituted thiophenyl, furanyl, 1H-benzoimidazolyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl;

R₃ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and

R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

or a salt thereof.

In some embodiments, Ar is pyridin-3-yl, wherein the pyridin-3-yl is optionally substituted at C5, for example, with a substituent selected from the group consisting of ethoxycarbonyl, methoxy, cyano, methyl, methylsulfonyl, fluoro, chloro, trifluoromethyl, ethynyl, and cyclopropyl.

In some embodiments, the aryl hydrocarbon receptor antagonist is a compound represented by formula (II-b)

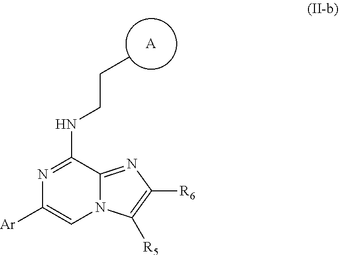

or a pharmaceutically acceptable salt, hydrate, or solvate thereof,

wherein A is an optionally substituted ring system selected from the group consisting of phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C1-4 alkyl, C₁₋₄ alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

Ar is selected from the group consisting of optionally substituted monocyclic aryl and heteroaryl, such as optionally substituted thiophenyl, furanyl, 1H-benzoimidazolyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl;

R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and

R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

or a salt thereof.

In some embodiments, A is selected from the group consisting of phenyl, phenol-4-yl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, and 2-oxo-2,3-dihydro-1H-benzo[d]imidazol-5-yl.

In some embodiments, A is selected from the group consisting of phenol-4-yl and 1H-indol-3-yl.

In some embodiments, the aryl hydrocarbon receptor antagonist is a compound represented by formula (II-c)

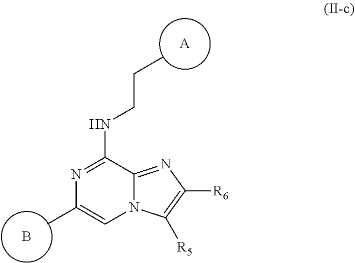

or a pharmaceutically acceptable sa, hydrate, or solvate thereof,

wherein A is an optionally substituted ring system selected from the group consisting of phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C1-4 alkyl, C₁₋₄ alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

B is an optionally substituted ring system selected from the group consisting of thiophenyl, furanyl, 1H-benzoimidazolyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl, wherein the thiophenyl, furanyl, 1H-benzoimidazolyl, isoquinolinyl, 1H-imidazopyridinyl, benzothiophenyl, pyrimidinyl, pyridinyl, 1H-imidazolyl, pyrazinyl, pyridazinyl, 1H-pyrrolyl, or thiazolyl is optionally substituted with from 1 to 3 substituents independently selected from the group consisting of cyano, hydroxy. C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄alkyl;

R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and

R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

or a salt thereof.

In some embodiments, B is pyridin-3-yl, wherein the pyridin-3-yl is optionally substituted at C5, for example, with a substituent selected from the group consisting of ethoxycarbonyl, methoxy, cyano, methyl, methylsulfonyl, fluoro, chloro, trifluoromethyl, ethynyl, and cyclopropyl.

In some embodiments, the aryl hydrocarbon receptor antagonist is a compound represented by formula (II-d)

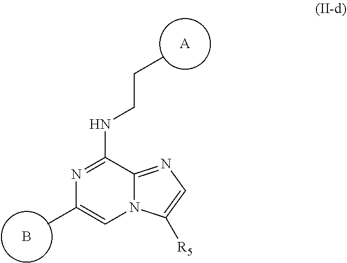

or a pharmaceutically acceptable salt, hydrate, or solvate thereof,

wherein A is an optionally substituted ring system selected from the group consisting of phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C1-4alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

B is an optionally substituted ring system selected from the group consisting of thiophenyl, furanyl, 1H-benzoimidazolyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl, wherein the thiophenyl, furanyl, 1H-benzoimidazolyl, isoquinolinyl, 1H-imidazopyridinyl, benzothiophenyl, pyrimidinyl, pyridinyl, 1H-imidazolyl, pyrazinyl, pyridazinyl, 1H-pyrrolyl, or thiazolyl is optionally substituted with from 1 to 3 substituents independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and

R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl;

or a salt thereof.

In some embodiments, the aryl hydrocarbon receptor antagonist is a compound represented by formula (II-e)

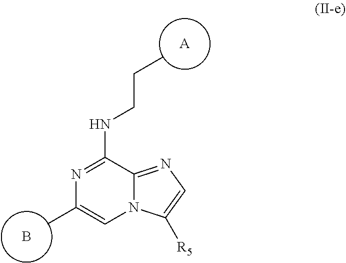

or a pharmaceutically acceptable salt, hydrate, or solvate thereof,

wherein A is an optionally substituted ring system selected from the group consisting of phenyl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, and 2-oxo-2,3-dihydro-1H-benzo[d]imidazol-5-yl, wherein the phenyl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, or 2-oxo-2,3-dihydro-1H-benzo[d]imidazol-5-yl is optionally substituted with from 1 to 3 substituents independently selected from the group consisting of cyano, hydroxy, C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

B is an optionally substituted ring system selected from the group consisting of thiophen-2-yl, thiophen-3-yl, furan-3-yl, 1H-benzo[d]imidazol-1-yl, isoquinolin-4-yl, 1H-imidazo[4,5-b]pyridin-1-yl, imidazo[1,2-a]pyridin-3-yl, benzo[b]thiophen-3-yl, pyrimidin-5-yl, pyridin-2-yl, pyridin-3-yl, pyridin-4-yl, 1H-imidazol-1-yl, pyrazin-2-yl, pyridazin-4-yl, 1H-pyrrol-2-yl and thiazol-5-yl, wherein the thiophen-2-yl, thiophen-3-yl, furan-3-yl, 1H-benzo[d]imidazol-1-yl, isoquinolin-4-yl, 1H-imidazo[4,5-b]pyridin-1-yl, benzo[b]thiophen-3-yl, pyrimidin-5-yl, pyridin-2-yl, pyridin-3-yl, pyridin-4-yl, 1H-imidazol-1-yl, pyrazin-2-yl, pyridazin-4-yl, 1H-pyrrol-2-yl, or thiazol-5-yl is optionally substituted with from 1 to 3 substituents independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and

R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4alkyl, or R₅ is selected from the group consisting of (i), (ii), (ii), (iv), and (v)

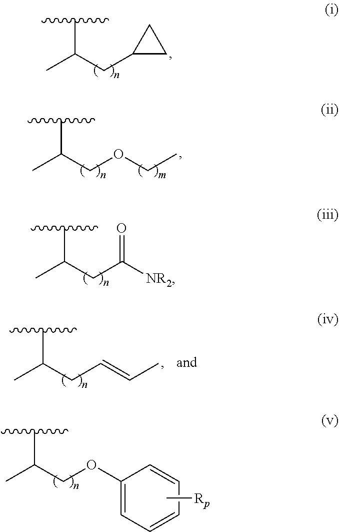

wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl;

In some embodiments, R₅ is selected from the group consisting of:

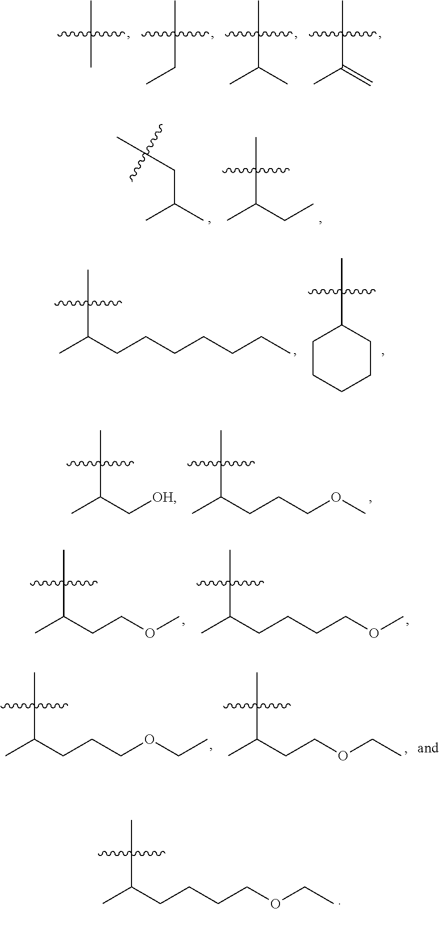

in some embodiments, R₅ is (ii);

in some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl;

or a salt thereof.

In some embodiments, the aryl hydrocarbon receptor antagonist is a compound represented by formula (II-f)

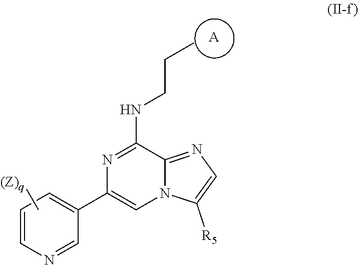

or a pharmaceutically acceptable salt, hydrate, or solvate thereof,

wherein A is an optionally substituted ring system selected from the group consisting of phenol-4-yl and 1H-indol-3-yl;

q is an integer from 0 to 4;

each Z is independently a substituent selected from the group consisting of C1-4 alkyl, halo, halo-substituted-C1-4 alkyl, C1-4 alkenyl, C1-4 alkynyl, C2-6 cycloalkyl. C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄alkyl; and

R₅ is selected from the group consisting of isopropyl, methyl, ethyl, prop-1-en-2-yl, isobutyl, cyclohexyl, sec-butyl, (S)-sec-butyl, (R)-sec-butyl, 1-hydroxypropan-2-yl, (S)-1-hydroxypropan-2-yl, (R)-1-hydroxypropan-2-yl, and nonan-2-yl, or R₅ is selected from the group consisting of (i), (ii), (iii), (iv), and (v)

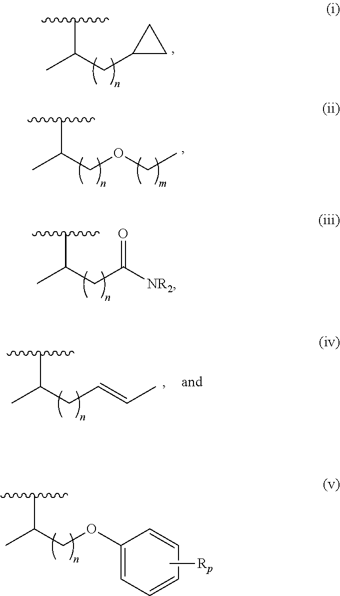

wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl;

In some embodiments, R₅ is selected from the group consisting of:

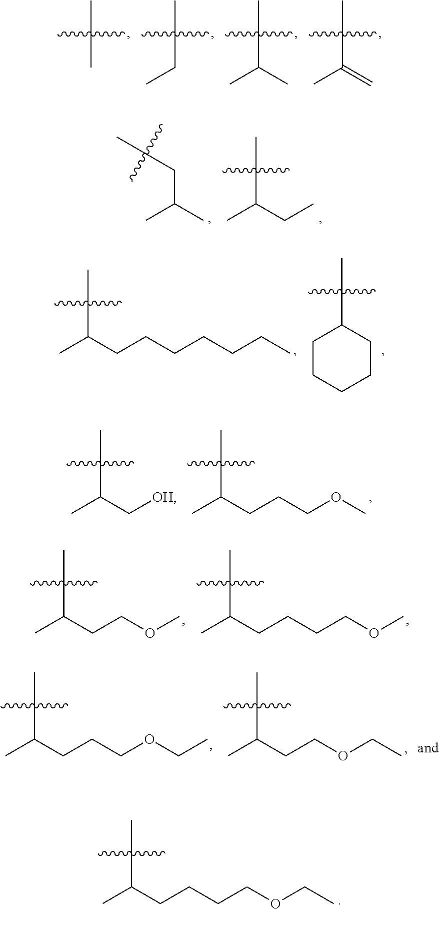

in some embodiments, R₅ is (ii);

in some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl;

or a salt thereof.

In some embodiments, each Z is independently a substituent selected from the group consisting of ethoxycarbonyl, methoxy, cyano, methyl, methylsulfonyl, fluoro, chloro, trifluoromethyl, ethynyl, and cyclopropyl.

In some embodiments, the aryl hydrocarbon receptor antagonist is a compound represented by formula (II-g)

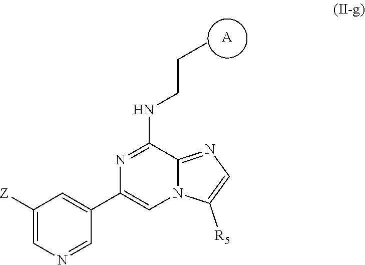

or a pharmaceutically acceptable salt, hydrate, or solvate thereof,

wherein A is an optionally substituted ring system selected from the group consisting of phenol-4-yl and 1H-indol-3-yl;

Z is a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and

R₅ is selected from the group consisting of isopropyl, methyl, ethyl, prop-1-en-2-yl, isobutyl, cyclohexyl, sec-butyl, (S)-sec-butyl, (R)-sec-butyl, 1-hydroxypropan-2-yl, (S)-1-hydroxypropan-2-yl, (R)-1-hydroxypropan-2-yl, and nonan-2-yl, or R₅ is selected from the group consisting of (i), (ii), (iii), (iv), and (v)

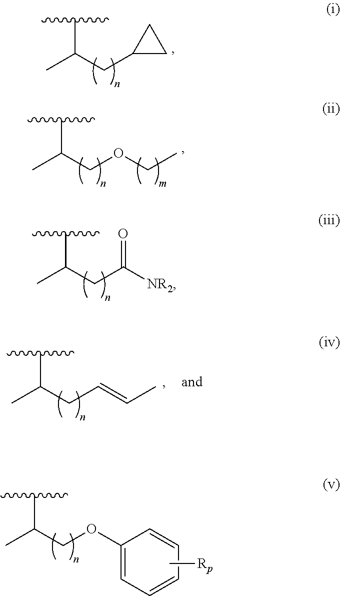

wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄alkyl;

In some embodiments, R₅ is selected from the group consisting of:

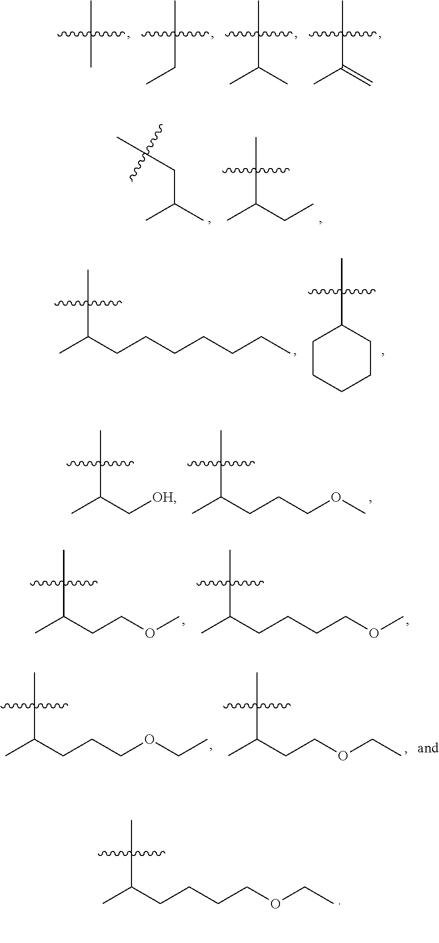

in some embodiments, R₅ is (ii);

in some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl;

or a salt thereof.

In some embodiments, the aryl hydrocarbon receptor antagonist is a compound represented by formula (II-h)

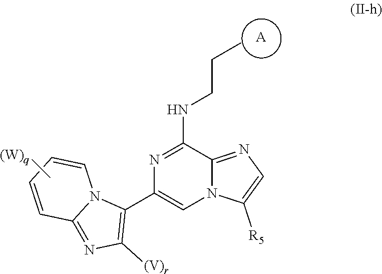

or a pharmaceutically acceptable salt, hydrate, or solvate thereof,

wherein A is an optionally substituted ring system selected from the group consisting of phenol-4-yl and 1H-indol-3-yl;

q is an integer from 0 to 4;

r is 0 or 1;

W and V are each independently a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄alkyl; and

R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4alkyl, or R₅ is selected from the group consisting of (i), (ii), (iii), (iv), and (v)

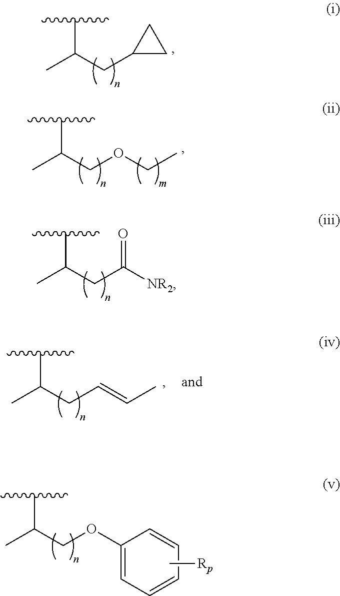

wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl;

In some embodiments, R₅ is selected from the group consisting of:

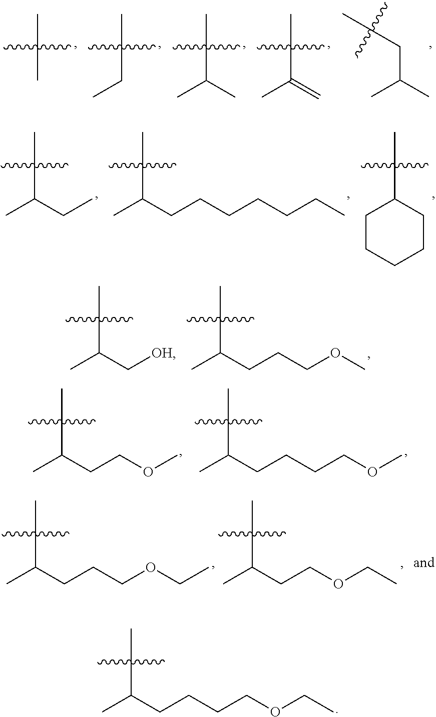

in some embodiments, R₅ is (ii);

in some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl;

or a salt thereof.

In some embodiments, the aryl hydrocarbon receptor antagonist is a compound represented by formula (II-i)

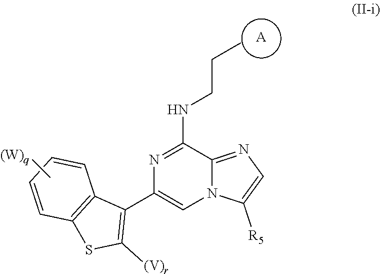

or a pharmaceutically acceptable salt, hydrate, or solvate thereof,

wherein A is an optionally substituted ring system selected from the group consisting of phenol-4-yl and 1H-indol-3-yl;

q is an integer from 0 to 4;

r is 0 or 1;

W and V are each independently a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄alkyl; and

R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4alkyl, or R₅ is selected from the group consisting of (i, (ii), (iii), (iv), and (v)

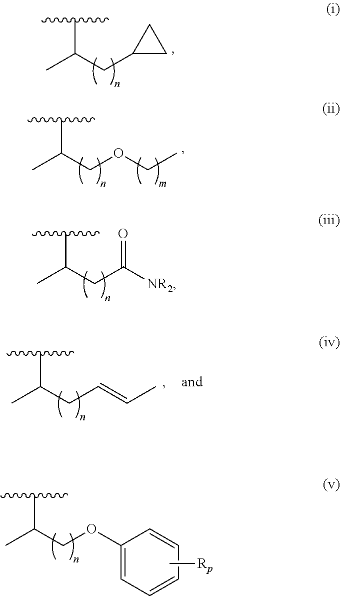

wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl;

In some embodiments, R₅ is selected from the group consisting of:

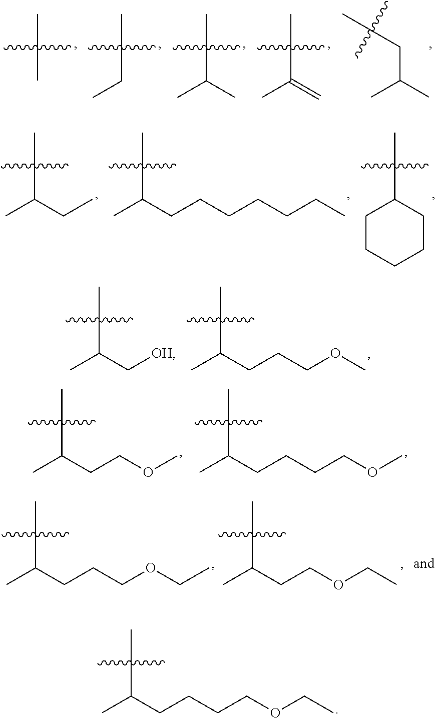

in some embodiments, R₅ is (ii);

in some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl;

or a salt thereof.

In some embodiments, the aryl hydrocarbon receptor antagonist is a compound represented by formula (II-j)

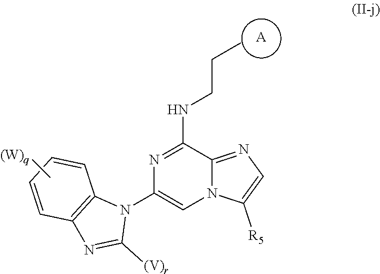

or a pharmaceutically acceptable salt, hydrate, or solvate thereof,

wherein A is an optionally substituted ring system selected from the group consisting of phenol-4-yl and 1H-indol-3-yl;

q is an integer from 0 to 4;

r is 0 or 1;

W and V are each independently a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{1b}1 are each independently selected from the group consisting of hydrogen and C₁₋₄alkyl; and

R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4alkyl, or R₅ is selected from the group consisting of (i), (ii), (iii), (iv), and (v)

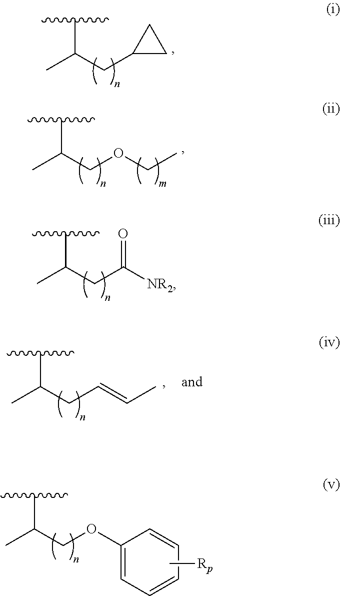

wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl;

In some embodiments, R₅ is selected from the group consisting of:

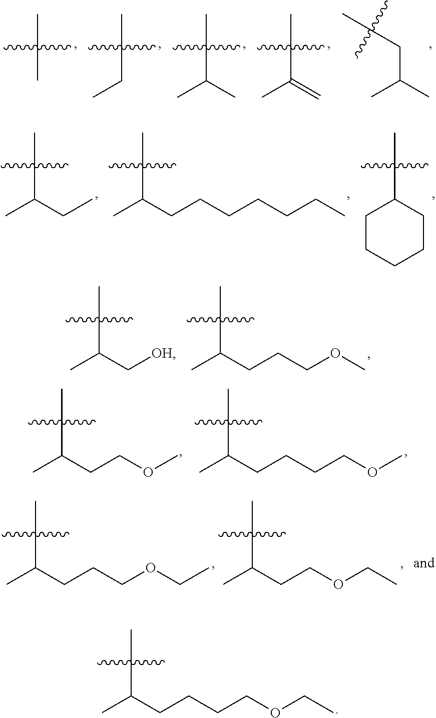

in some embodiments, R₅ is (ii);

in some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl;

or a salt thereof.

In some embodiments, the aryl hydrocarbon receptor antagonist is a compound represented by formula (II-k)

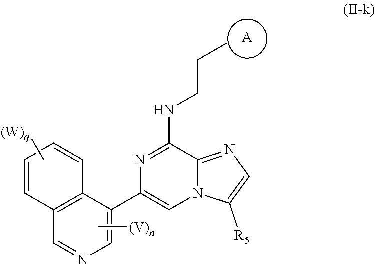

or a pharmaceutically acceptable salt, hydrate, or solvate thereof,

wherein A is an optionally substituted ring system selected from the group consisting of phenol-4-yl and 1H-indol-3-yl;

q is an integer from 0 to 4;

r is 0 or 1;

W and V are each independently a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{1b}1 are each independently selected from the group consisting of hydrogen and C₁₋₄alkyl; and

R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4alkyl, or R₅ is selected from the group consisting of (i), (ii), (iii), (iv), and (v)

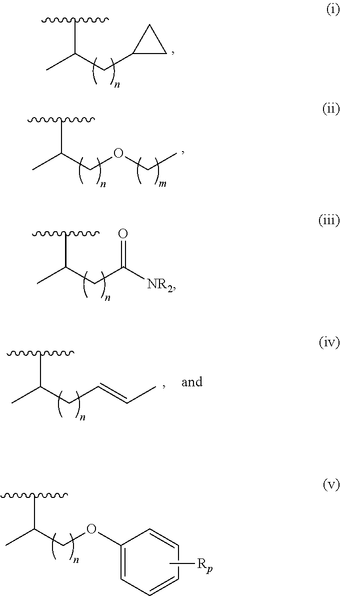

wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl;

In some embodiments, R₅ is selected from the group consisting of:

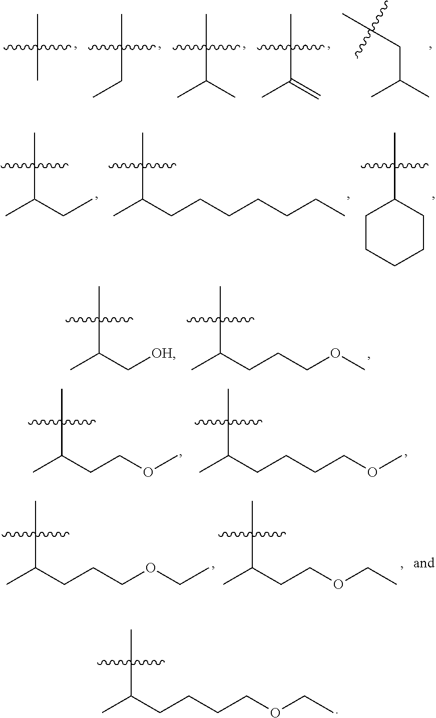

in some embodiments, R₅ is (ii);

in some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl;

or a salt thereof.

In some embodiments, the compound is compound (12)

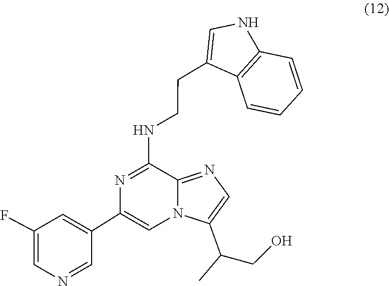 [SMILES: CC(CO)c1cnc2c(NCCc3c[nH]c4ccccc34)nc(-c3cncc(F)c3)cn12]

or a salt thereof.

In some embodiments, the compound is compound (13)

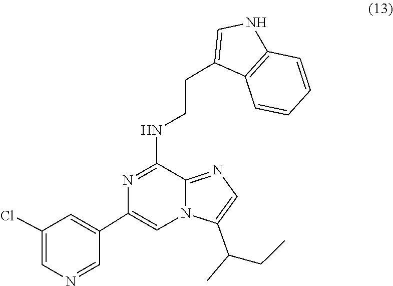 [SMILES: CCC(C)c1cnc2c(NCCc3c[nH]c4ccccc34)nc(-c3cncc(Cl)c3)cn12]

or a salt thereof.

In some embodiments, the compound is compound (14)

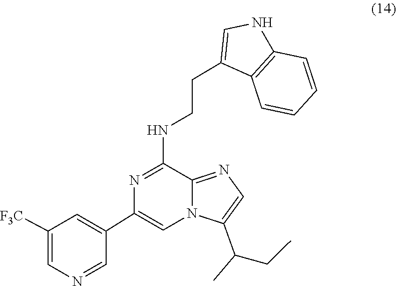 [SMILES: CCC(C)c1cnc2c(NCCc3c[nH]c4ccccc34)nc(-c3cncc(C(F)(F)F)c3)cn12]

or a salt thereof.

In some embodiments, the compound is compound (15)

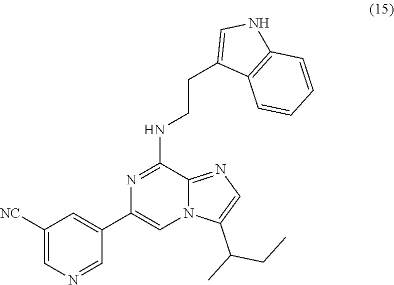 [SMILES: CCC(C)c1cnc2c(NCCc3c[nH]c4ccccc34)nc(-c3cncc(C#N)c3)cn12]

or a salt thereof.

In some embodiments, the compound is compound (16)

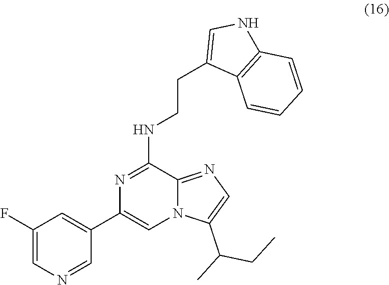 [SMILES: CCC(C)c1cnc2c(NCCc3c[nH]c4ccccc34)nc(-c3cncc(F)c3)cn12]

or a salt thereof.

In some embodiments, the compound is compound (17)

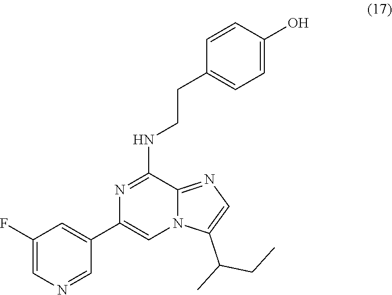 [SMILES: CCC(C)c1cnc2c(NCCc3ccc(O)cc3)nc(-c3cncc(F)c3)cn12]

or a salt thereof.

In some embodiments, the compound is compound (18)

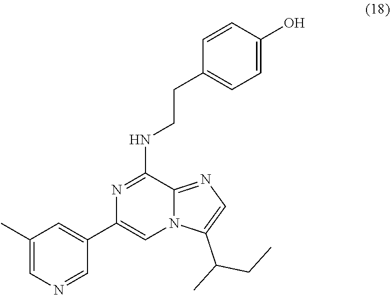 [SMILES: CCC(C)c1cnc2c(NCCc3ccc(O)cc3)nc(-c3cncc(C)c3)cn12]

or a salt thereof.

In some embodiments, the compound is compound (19)

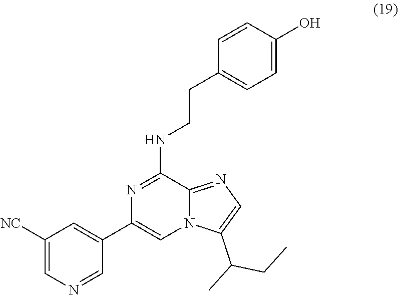 [SMILES: CCC(C)c1cnc2c(NCCc3ccc(O)cc3)nc(-c3cncc(C#N)c3)cn12]

or a salt thereof.

In some embodiments the compound is compound (20)

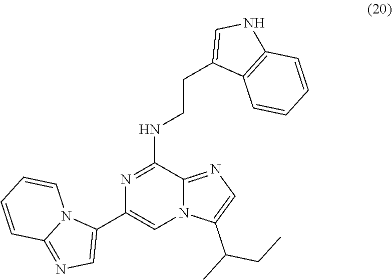 [SMILES: CCC(C)c1cnc2c(NCCc3c[nH]c4ccccc34)nc(-c3cnc4ccccn34)cn12]

or a salt thereof.

In some embodiments, the compound is compound (21)

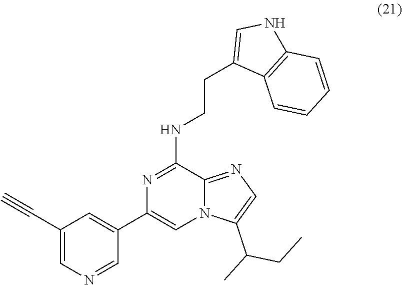 [SMILES: C#Cc1cncc(-c2cn3c(C(C)CC)cnc3c(NCCc3c[nH]c4ccccc34)n2)c1]

or a salt thereof.

In some embodiments, the compound is compound (22)

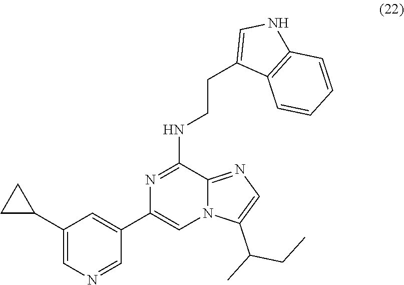 [SMILES: CCC(C)c1cnc2c(NCCc3c[nH]c4ccccc34)nc(-c3cncc(C4CC4)c3)cn12]

or a salt thereof.

In some embodiments, the compound is compound (24)

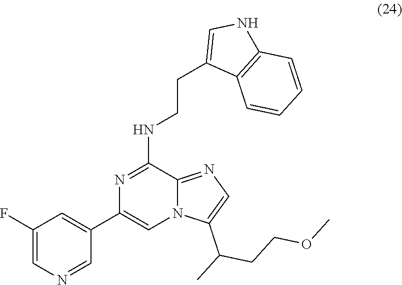 [SMILES: COCCC(C)c1cnc2c(NCCc3c[nH]c4ccccc34)nc(-c3cncc(F)c3)cn12]

or a salt thereof.

In some embodiments, the compound is compound (27)

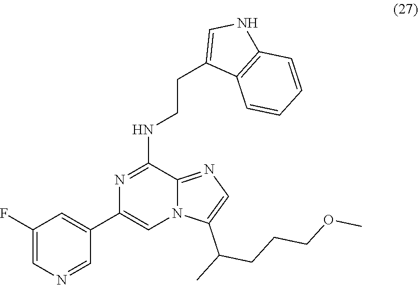 [SMILES: COCCCC(C)c1cnc2c(NCCc3c[nH]c4ccccc34)nc(-c3cncc(F)c3)cn12]

or a salt thereof.

In some embodiments, the compound is compound (28)

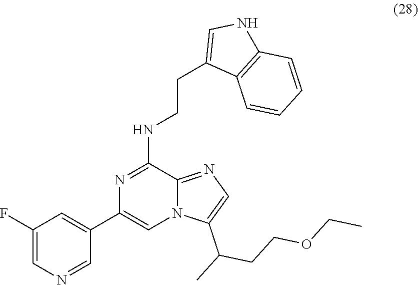 [SMILES: CCOCCC(C)c1cnc2c(NCCc3c[nH]c4ccccc34)nc(-c3cncc(F)c3)cn12]

or a salt thereof.

Where the number of any given substituent is not specified, there may be one or more substituents present. For example, “halo-substituted C1-4 alkyl” may include one or more of the same or different halogens.

When the compounds described herein contain olefinic double bonds or other centers of geometric asymmetry, and unless specified otherwise, it is intended that the compounds include both E and Z geometric isomers. Likewise, all tautomeric forms of carbonyl-containing compounds are also intended to be included.

It is to be understood that the compounds provided herein may contain chiral centers. Such chiral centers may be of either the (R) or (S) configuration, or may be a mixture thereof. Thus, the compounds provided herein may be enantiomerically pure, or may be stereoisomeric or diastereomeric mixtures. As such, one of skill in the art will recognize that administration of a compound in its (R) form is equivalent, for compounds that undergo epimerization in vivo, to administration of the compound in its (S) form.

Compounds described herein include, but are not limited to, those set forth above, as well as any of their isomers, such as diastereomers and enantiomers, as well as salts, esters, amides, thioesters, solvates, and polymorphs thereof, as well as racemic mixtures and pure isomers of the compounds set forth above.

### Synthesis

### Substituent Protecting Groups

The synthesis of aryl hydrocarbon receptor antagonists described herein may involve the selective protection and deprotection of alcohols, amines, ketones, sulfhydryls or carboxyl functional groups of a precursor. For example, commonly used protecting groups for amines include carbamates, such as tert-butyl, benzyl, 2,2,2-trichloroethyl, 2-trimethylsilylethyl, 9-fluorenylmethyl, allyl, and m-nitrophenyl. Other commonly used protecting groups for amines include amides, such as formamides, acetamides, trifluoroacetamides, sulfonamides, trifluoromethanesulfonyl amides, trimethylsilylethanesulfonamides, and tert-butylsulfonyl amides. Examples of commonly used protecting groups for carboxyls include esters, such as methyl, ethyl, tert-butyl, 9-fluorenylmethyl, 2-(trimethylsilyl)ethoxy methyl, benzyl, diphenylmethyl, O-nitrobenzyl, ortho-esters, and halo-esters. Examples of commonly used protecting groups for alcohols include ethers, such as methyl, methoxymethyl, methoxyethoxymethyl, methylthiomethyl, benzyloxymethyl, tetrahydropyranyl, ethoxyethyl, benzyl, 2-napthylmethyl, O-nitrobenzyl, P-nitrobenzyl, P-methoxybenzyl, 9-phenylxanthyl, trityl (including methoxy-trityls), and silyl ethers. Examples of commonly used protecting groups for sulfhydryls include many of the same protecting groups used for hydroxyls. In addition, sulfhydryls can be protected in a reduced form (e.g., as disulfides) or an oxidized form (e.g., as sulfonic acids, sulfonic esters, or sulfonic amides). Protecting groups can be chosen such that selective conditions (e.g., acidic conditions, basic conditions, catalysis by a nucleophile, catalysis by a Lewis acid, or hydrogenation) are required to remove each, exclusive of other protecting groups in a compound. The conditions required for the addition of protecting groups to amine, alcohol, sulfhydryl, and carboxyl functionalities and the conditions required for their removal are provided in detail, for example, in T. W. Green and P. G. M. Wuts, Protective Groups in Organic Synthesis (2^{nd} Ed.). John Wiley & Sons, 1991 and P. J. Kocienski, Protecting Groups, Georg Thieme Verlag, 1994.

### Exemplary Synthetic Methods

Aryl hydrocarbon receptor antagonists represented by formula (I) or (II) may be synthesized, for instance, by way of a palladium-catalyzed coupling reaction, such as a process depicted in Scheme 1, below.

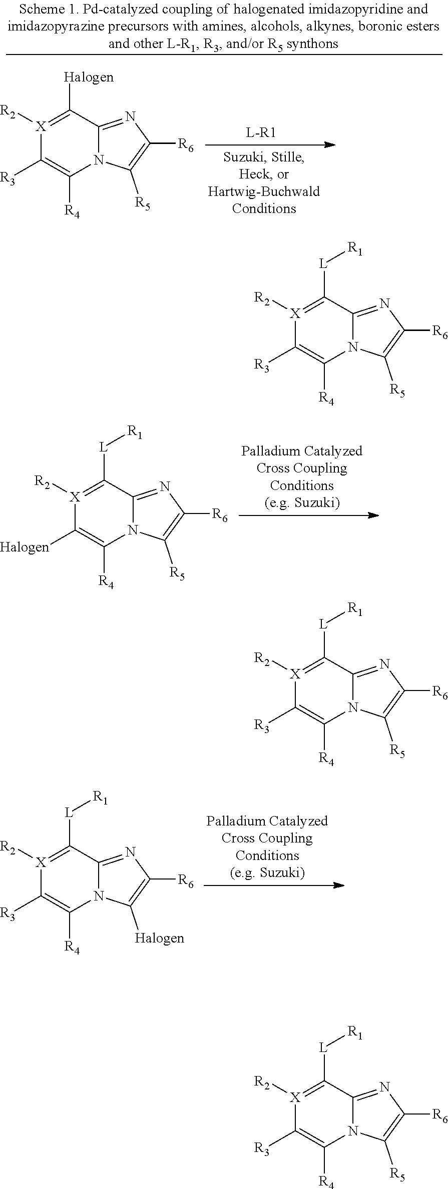

wherein X is C or N, with the proviso that R₂ is absent when X is N;

L is selected from the group consisting of —NR₇ₐ(CR₈ₐR_{8b})ₙ—, —O(CR₈ₐR_{8b})ₙ—, —S(O)₀₋₂(CR₈ₐR_{8b})—, and —(CR₈ₐR_{8b})ₙ—, wherein R₇ₐ, R₈ₐ, and R_{8b} are each independently selected from the group consisting of hydrogen and optionally substituted C1-4 alkyl, and each n is independently an integer from 2 to 6;

R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉ₐ, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)CR₉ₐR_{9b}, —OC(S)CR₉ₐR_{9b}R_{9c}, optionally substituted aryl, optionally substituted heteroaryl, optionally substituted cycloalkyl, and optionally substituted heterocycloalkyl, wherein R₉ₐ, R_{9b}, and R_{9c} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

R₂ is selected from the group consisting of hydrogen and optionally substituted C1-4 alkyl;

R₃ is selected from the group consisting of optionally substituted aryl, optionally substituted heteroaryl, optionally substituted cycloalkyl, and optionally substituted heterocycloalkyl;

R₄ is selected from the group consisting of hydrogen and optionally substituted C1-4 alkyl;

R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and

R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

For instance, when an organoboron species is employed as a synthon such as, for example, R₃—B(pin) and/or R₅—B(pin), Suzuki or Suzuki-Miyaura reaction conditions may be used to join the halogenated imidazopyridine or imidazopyrazine precursor with the organoboron R₃ or R₅ synthon species in the presence of a Pd catalyst, such as, for example Pd(dppf)Cl₂.

For instance, when L is an amino- or hydroxy-containing linker, Hartwig-Buchwald conditions may be used to join the halogenated precursor with the amine or alcohol in the presence of a Pd catalyst, as shown in Scheme 2, below.

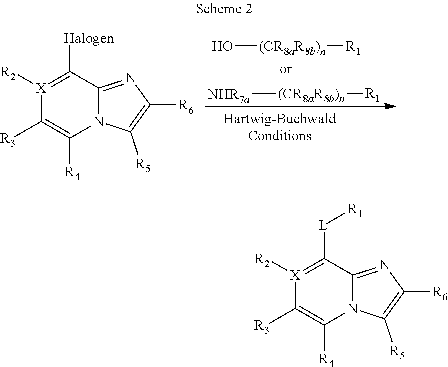

Exemplary Hartwig-Buchwald conditions include the use of a Pd catalyst, such as Pd₂(dba)₃, in the presence of dicyclohexylphosphino-2′-(N,N-dimethylamino)biphenyl, tBuONa, dioxane, 120° C., microwave irradiation. Hartwig-Buchwald reaction conditions are known in the art and are described, for instance, in Bailey et al., Bioorganic and Medicinal Chemistry Letters 19:3602-3606 (2009), the disclosure of which is incorporated herein by reference as it pertains to conditions useful for the Hartwig-Buchwald amination or etherification.

When L is a thiol, nucleophilic aromatic substitution conditions may be employed to join the linker to a halogenated precursor, as shown in Scheme 3, below.

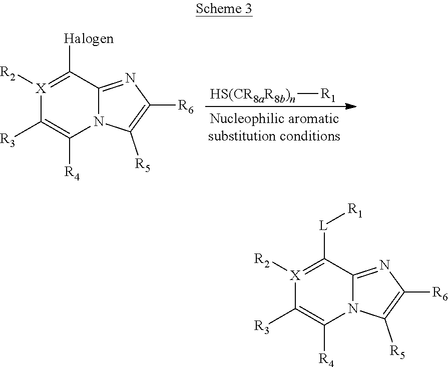

Nucleophilic aromatic substitution conditions include the use of a base to deprotonate the thiol shown in the above linker-R₁ pair. This reactive modality is particularly useful when the halogenated aryl precursor is activated by the presence of one or more electron-withdrawing substituents, (such as nitro, cyano, trifluoromethyl, trichloromethyl, and the like) and/or when X is nitrogen.

Additional amine, hydroxyl, and thiol arylation techniques that may be used to produce the compounds described herein include those described in Burke, A. J. and Marques, C. S. (eds) (2014) Amine, Phenol, Alcohol, and Thiol Arylation, in Catalytic Arylation Methods: From the Academic Lab to Industrial Processes, Wiley-VCH Verlag GmbH & Co. KGaA, Weinheim, Germany, the disclosure of which is incorporated herein by reference as it pertains to processes for chemical synthesis.

A Pd-catalyzed Heck reaction can be used to join olefinic linkers to a halogenated aryl precursor, as shown in Scheme 4, below.

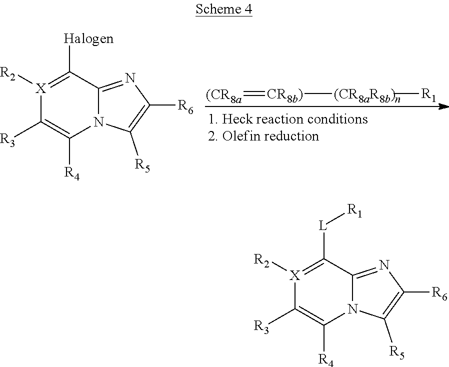

wherein “═” denotes a carbon-carbon double bond, which can subsequently be reduced to yield a saturated alkylene linker using olefin reduction methods known in the art.

For instances in which L is selected from the group consisting of —C(O)(CR₈ₐR_{8b})ₙ—, —C(S)(CR₈ₐR₈ₐ)ₙ—, —NR₇ₐC(O)(CR₈ₐR_{8b})ₙ, —NR₇ₐC(S)(CR₈ₐR_{8b})ₙ—, —OC(O)(CR₈ₐR_{8b})ₙ—, —OC(S)(CR₈ₐR_{8b})ₙ—, —C(O)NR₇ₐ(CR₈ₐR_{8b})ₙ—, —C(S)NR₇ₐ(CR₈ₐR_{8b})ₙ—, —C(O)O(CR₈ₐR_{8b})ₙ—, —C(S)O(CR₈ₐR_{8b})ₙ—, —S(O)₂NR₇ₐ(CR₈ₐR_{8b})ₙ—, —NR₇ₐS(O)₂(CR₈ₐR_{8b})ₙ—, —NR₇ₐC(O)NR_{7b}(CR₈ₐR_{8b})ₙ—, and —NR₇ₐC(O)O(CR₈ₐR₈ₐ)ₙ—, techniques such as acylation, sulfonamidation, and thionation reactions can be employed in order to produce compounds represented by formulas (I) and (II). Exemplary acylation, sulfonamidation, and thionation processes that may be used to synthesize the aryl hydrocarbon receptor antagonists described herein are depicted in Schemes 5-7, below.

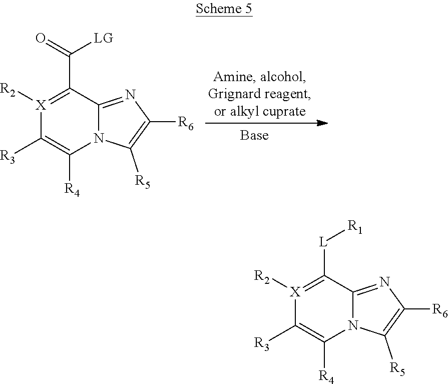

In Scheme 5, LG denotes a nucleofugal leaving group, such as a halogen (for instance, chlorine or bromine), a sulfonate (for instance, tosylate, brosylate, triflate, mesylate, and the like), and other leaving groups known in the art. In this way, linkers (“L”) containing an amide, ester, ketone, urea, carbamate, or the like in which the carbonyl carbon is bound directly to the imidazopyridine or imiadzopyrazine ring system can be synthesized.

Similarly, to linkers (“L”) containing a thioketone, thioamide, thioester, and the like can be synthesized by reacting the corresponding ketone, amide, or ester with a thionating reagent, as shown in Scheme 6, below.

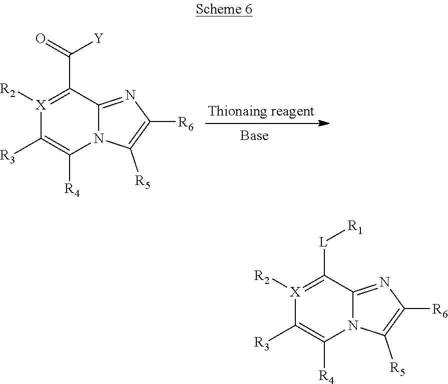

wherein C(O)Y denotes an amide, ester, ketone, or the like.

Exemplary thionating reagents are known in the art and include, for instance, Lawesson's reagent, which is described, for example, Jesberger, et al., Synthesis 13:1929-1258 (2003), the disclosure of which is incorporated herein by reference as it pertains to thionation techniques.

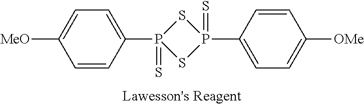 [SMILES: COc1ccc(P2(=S)SP(=S)(c3ccc(OC)cc3)S2)cc1]

When the linker (“L”) contains a sulfonamide moiety bound to the imidazopyridine or imadazopyrazine ring system, for instance, at the sulfur or nitrogen of the sulfonamide functionality, sulfonamidation techniques known in the art can be used to produce the corresponding compound of formula (I) or (II). An exemplary sulfonamidation process is depicted in Scheme 7, below.

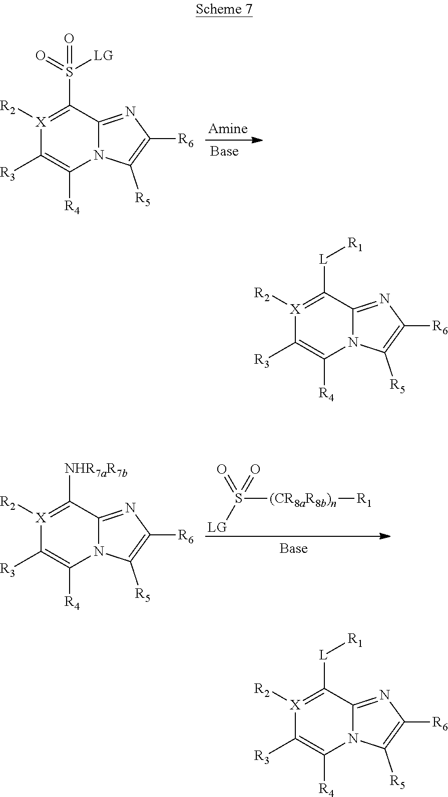

In Scheme 7, LG denotes a nucleofugal leaving group, such as a halogen (for instance, chlorine or bromine), a sulfonate (for instance, tosylate, brosylate, triflate, mesylate, and the like), and other leaving groups known in the art. In this way, linkers (“L”) containing a sulfonamide in which the sulfur or nitrogen of the sulfonamide moiety is bound directly to the imidazopyridine or imiadzopyrazine ring system can be synthesized.

### Methods of Use

In some aspects, the present disclosure provides a method of modulating the activity of an aryl hydrocarbon receptor, comprising administering to a subject in need thereof an effective amount of a compound (e.g., an aryl hydrocarbon receptor antagonist) described herein.

In some aspects, the present disclosure provides a method of treating or preventing a disease or disorder, comprising administering to a subject in need thereof an effective amount of a compound (e.g., an aryl hydrocarbon receptor antagonist) described herein.

In some aspects, the present disclosure provides a compound (e.g., an aryl hydrocarbon receptor antagonist) described herein for use in modulating the activity of an aryl hydrocarbon receptor in a subject in need thereof.

In some aspects, the present disclosure provides a compound (e.g., an aryl hydrocarbon receptor antagonist) described herein for use in treating or preventing a disease or disorder in a subject in need thereof.

In some aspects, the present disclosure provides use of a compound (e.g., an aryl hydrocarbon receptor antagonist) described herein in the preparation or manufacture of a medicament for modulating the activity of an aryl hydrocarbon receptor in a subject in need thereof.

In some aspects, the present disclosure features use of a compound (e.g., an aryl hydrocarbon receptor antagonist) described herein in the preparation or manufacture of a medicament for treating or preventing a disease or disorder in a subject in need thereof.

In some aspects, the present disclosure provides a pharmaceutical composition or kit described herein for use in modulating the activity of an aryl hydrocarbon receptor in a subject in need thereof.

In some aspects, the present disclosure provides a pharmaceutical composition or kit described herein for use in treating or preventing a disease or disorder in a subject in need thereof.

In some embodiments, the disease or disorder is characterized by the production of an aryl hydrocarbon receptor agonist.

In some embodiments, the disease or disorder is a cancer, a cancerous condition, or a tumor.

In some embodiments, the tumor is an invasive tumor.

In some embodiments, the cancer is a breast cancer, squamous cell cancer, lung cancer, a cancer of the peritoneum, a hepatocellular cancer, a gastric cancer, a pancreatic cancer, a glioblastoma, a cervical cancer, an ovarian cancer, a liver cancer, a bladder cancer, a hepatoma, a colon cancer, a colorectal cancer, an endometrial or uterine carcinoma, a salivary gland carcinoma, a kidney or renal cancer, a prostate cancer, a vulval cancer, a thyroid cancer, a head and neck cancer, a B-cell lymphoma, a chronic lymphocytic leukemia (CLL); an acute lymphoblastic leukemia (ALL), a Hairy cell leukemia, or a chronic myeloblastic leukemia.

In some embodiments, the method further comprises administering one or more additional anti-cancer therapies.

In some embodiments, the methods of the present disclosure may comprise contacting a compound or aryl hydrocarbon receptor antagonist as described herein with a tumor activity system, wherein said tumor activity system may comprise (i) a tumor cell and/or (ii) a mixture comprising one or more extracellular matrix components. In some embodiments, the tumor activity being measured in the tumor activity assay system may be tumor cell proliferation or tumor cell invasiveness.

Without wishing to be bound by theory, it has been suggested that the enzymes IDO1 and TDO2 may be involved in a pathway that produces aryl hydrocarbon agonists, which suppress the immune system and enable a tumor to evade eradication by the immunes system. Anti-cancer and/or anti-tumor activity is thus suggested by an immune-oncology mechanism whereby administering aryl hydrocarbon receptor antagonists, such as those disclosed herein, may counteract the immunosuppressive effects of aryl hydrocarbon agonist, thereby allowing a patient's immune system to recognize and/or eradicate a tumor.

In some embodiments, the anticancer activity of the small molecule aryl hydrocarbon receptor antagonists of Formula (I) or Formula (II), compositions thereof, methods and uses thereof described herein may be established in a cell line model, tumor cell line model, and/or an animal model. Exemplary cell lines include, but are not limited to, human breast cancer cells (MCF-7, MDA-468, and SK-Br-3), human liver carcinoma cells (Hep-G2), human colon adinocarcinoma cells (Colo320 D-M), human acute promylocytic leukemia cells (HL-60), mouse sarcoma cells (Sarcoma 180). mouse melanoma cells (C57/B1/6J). Cells may be maintained or grown in suitable media and contacted and/or incubated with various concentrations of the small molecule aryl hydrocarbon receptor antagonists of Formula (I) or Formula (II) and compositions thereof as described herein. Morphological changes in the cells and cell proliferation activity may be observed and demonstrate the anti-cancer activity of the aryl hydrocarbon receptor antagonists of the present disclosure.

In some embodiments the small molecule aryl hydrocarbon receptor antagonists of Formula (I) or Formula (II), compositions thereof, methods and uses thereof described herein may produce marked anti-cancer effects in a human subject without causing significant toxicities or adverse effects. The efficacy of the treatments described herein can be measured by various parameters commonly used in evaluating cancer treatments, including but not limited to, tumor regression, tumor weight or size shrinkage, reduction in rate of tumor growth, the presence or the size of a dormant tumor, the presence or size of metastases or micrometastases, degree of tumor or cancer invasiveness, size or number of the blood vessels, time to progression, duration of survival, progression free survival, overall response rate, duration of response, and quality of life. For example, tumor shrinkage of greater than 50% in a 2-dimensional analysis may be a cut-off for declaring a response.

In some embodiments, the small molecule aryl hydrocarbon receptor antagonists of Formula (I) or Formula (II), compositions thereof, methods and uses thereof described herein may be used to cause inhibition of metastatic spread without shrinkage of the primary tumor, or may simply exert a tumoristatic effect. In the case of cancers, the small molecule aryl hydrocarbon receptor antagonists of Formula (I) or Formula (II), compositions thereof, methods and uses thereof described herein can reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the small molecule aryl hydrocarbon receptor antagonists of Formula (I) or Formula (II), compositions thereof, methods and uses thereof described herein may prevent growth and/or kill existing cancer cells, it can be cytostatic and/or cytotoxic. For cancer therapy, efficacy in vivo can, for example, be measured by assessing the duration of survival, duration of progression free survival (PFS), the response rates (RR), duration of response, and/or quality of life.

One aspect of this application provides compounds that are useful for the treatment of diseases, disorders, and conditions characterized by excessive or abnormal cell proliferation. Such diseases include, but are not limited to, a proliferative or hyperproliferative disease, and a neurodegenerative disease. Examples of proliferative and hyperproliferative diseases include, without limitation, cancer. The term “cancer” includes, but is not limited to, the following cancers: breast; ovary; cervix; prostate; testis, genitourinary tract; esophagus; larynx, glioblastoma; neuroblastoma; stomach; skin, keratoacanthoma; lung, epidermoid carcinoma, large cell carcinoma, small cell carcinoma, lung adenocarcinoma; bone; colon; colorectal; adenoma; pancreas, adenocarcinoma; thyroid, follicular carcinoma, undifferentiated carcinoma, papillary carcinoma; seminoma; melanoma; sarcoma; bladder carcinoma; liver carcinoma and biliary passages; kidney carcinoma; myeloid disorders; lymphoid disorders, Hodgkin's, hairy cells; buccal cavity and pharynx (oral), lip, tongue, mouth, pharynx; small intestine; colonrectum, large intestine, rectum, brain and central nervous system; chronic myeloid leukemia (CML), and leukemia. The term “cancer” includes, but is not limited to, the following cancers: myeloma, lymphoma, or a cancer selected from gastric, renal, or and the following cancers: head and neck, oropharangeal, non-small cell lung cancer (NSCLC), endometrial, hepatocarcinoma, Non-Hodgkins lymphoma, and pulmonary.

The term “cancer” refers to any cancer caused by the proliferation of malignant neoplastic cells, such as tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodisplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non-small cell), breast cancer, pancreatic cancer, melanoma and other skin cancers, stomach cancer, brain tumors, tumors related to Gorlin's syndrome (e.g., medulloblastoma, meningioma, etc.), and liver cancer. Additional exemplary forms of cancer which may be treated by the subject compounds include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers that the compounds described herein may be useful in preventing, treating and studying are, for example, colon carcinoma, familiary adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma. In one aspect of the application, the present application provides for the use of one or more compounds of the application in the manufacture of a medicament for the treatment of cancer, including without limitation the various types of cancer disclosed herein.

In some embodiments, the compounds of this application are useful for treating cancer, such as colorectal, thyroid, breast, and lung cancer; and myeloproliferative disorders, such as polycythemia vera, thrombocythemia, myeloid metaplasia with myelofibrosis, chronic myelogenous leukemia, chronic myelomonocytic leukemia, hypereosinophilic syndrome, juvenile myelomonocytic leukemia, and systemic mast cell disease. In some embodiments, the compounds of this application are useful for treating hematopoietic disorders, in particular, acute-myelogenous leukemia (AML), chronic-myelogenous leukemia (CML), acute-promyelocytic leukemia, and acute lymphocytic leukemia (ALL).

This application further embraces the treatment or prevention of cell proliferative disorders such as hyperplasias, dysplasias and pre-cancerous lesions. Dysplasia is the earliest form of pre-cancerous lesion recognizable in a biopsy by a pathologist. The subject compounds may be administered for the purpose of preventing said hyperplasias, dysplasias or pre-cancerous lesions from continuing to expand or from becoming cancerous. Examples of pre-cancerous lesions may occur in skin, esophageal tissue, breast and cervical intra-epithelial tissue.

In accordance with the foregoing, the present application further provides a method for preventing or treating any of the diseases or disorders described above in a subject in need of such treatment, which method comprises administering to said subject a therapeutically effective amount of a compound or aryl hydrocarbon receptor antagonist of the application, or a pharmaceutically acceptable salt, hydrate, or solvate thereof, and optionally a second agent or anti-cancer therapy. For any of the above uses, the required dosage will vary depending on the mode of administration, the particular condition to be treated and the effect desired.

In other embodiments, the compound and the one or more additional anti-cancer therapies are administered simultaneously or sequentially.

Additionally or alternatively, it is equally envisaged that compounds described herein and compositions thereof as aryl hydrocarbon receptor modulators can be used to treat an immunodeficiency, such as a congenital immunodeficiency. Additionally or alternatively, the compositions and methods described herein can be used to treat an acquired immunodeficiency (e.g., an acquired immunodeficiency selected from the group consisting of HIV and AIDS). In these cases, for example, the compounds or compositions thereof may be administered to a patient.

Additionally or alternatively, it is equally envisaged that compounds described herein and compositions thereof as aryl hydrocarbon receptor modulators can be used to treat malignancy or proliferative disorder, such as a hematologic cancer or myeloproliferative disease. In these cases, for example, the compounds or compositions thereof may be administered to a patient. Exemplary hematological cancers that can be treated by way of administration of aryl hydrocarbon receptor modulators in accordance with the compositions and methods described herein are acute myeloid leukemia, acute lymphoid leukemia, chronic myeloid leukemia, chronic lymphoid leukemia, multiple myeloma, diffuse large B-cell lymphoma, and non-Hodgkin's lymphoma, as well as other cancerous conditions, including neuroblastoma.

Additional diseases that can be treated by the administration of aryl hydrocarbon receptor antagonists to a patient include, without limitation, adenosine deaminase deficiency and severe combined immunodeficiency, hyper immunoglobulin M syndrome, Chediak-Higashi disease, hereditary lymphohistiocytosis, osteopetrosis, osteogenesis imperfecta, storage diseases, thalassemia major, systemic sclerosis, systemic lupus erythematosus, multiple sclerosis, and juvenile rheumatoid arthritis.

Additionally or alternatively, it is equally envisaged that compounds described herein and compositions thereof as aryl hydrocarbon receptor modulators can be used to treat automimmune disorders. Autoimmune diseases that can be treated by way of administering aryl hydrocarbon receptor antagonists to a patient include, without limitation, psoriasis, psoriatic arthritis, Type 1 diabetes mellitus (Type 1 diabetes), rheumatoid arthritis (RA), human systemic lupus (SLE), multiple sclerosis (MS), inflammatory bowel disease (IBD), lymphocytic colitis, acute disseminated encephalomyelitis (ADEM), Addison's disease, alopecia universalis, ankylosing spondylitisis, antiphospholipid antibody syndrome (APS), aplastic anemia, autoimmune hemolytic anemia, autoimmune hepatitis, autoimmune inner ear disease (AIED), autoimmune lymphoproliferative syndrome (ALPS), autoimmune oophoritis, Balo disease, Behcet's disease, bullous pemphigoid, cardiomyopathy, Chagas' disease, chronic fatigue immune dysfunction syndrome (CFIDS), chronic inflammatory demyelinating polyneuropathy, Crohn's disease, cicatrical pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome (GBS), Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis, Kawasaki's disease, lichen planus, Lyme disease, Meniere disease, mixed connective tissue disease (MCTD), myasthenia gravis, neuromyotonia, opsoclonus myoclonus syndrome (OMS), optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjögren's syndrome, stiff person syndrome, Takayasu's arteritis, temporal arteritis (also known as “giant cell arteritis”), ulcerative colitis, collagenous colitis, uveitis, vasculitis, vitiligo, vulvodynia (“vulvar vestibulitis”), and Wegener's granulomatosis.

Additionally or alternatively, it is equally envisaged that compounds described herein and compositions thereof as aryl hydrocarbon receptor modulators can be used to treat neurological disorders, such as Parkinson's disease, Alzheimer's disease, multiple sclerosis, Amyotrophic lateral sclerosis, Huntington's disease, mild cognitive impairment, amyloidosis, AIDS-related dementia, encephalitis, stroke, head trauma, epilepsy, mood disorders, and dementia.

The methods disclosed herein for treating disorders in a subject in need thereof comprise the administration of an aryl hydrocarbon receptor antagonist to a subject in need thereof. In one embodiment, the amount of aryl hydrocarbon receptor antagonist administered to the subject is equal to or greater than the amount of aryl hydrocarbon receptor antagonist needed to achieve a therapeutic benefit. In one embodiment, the amount of expanded aryl hydrocarbon receptor antagonist administered to the subject is greater than the amount of aryl hydrocarbon receptor antagonist needed to achieve a therapeutic benefit. In one embodiment, the therapeutic benefit achieved is proportional to the amount of aryl hydrocarbon receptor antagonist that is administered.

A dose of the aryl hydrocarbon receptor antagonist or compositions thereof of the disclosure is deemed to have achieved a therapeutic benefit if it alleviates a sign or a symptom of the disease. The sign or symptom of the disease may comprise one or more biomarkers associated with the disease, or one or more clinical symptoms of the disease.

For example, administration of the aryl hydrocarbon receptor antagonist and compositions thereof may result in the reduction of a biomarker that is elevated in individuals suffering from the disease, or elevate the level of a biomarker that is reduced in individuals suffering from the disease.

For example, administering the aryl hydrocarbon receptor antagonist and compositions thereof of the disclosure may elevate the level of an enzyme that is reduced in an individual suffering from a metabolic disorder. This change in biomarker level may be partial, or the level of the biomarker may return to levels normally seen in healthy individuals.

### Pharmaceutical Compositions and Kits

In some aspects, the disclosure provides a pharmaceutical composition comprising a compound (e.g., aryl hydrocarbon receptor antagonist) described herein and a pharmaceutically acceptable carrier.

In some embodiments, the pharmaceutical composition further comprises one or more additional cancer therapies.

In some aspects, the disclosure provides a kit comprising a compound (e.g., aryl hydrocarbon receptor antagonist) described herein and a pharmaceutically acceptable carrier.

In some embodiments, the kit further comprises one or more additional cancer therapies.

In some embodiments, the kit further comprises a package insert.

Compounds or aryl hydrocarbon receptor antagonists of the application can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present application in free form or in a pharmaceutically acceptable salt form with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present application with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

The pharmaceutical compositions of the present application comprise a therapeutically effective amount of a compound of the present application formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. The pharmaceutical compositions of this application can be administered to humans and other animals orally, rectally, parenterally, intracistemally, intravaginally, intraperitoneally, topically (as by powders, ointments, or drops), buccally, or as an oral or nasal spray.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this application with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid compositions of a similar type may also be employed as fillers in soft and hard filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents.

Dosage forms for topical or transdermal administration of a compound of this application include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this application.

The ointments, pastes, creams and gels may contain, in addition to an active compound of this application, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this application, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to the methods of treatment of the present application, disorders are treated or prevented in a subject, such as a human or other animal, by administering to the subject a therapeutically effective amount of a compound of the application, in such amounts and for such time as is necessary to achieve the desired result. The term “therapeutically effective amount” of a compound of the application, as used herein, means a sufficient amount of the compound so as to decrease the symptoms of a disorder in a subject. As is well understood in the medical arts a therapeutically effective amount of a compound of this application will be at a reasonable benefit/risk ratio applicable to any medical treatment.

In general, compounds of the application will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g., humans, is in the range from about 0.5 mg to about 100 mg, conveniently administered, e.g., in divided doses up to four times a day or in retard form. Suitable unit dosage forms for oral administration comprise from ca. 1 to 50 mg active ingredient.

In certain embodiments, a therapeutic amount or dose of the compounds of the present application may range from about 0.1 mg/Kg to about 500 mg/Kg, alternatively from about 1 to about 50 mg/Kg. In general, treatment regimens according to the present application comprise administration to a patient in need of such treatment from about 10 mg to about 1000 mg of the compound(s) of this application per day in single or multiple doses. Therapeutic amounts or doses will also vary depending on route of administration, as well as the possibility of co-usage with other agents.

Upon improvement of a subject's condition, a maintenance dose of a compound, composition or combination of this application may be administered, if necessary. Subsequently, the dosage or frequency of administration, or both, may be reduced, as a function of the symptoms, to a level at which the improved condition is retained when the symptoms have been alleviated to the desired level, treatment should cease. The subject may, however, require intermittent treatment on a long-term basis upon any recurrence of disease symptoms.

It will be understood, however, that the total daily usage of the compounds and compositions of the present application will be decided by the attending physician within the scope of sound medical judgment. The specific inhibitory dose for any particular patient will depend upon a variety of factors including the disorder being treated and the severity of the disorder, the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The application also provides for a pharmaceutical combinations, e.g., a kit, comprising a) a first agent which is a compound of the application as disclosed herein, in free form or in pharmaceutically acceptable salt form, and optionally b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g., a compound of the application and a co-agent, are both administered to a patient simultaneously in the form of a single entity or dosage. The term “non-fixed combination” means that the active ingredients, e.g., a compound of the application and a co-agent, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

In certain embodiments, these compositions optionally further comprise one or more additional therapeutic agents. For example, an agent that modulates aryl hydrocarbon receptor activity, chemotherapeutic agents or other antiproliferative agents may be combined with the compounds or aryl hydrocarbon receptor antagonists of the present disclosure to treat proliferative diseases and cancers.

Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylenepolyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes, oils such as peanut oil, cottonseed oil; safflower oil; sesame oil; olive oil; corn oil and soybean oil; glycols; such a propylene glycol or polyethylene glycol; esters such as ethyl oleate and ethyl laurate, agar; buffering agents such as magnesium hydroxide and aluminum hydroxide; alginic acid; pyrogen-free water, isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The protein kinase modulators or pharmaceutical salts thereof may be formulated into pharmaceutical compositions for administration to animals or humans. These pharmaceutical compositions, which comprise an amount of the protein modulator effective to treat or prevent a protein kinase-mediated condition and a pharmaceutically acceptable carrier, are other embodiments of the present application.

The application is further illustrated by the following examples and synthesis schemes, which are not to be construed as limiting this application in scope or spirit to the specific procedures herein described. It is to be understood that the examples are provided to illustrate certain embodiments and that no limitation to the scope of the application is intended thereby. It is to be further understood that resort may be had to various other embodiments, modifications, and equivalents thereof which may suggest themselves to those skilled in the art without departing from the spirit of the present application and/or scope of the appended claims.

### EXAMPLES

The following examples are put forth so as to provide those of ordinary skill in the art with a description of how the compositions and methods described herein may be used, made, and evaluated, and are intended to be purely exemplary and are not intended to limit the scope of what the inventors regard as their invention.

### Example 1. Synthesis of Compound (5)

Compound (5) can be synthesized, for example, using a Hartwig-Buchwald amination process that includes coupling a halogenated imidazopyridine precursor to a protected 2-aminoethyl indole, as shown in Scheme 8, below.

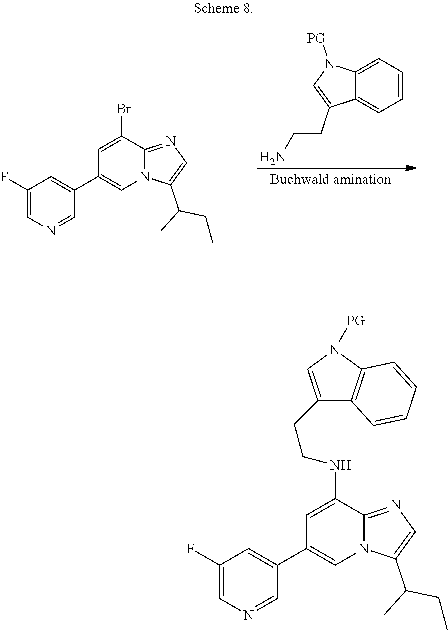

Deprotection of the ensuing adduct, for instance, using conventional deprotection methods known in the art, can yield compound (5).

Alternatively, the tandem amination-hydrolysis procedure outline in Scheme 9, below, may be used to synthesize compound (5).

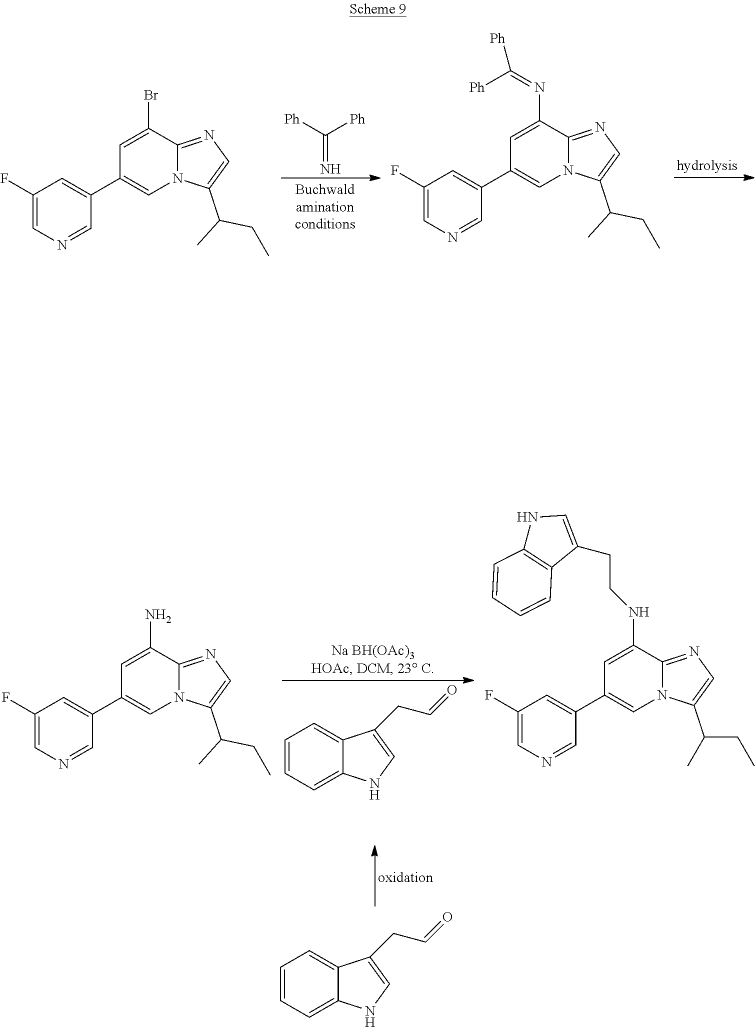

### Example 2. Synthesis of Compound (16)

In a manner similar to that described in Example 1, compound (16) can be synthesized by way of a Hartwig-Buchwald process, followed by a Suzuki coupling and indole deprotection, as shown in Scheme 10, below.

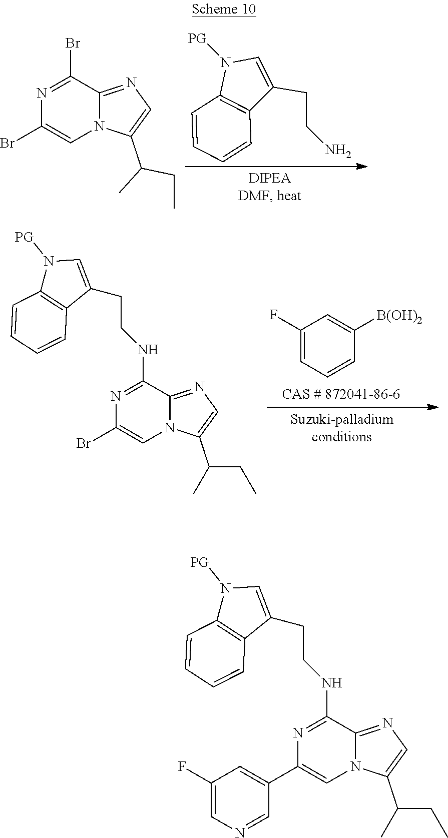

### Example 3. Capacity of Compounds (5) and (16) to Expand Hematopoietic Stem Cells

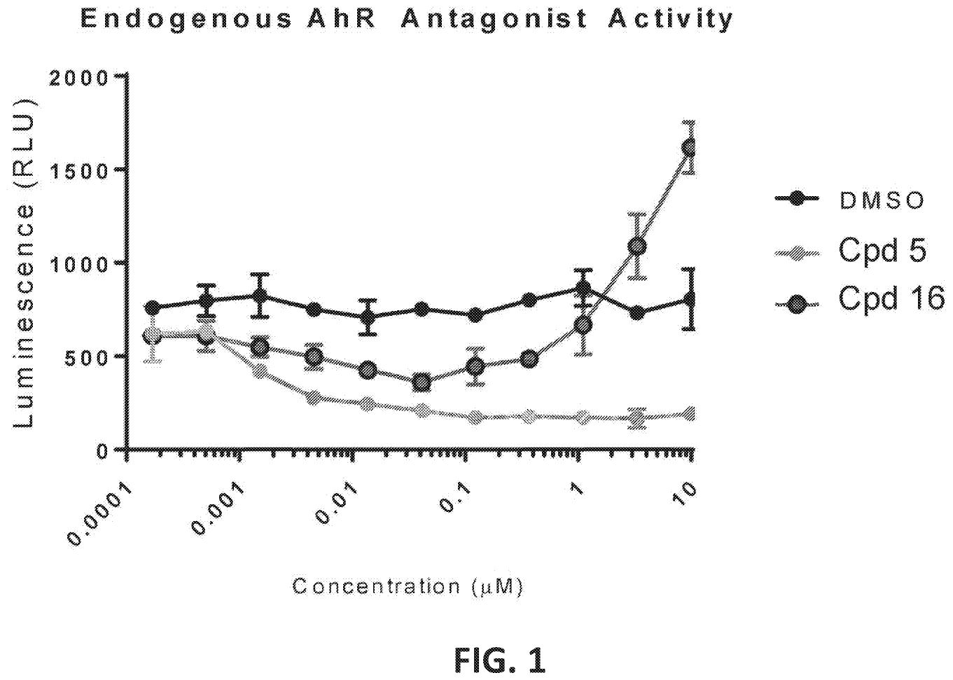

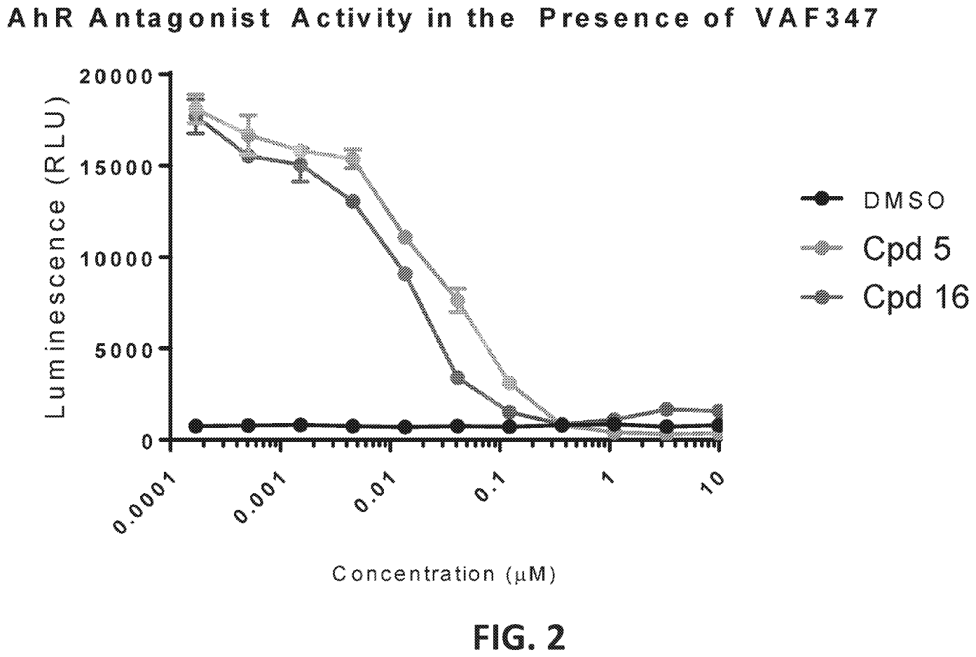

To determine the ability of compounds (5) and (16) to inhibit the activity of the aryl hydrocarbon receptor and to induce the proliferation of hematopoietic stem cells, a series of HSC expansion experiments were conducted. In the first experiment, compounds (5) and (16) were assessed for their capacity to attenuate aryl hydrocarbon receptor signaling. To this end, HepG2 hepatocytes were transiently transfected with a luciferase reporter construct under the control of a promoter responsive to aryl hydrocarbon receptor signal transduction. The cells were plated at a density of 25,000 cells per well in a microtiter plate. The HepG2 cells were immediately treated with compound (5) or (16) in the absence (FIG. 1) or presence (FIG. 2) of the aryl hydrocarbon receptor agonist, VAF347 (80 nM). Luciferase activity was subsequently analyzed six hours after plating.

As shown in FIGS. 1 and 2, compounds (5) and (16) were capable of suppressing aryl hydrocarbon receptor activity even in the presence of the activator VAF347.

### Example 4. Synthesis of Compound (24)

In a manner similar to that described in Example 1 and Example 2, compound (24) can be synthesized by way of an arene iodination and Hartwig-Buchwald process, followed by a first Suzuki coupling, a second Suzuki coupling and alkene reduction by catalytic hydrogenation over palladium as shown in Scheme 11, below.

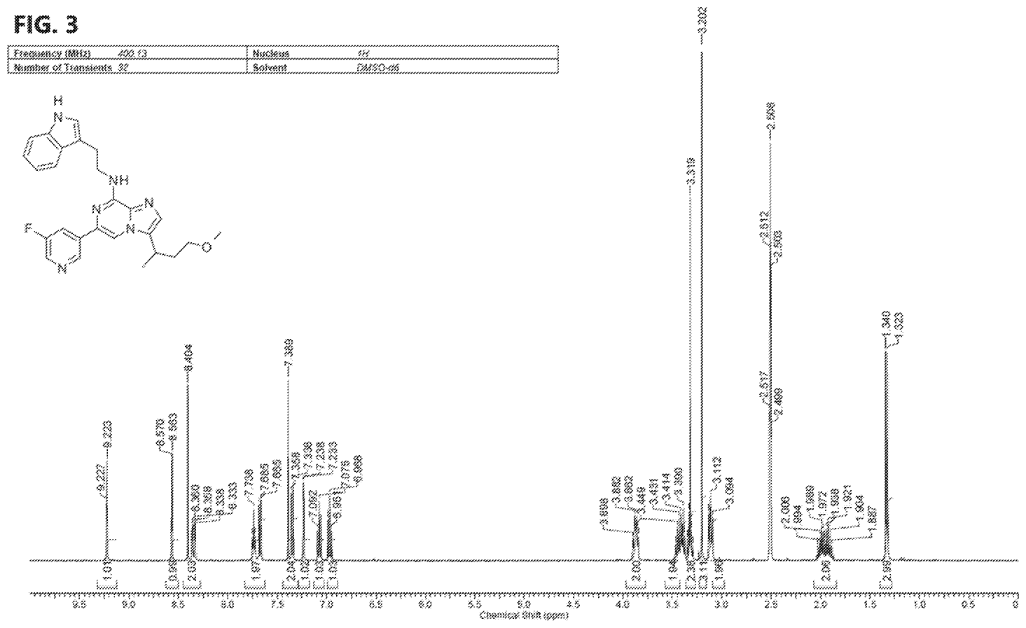

6-(5-fluoropyridin-3-yl)-N-[2-(1H-indol-3-yl)ethyl]-3-(3-methoxy-1-methylpropyl)imidazo[1,2-a]pyrazin-8-amine, Compound (24) was isolated as a white solid. FIG. 3 sets forth a ¹H-NMR spectrum (d6-DMSO) consistent with the structure. HPLC analysis gave 97.93% Area at 254 nm and 97.65% Area at 210 nm; Retention time: 3.765 min; HPLC conditions: Agilent 1100 HPLC. Zorbax Eclipse XDBC18 50×4.6 mm 1.8 micron column. Solvent A: Water (0.1% TFA); Solvent B: Acetonitrile (0.07% TFA). Gradient: 95% A to 95% B over 5 min; hold for 1 min; recycle over 1 min; 30 s hold. UV Detection: 210 and 254 nm with no reference. Column temperature: 30° C. Mass spectrum was consistent with structure MS (ESI+) for C₂₆H₂₇FN₆O m/z 459.1 (M+H)+; MS (ESI−) for C₂₆H₂₇FN₆O m/z 457.2 (M−H)−.

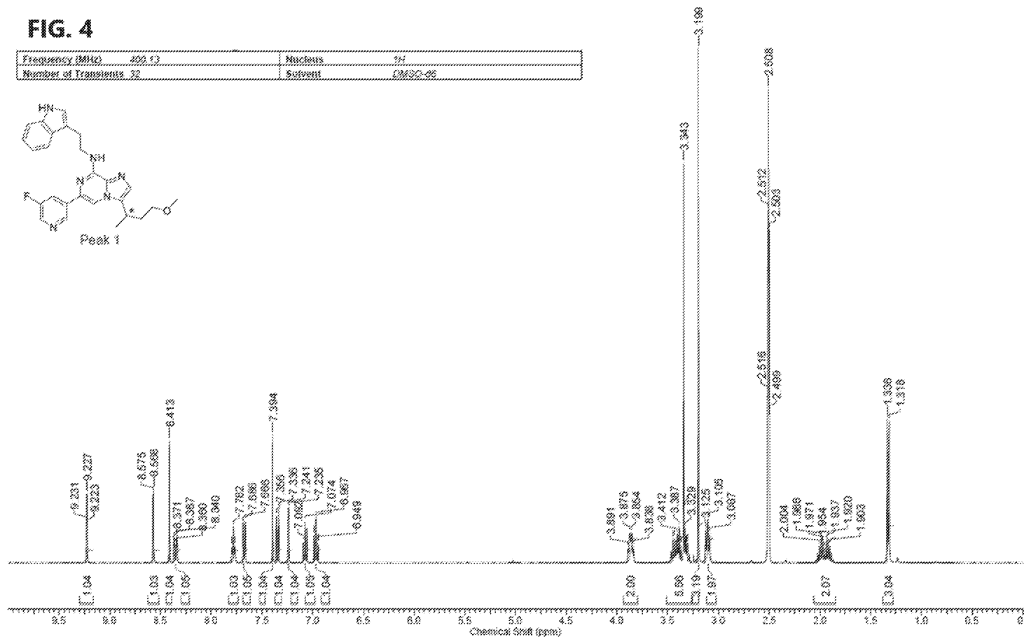

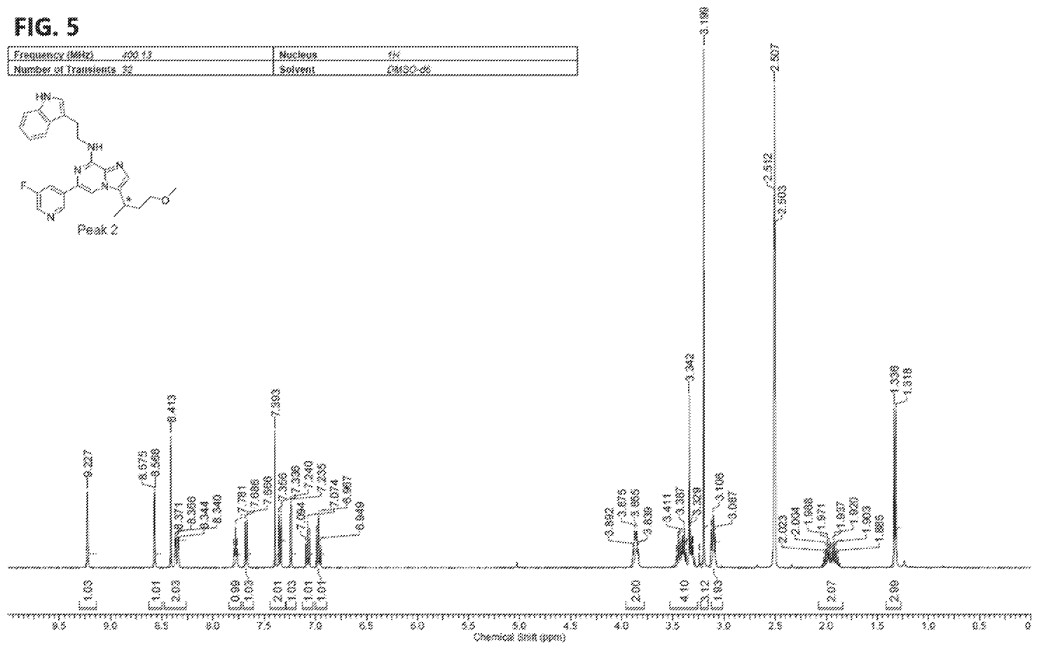

Preparative HPLC AD-H (2×25 cm) 25% methanol/CO₂, 100 bar, 65 mL/min, 220 nm, inj vol.: 0.5 mL, 20 mg/mL methanol:DCM and analytical HPLC AD-H (25×0.46 cm) 35% methanol/CO₂, 100 bar 3 mL/min, 220, 254 and 280 nm allowed for the resolution of distinct enatiomeric peaks for Compound (24). FIG. 4 sets forth a ¹H-NMR spectrum (d6-DMSO) consistent with the structure isolated as peak 1. FIG. 5 sets forth a H-NMR spectrum (d6-DMSO) consistent with the structure isolated as peak 2.

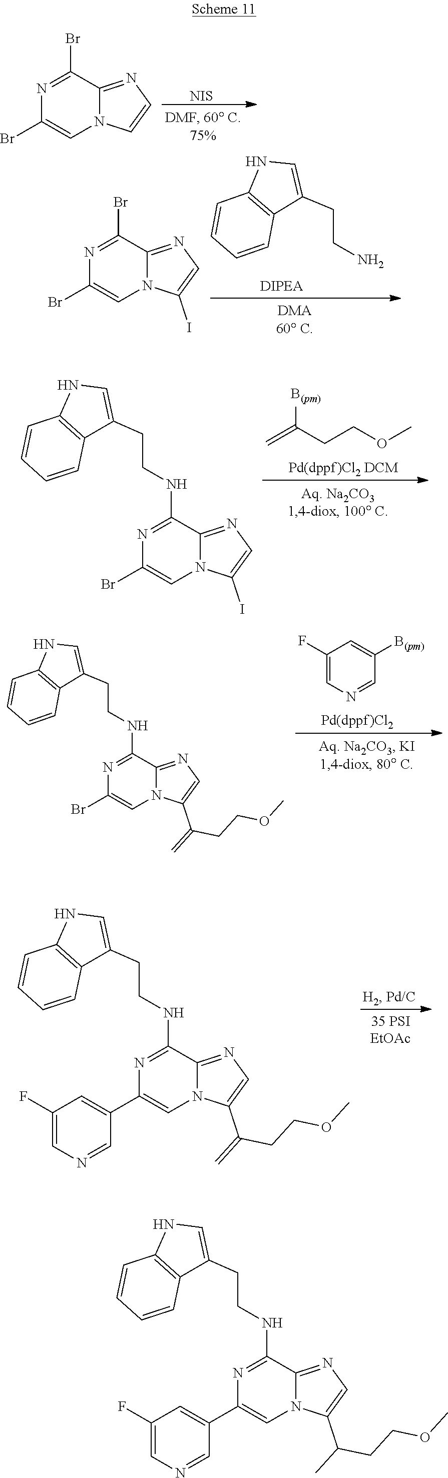

### Example 5. Synthesis of Compound (27)

In a manner similar to that described in Example 4, compound (27) can be synthesized by way of a first Suzuki coupling, and second Suzuki coupling and alkene reduction by catalytic hydrogenation over palladium as shown in Scheme 12, below.

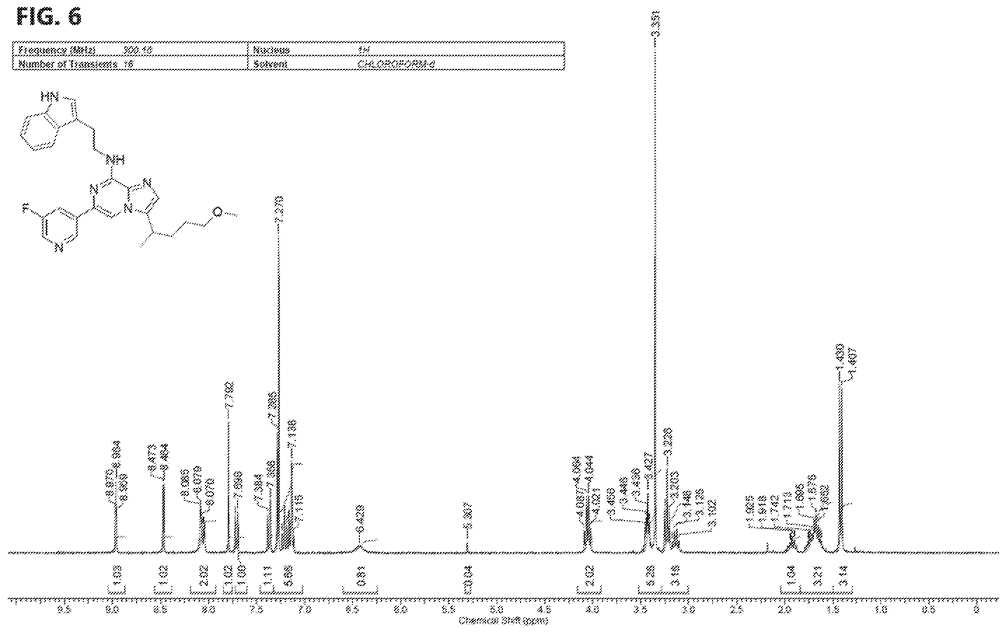

6-(5-fluoropyridin-3-yl)-N-[2-(1H-indol-3-yl)ethyl]-3-(4-methoxy-1-methylbutyl)imidazo[1,2-a]pyrazin-8-amine, Compound (27) was isolated as a white solid. FIG. 6 sets forth a ¹H-NMR spectrum (CDCl₆) consistent with the structure. HPLC analysis gave 98.0% Area at 254 nm and 97.2% Area at 210 nm; Retention time: 3.670 min; HPLC conditions: Agilent 1100 HPLC. Zorbax Eclipse XDBC18; 50×4.6 mm 1.8 micron column. Solvent A: Water (0.1% TFA); Solvent B: Acetonitrile (0.07% TFA). Gradient: 95% A to 95% B over 5 min; hold for 1 min; recycle over 1 min; 30 s hold. UV Detection: 210 and 254 nm with no reference. Column temperature: 30° C. Mass Spectrum was consistent with structure MS (ESI+) for C₂₇H₂₉FN₆O m/z 473.1 (M+H)+; MS (ESI−) for C₂₇H₂₉FN₆O m/z 471.2 (M−H)−

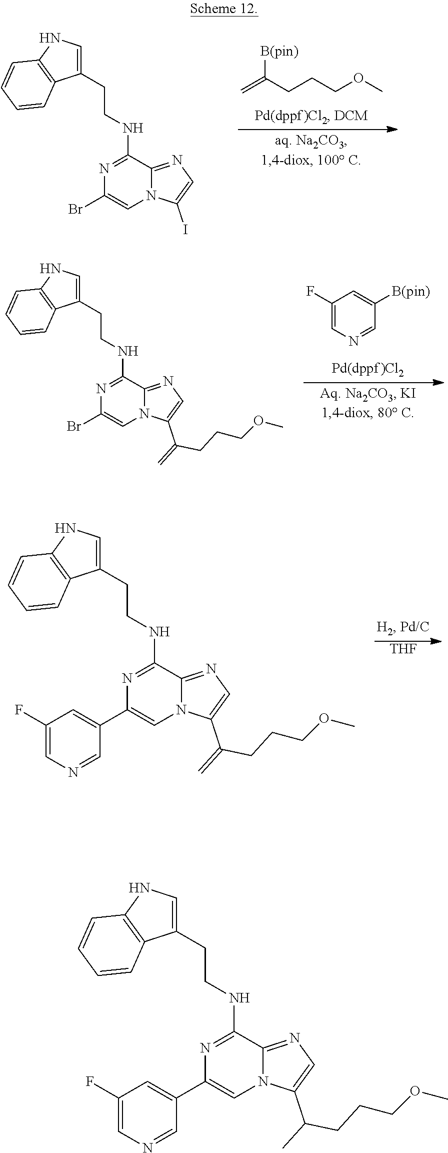

### Example 6. Synthesis of Compound (28)

In a manner similar to that described in Example 4 and Example 5, compound (28) can be synthesized by way of a first Suzuki coupling, and second Suzuki coupling and alkene reduction by catalytic hydrogenation over palladium as shown in Scheme 13, below.

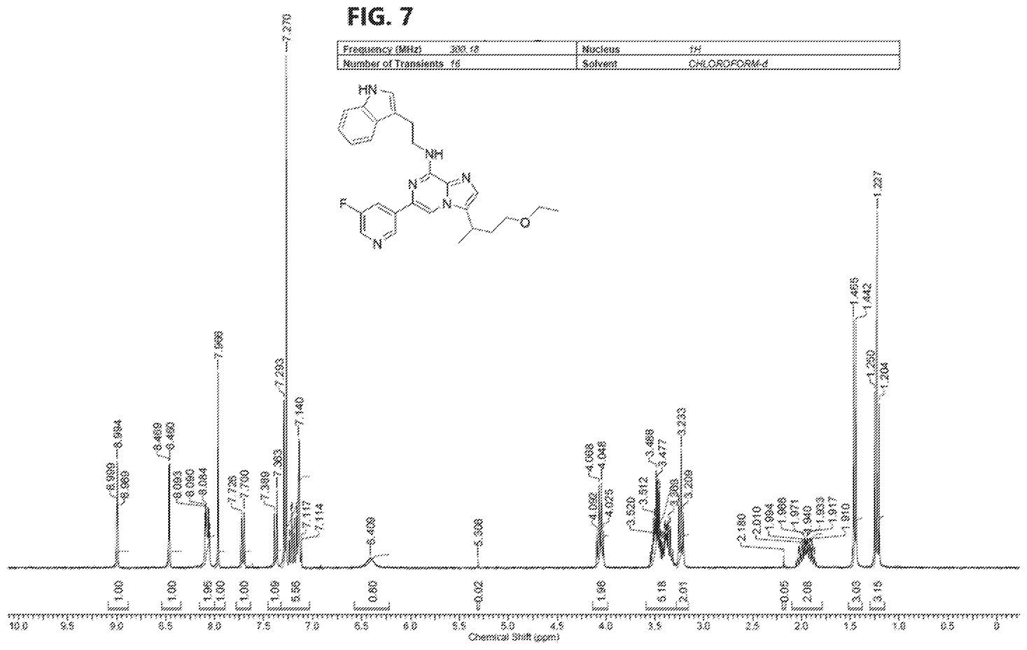

3-(3-ethoxy-1-methylpropyl)-6-(5-fluoropyridin-3-yl)-N-[2-(1H-indol-3-yl)ethyl]imidazo[1,2-a]pyrazin-8-amine, Compound (28) was isolated as an off-white solid. FIG. 7 sets forth a ¹H-NMR spectrum (CDCl₃) consistent with the structure. HPLC analysis gave 99.3% Area at 254 nm and 99.0% Area at 210 nm; Retention time: 3.757 min; HPLC conditions: Agilent 1100 HPLC. Zorbax Eclipse XDBC18; 50×4.6 mm 1.8 micron column. Solvent A: Water (0.1% TFA); Solvent B: Acetonitrile (0.07% TFA). Gradient: 95% A to 95% B over 5 min; hold for 1 min; recycle over 1 min; 30 s hold. UV Detection: 210 and 254 nm with no reference. Column temperature: 30° C. Mass spectrum was consistent with structure MS (ESI+) for C₂₆H₂FN₆O m/z 473.1 (M+H)+; MS (ESI−) for C₇H₂FN₆O m/z 471.2 (M−H)−.

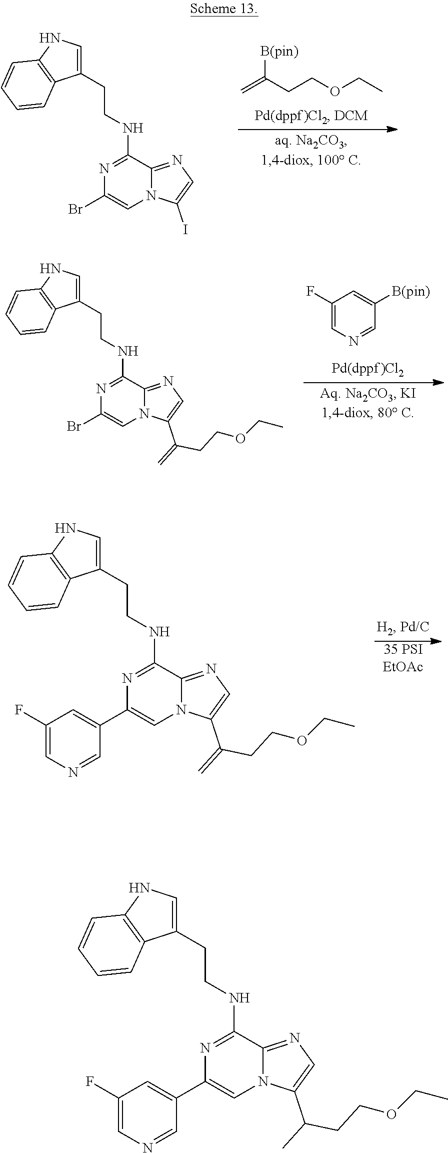

Suitable substrates may be provided for the first Suzuki coupling in the synthesis of compound (24), compound (27), and compound (28) in Example 4, Example 5, and Example 6, respectively, by alkylation of a suitable alkynyl alcohol and borylation of the alkyne as shown in Scheme 14, below.

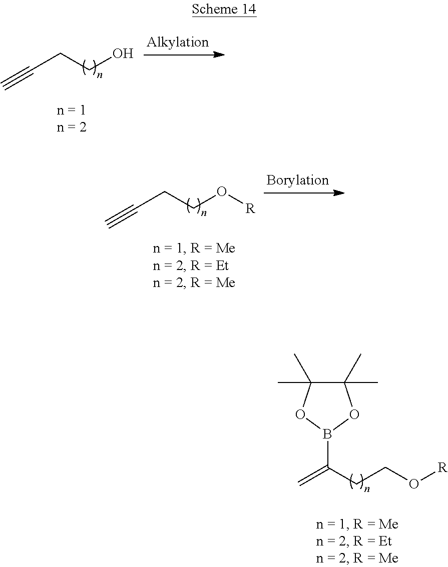

### Example 7. Evaluation of AHR Antagonists

To determine the activity of a scale-up batch of Compound (24), individual enantiomers, and two derivatives Compound (27) and Compound (28) were evaluated for AHR antagonist activity. The compounds were compared to control AHR antagonist SR1, an additional control AHR antagonist, and stored sample of Compound (24)

### AHR Antagonist Assay

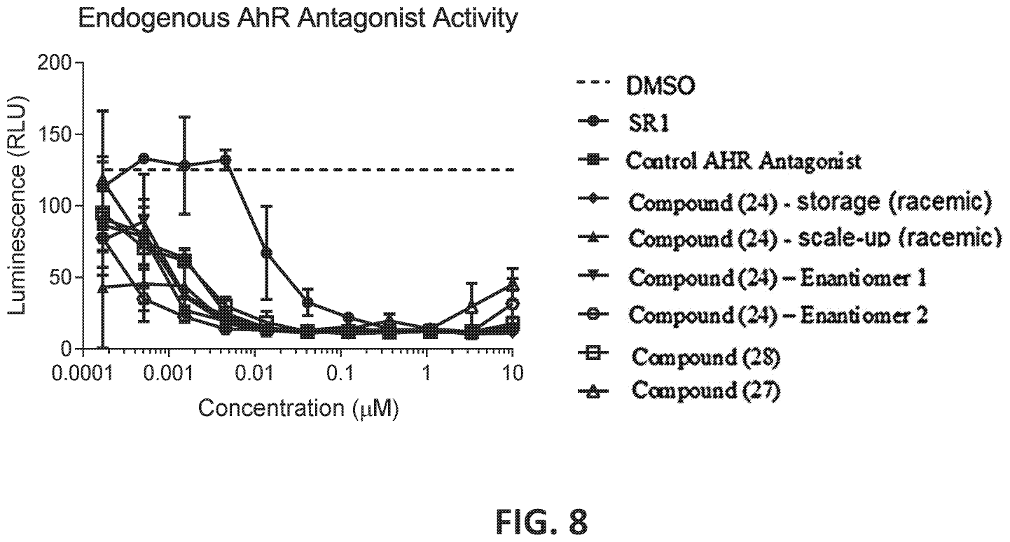

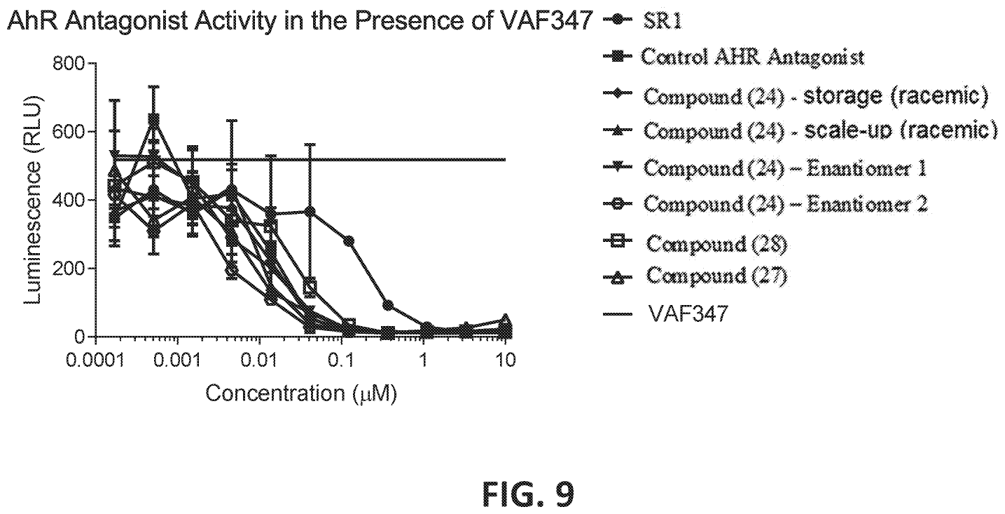

Methods—HepG2 cells transiently transfected with the pGudLuc8.1 plasmid were thawed and 25,000 cells were plated per well and immediately treated the AHR agonist VAF347 (fixed at 40 nM) and/or the indicated AHR antagonists. Luciferase activity was measured 24 hours post-culture, corresponding to endogenous AHR antagonist activity (without VAF347, FIG. 8) or in the presence of VAF347 (FIG. 9). All synthesized compounds show equivalent AHR antagonist activity if not greater than SR1.

TABLE 1
Endogenous AHR Antagonist Activity

| Compound | EC50 (μM) |
|---|---|
| SR1 | 0.033 |
| Control AHR Antagonist | 0.0009 |
| Compound (24) - storage (racemic) | 0.001 |
| Compound (24) - scale-up (racemic) | ND |
| Compound (24) - Enantiomer Peak 1 | 0.001 |
| Compound (24) - Enantiomer Peak 2 | 0.0004 |
| Compound (28) | 0.002 |
| Compound (27) | 0.002 |

TABLE 2
AHR Antagonist Activity in the Presence of VAF347

| Compound | EC50 (μM) |
|---|---|
| SR1 | 0.193 |
| Control AHR Antagonist | 0.014 |
| Compound (24) - storage (racemic) | 0.013 |
| Compound (24) - scale-up (racemic) | 0.011 |
| Compound (24) - Enantiomer Peak 1 | 0.012 |
| Compound (24) - Enantiomer Peak 2 | 0.006 |
| Compound (28) | 0.030 |
| Compound (27) | 0.009 |

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of modulating the activity of an aryl hydrocarbon receptor, comprising administering to a subject in need thereof an effective amount of an aryl hydrocarbon receptor antagonist.

2. A method of treating or preventing a disease or disorder, comprising administering to a subject in need thereof an effective amount of an aryl hydrocarbon receptor antagonist.

3. An aryl hydrocarbon receptor antagonist for use in modulating the activity of an aryl hydrocarbon receptor in a subject in need thereof.

4. An aryl hydrocarbon receptor antagonist for use in treating or preventing a disease or disorder in a subject in need thereof.

5. Use of an aryl hydrocarbon receptor antagonist in the preparation or manufacture of a medicament for modulating the activity of an aryl hydrocarbon receptor in a subject in need thereof.

6. Use of an aryl hydrocarbon receptor antagonist in the preparation or manufacture of a medicament for treating or preventing a disease or disorder in a subject in need thereof.

7. A pharmaceutical composition comprising an aryl hydrocarbon receptor antagonist and a pharmaceutically acceptable carrier.

8. A kit comprising an aryl hydrocarbon receptor antagonist and a pharmaceutically acceptable carrier.

9. The pharmaceutical composition or kit of any one of the preceding claims for use in modulating the activity of an aryl hydrocarbon receptor in a subject in need thereof.

10. The pharmaceutical composition or kit of any one of the preceding claims for use in treating or preventing a disease or disorder in a subject in need thereof.

11. The method, use, aryl hydrocarbon receptor antagonist, pharmaceutical composition, or kit of any one of the preceding claims, wherein the disease or disorder is characterized by the production of an aryl hydrocarbon receptor agonist.

12. The method, use, aryl hydrocarbon receptor antagonist, pharmaceutical composition, or kit of any one of the preceding claims, wherein the disease or disorder is a cancer, a cancerous condition, or a tumor.

13. The method, use, aryl hydrocarbon receptor antagonist, pharmaceutical composition, or kit of any one of the preceding claims, wherein the tumor is a solid tumor.

14. The method, use, aryl hydrocarbon receptor antagonist, pharmaceutical composition, or kit of any one of the preceding claims, wherein the tumor is an invasive tumor.

15. The method, use, aryl hydrocarbon receptor antagonist, pharmaceutical composition, or kit of any one of the preceding claims, wherein the cancer is a breast cancer, squamous cell cancer, lung cancer, a cancer of the peritoneum, a hepatocellular cancer, a gastric cancer, a pancreatic cancer, a glioblastoma, a cervical cancer, an ovarian cancer, a liver cancer, a bladder cancer, a hepatoma, a colon cancer, a colorectal cancer, an endometrial or uterine carcinoma, a salivary gland carcinoma, a kidney or renal cancer, a prostate cancer, a vulval cancer, a thyroid cancer, a head and neck cancer, a B-cell lymphoma, a chronic lymphocytic leukemia (CLL), an acute lymphoblastic leukemia (ALL), a Hairy cell leukemia, or a chronic myeloblastic leukemia.

16. The method, use, aryl hydrocarbon receptor antagonist, pharmaceutical composition, or kit of any one of the preceding claims, wherein one or more additional anti-cancer therapies is administered to the subject.

17. The method, use, aryl hydrocarbon receptor antagonist, pharmaceutical composition, or kit of any one of the preceding claims, wherein the aryl hydrocarbon receptor antagonist is a compound represented by formula (I) 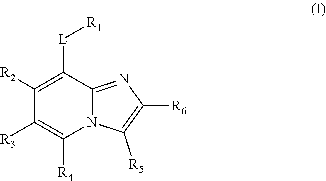
or a pharmaceutically acceptable salt, hydrate, or solvate thereof,
wherein L is selected from the group consisting of —NR₇ₐ(CR₈ₐR_{8b})ₙ—, —O(CR₈ₐR_{8b})ₙ—, —C(O)(CR₈ₐR_{8b})ₙ—, —C(S)(CR₈ₐR_{8b})ₙ—, —S(O)₀₋₂(CR₈ₐR_{8b})ₙ—, —(CR₈ₐR_{8b})ₙ—, —NR₇ₐC(O)(CR₈ₐR_{8b})ₙ—, —NR₇ₐC(S)(CR₈ₐR_{8b})ₙ—, —OC(O)(CR₈ₐR_{8b})ₙ—, —OC(S)(CR₈ₐR_{8b})ₙ—, —C(O)NR₇ₐ(CR₉ₐR₉ₐ)ₙ—, —C(S)NR₇ₐ(CR₈ₐR_{8b})ₙ—, —C(O)O(CR₈ₐR_{8b})ₙ—, —C(S)O(CR₈ₐR_{8b})ₙ—, —S(O)₂NR₇ₐ(CR₉ₐR_{9b})ₙ—, —NR₇ₐS(O)₂(CR₈ₐR_{8b})ₙ—, —NR₇ₐC(O)NR_{7b}(CR₉ₐR₉ₐ)ₙ—, and —NR₇ₐC(O)O(CR₈ₐR_{8b})ₙ—, wherein R₇ₐ, R_{7b}, R₈ₐ, and R_{8b} are each independently selected from the group consisting of hydrogen and optionally substituted C₁-4 alkyl, and each n is independently an integer from 2 to 6;
R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉ₐ, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)CR₉ₐR_{9b}R_{9c}, —OC(S)CR_{9b}R_{9c}, optionally substituted aryl, optionally substituted heteroaryl, optionally substituted cycloalkyl, and optionally substituted heterocycloalkyl, wherein R₉ₐ, R_{9b}, and R_{9c} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;
R₂ is selected from the group consisting of hydrogen and optionally substituted C₁-4 alkyl;
R₃ is selected from the group consisting of optionally substituted aryl, optionally substituted heteroaryl, optionally substituted cycloalkyl, and optionally substituted heterocycloalkyl;
R₄ is selected from the group consisting of hydrogen and optionally substituted C₁-4 alkyl;
R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and
R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

18. The method, use, aryl hydrocarbon receptor antagonist, pharmaceutical composition, or kit of any one of the preceding claims, wherein R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)CR₉ₐR_{9b}R_{9c}, —OC(S)CR₉ₐR_{9b}R_{9c}, phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein said phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C₁-4 alkyl, C₁-4 alkoxy, halo, halo-substituted-C₁-4 alkyl, halo-substituted-C₁-4 alkoxy, amino, —O(CH₂)₂NR₁₀ₐR_{10b}, —S(O)₂NR₁₀ₐR_{10b}, —OS(O)₂NR₁₀ₐR_{10b} and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

19. The method of any one of the preceding claims, wherein R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)CR₉ₐR_{9b}R_{9c}, and —OC(S)CR₉ₐR_{9b}R_{9c}.

20. The method, use, aryl hydrocarbon receptor antagonist, pharmaceutical composition, or kit of any one of the preceding claims, wherein R₁ is selected from the group consisting of phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein said phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}.

21. The method, use, aryl hydrocarbon receptor antagonist, pharmaceutical composition, or kit of any one of the preceding claims, wherein R₁ is selected from the group consisting of phenyl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, and 2-oxo-2,3-dihydro-1H-benzo[d]imidazol-5-yl, wherein said phenyl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, or 2-oxo-2,3-dihydro-1H-benzo[d]imidazol-5-yl is optionally substituted with from 1 to 3 substituents independently selected from the group consisting of cyano, hydroxy, C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}.

22. The method, use, aryl hydrocarbon receptor antagonist, pharmaceutical composition, or kit of any one of the preceding claims, wherein R₁ is selected from the group consisting of phenyl, phenol-4-yl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, and 2-oxo-2,3-dihydro-1H-benzo[d]imidazol-5-yl.

23. The method, use, aryl hydrocarbon receptor antagonist, pharmaceutical composition, or kit of any one of the preceding claims, wherein R₁ is selected from the group consisting of phenol-4-yl and 1H-indol-3-yl.

24. The method, use, aryl hydrocarbon receptor antagonist, pharmaceutical composition, or kit of any one of the preceding claims, wherein L is selected from the group consisting of —NR₇ₐ(CR₈ₐR_{8b}b)ₙ- and —O(CR₈ₐR_{8b})ₙ—.

25. The method, use, aryl hydrocarbon receptor antagonist, pharmaceutical composition, or kit of any one of the preceding claims, wherein R₂ is hydrogen.

26. The method, use, aryl hydrocarbon receptor antagonist, pharmaceutical composition, or kit of any one of the preceding claims, wherein R₃ is selected from the group consisting of phenyl, thiophenyl, furanyl, 1H-benzoimidazolyl, quinolinyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl, wherein said phenyl, thiophenyl, furanyl, 1H-benzoimidazolyl, quinolinyl, isoquinolinyl, imidazopyridinyl, benzothiophenyl, pyrimidinyl, pyridinyl, 1H-imidazolyl, pyrazinyl, pyridazinyl, 1H-pyrrolyl, or thiazolyl is optionally substituted with from 1 to 3 substituents independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}, and wherein R₁₁ₐ and R_{11b} are each independently selected from the group consisting of hydrogen and C₁₋₄alkyl.

27. The method, use, aryl hydrocarbon receptor antagonist, pharmaceutical composition, or kit of any one of the preceding claims, wherein R₃ is selected from the group consisting of thiophen-2-yl, thiophen-3-yl, furan-3-yl, 1H-benzo[d]imidazol-1-yl, isoquinolin-4-yl, 1H-imidazo[4,5-b]pyridin-1-yl, imidazo[1,2-a]pyridin-3-yl, benzo[b]thiophen-3-yl, pyrimidin-5-yl, pyridin-2-yl, pyridin-3-yl, pyridin-4-yl, 1H-imidazol-1-yl, pyrazin-2-yl, pyridazin-4-yl, 1H-pyrrol-2-yl and thiazol-5-yl, wherein said thiophen-2-yl, thiophen-3-yl, furan-3-yl, 1H-benzo[d]imidazol-1-yl, isoquinolin-4-yl, 1H-imidazo[4,5-b]pyridin-1-yl, benzo[b]thiophen-3-yl, pyrimidin-5-yl, pyridin-2-yl, pyridin-3-yl, pyridin-4-yl, 1H-imidazol-1-yl, pyrazin-2-yl, pyridazin-4-yl, 1H-pyrrol-2-yl, or thiazol-5-yl is optionally substituted with from 1 to 3 substituents independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}.

28. The method, use, aryl hydrocarbon receptor antagonist, pharmaceutical composition, or kit of any one of the preceding claims, wherein R₃ is selected from the group consisting of thiophen-3-yl, benzo[b]thiophen-3-yl, pyridin-3-yl, pyrimidin-5-yl, 1H-imidazol-1-yl, 1H-benzo[d]imidazol-1-yl, isoquinolin-4-yl, 1H-imidazo[4,5-b]pyridin-1-yl, and imidazo[1,2-a]pyridin-3-yl, wherein said thiophen-3-yl, benzo[b]thiophen-3-yl, pyridin-3-yl, pyrimidin-5-yl, 1H-imidazol-1-yl, 1H-benzo[d]imidazol-1-yl, isoquinolin-4-yl, 1H-imidazo[4,5-b]pyridin-1-yl, or imidazo[1,2-a]pyridin-3-yl is optionally substituted with from 1 to 3 substituents independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}.

29. The method, use, aryl hydrocarbon receptor antagonist, pharmaceutical composition, or kit of any one of the preceding claims, wherein R₃ is pyridin-3-yl, wherein said pyridin-3-yl is optionally substituted at C5 with a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

30. The method, use, aryl hydrocarbon receptor antagonist, pharmaceutical composition, or kit of any one of the preceding claims, wherein said pyridin-3-yl is substituted at C5 with a substituent selected from the group consisting of ethoxycarbonyl, methoxy, cyano, methyl, methylsulfonyl, fluoro, chloro, trifluoromethyl, ethynyl, and cyclopropyl.

31. The method, use, aryl hydrocarbon receptor antagonist, pharmaceutical composition, or kit of any one of the preceding claims, wherein R₃ is imidazo[1,2-a]pyridin-3-yl, wherein said imidazo[1,2-a]pyridin-3-yl is optionally substituted with a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

32. The method, use, aryl hydrocarbon receptor antagonist, pharmaceutical composition, or kit of any one of the preceding claims, wherein R₃ is benzo[b]thiophen-3-yl, wherein said benzo[b]thiophen-3-yl is optionally substituted with a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

33. The method, use, aryl hydrocarbon receptor antagonist, pharmaceutical composition, or kit of any one of the preceding claims, wherein R₃ is 1H-imidazo[4,5-b]pyridin-1-yl, wherein said 1H-imidazo[4,5-b]pyridin-1-yl is optionally substituted with a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

34. The method, use, aryl hydrocarbon receptor antagonist, pharmaceutical composition, or kit of any one of the preceding claims, wherein R₃ is isoquinolin-4-yl, wherein said isoquinolin-4-yl is optionally substituted with a substituent selected from the group consisting of C1-4 alkyl, halo, halo-substituted-C1-4 alkyl, C1-4 alkenyl, C1-4 alkynyl, C2-8 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

35. The method, use, aryl hydrocarbon receptor antagonist, pharmaceutical composition, or kit of any one of the preceding claims, wherein R₄ is hydrogen.

36. The method, use, aryl hydrocarbon receptor antagonist, pharmaceutical composition, or kit of any one of the preceding claims, wherein R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein said C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4alkyl.

37. The method, use, aryl hydrocarbon receptor antagonist, pharmaceutical composition, or kit of any one of the preceding claims, wherein R₃ is selected from the group consisting of isopropyl, methyl, ethyl, prop-1-en-2-yl, isobutyl, cyclohexyl, sec-butyl, (S)-sec-butyl, (R)-sec-butyl, 1-hydroxypropan-2-yl, (S)-1-hydroxypropan-2-yl, (R)-1-hydroxypropan-2-yl, and nonan-2-yl.

38. The method, use, aryl hydrocarbon receptor antagonist, pharmaceutical composition, or kit of any one of the preceding claims, wherein R₅ is selected from the group consisting of (i), (ii), (iii), (iv), and (v) 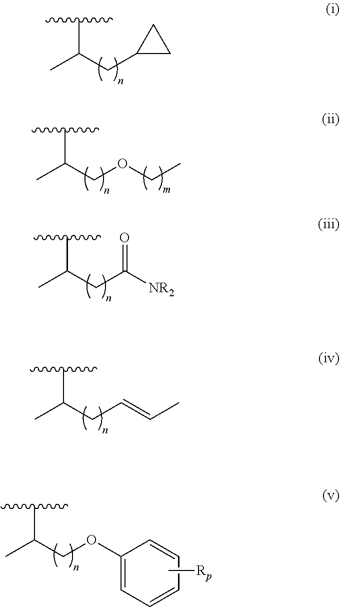
wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

39. The method, use, aryl hydrocarbon receptor antagonist, pharmaceutical composition, or kit of any one of the preceding claims, wherein Re is hydrogen.

40. The method, use, aryl hydrocarbon receptor antagonist, pharmaceutical composition, or kit of any one of the preceding claims, wherein said aryl hydrocarbon receptor antagonist is a compound represented by formula (I-a) 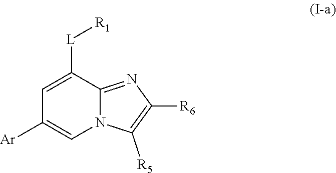
or a pharmaceutically acceptable salt, hydrate, or solvate thereof,
wherein L is a linker selected from the group consisting of —NR₇ₐ(CR₈ₐR_{8b})ₙ—, —O(CR₈ₐR_{8b})ₙ—, —C(O)(CR₈ₐR_{8b})ₙ—, —C(S)(CR₈ₐR_{8b})ₙ—, —S(O)₀₋₂(CR₈ₐR_{8b})ₙ—, —(CR₈ₐR_{8b})ₙ—, —NR₇ₐC(O)(CR₈ₐR_{8b})ₙ—, —NR₇ₐC(S)(CR₈ₐR_{8b})ₙ—, —OC(O)(CR₈ₐR_{8b})ₙ—, —OC(S)(CR₈ₐR_{8b})ₙ—, —C(O)NR₇ₐ(CR₈ₐR_{8b})ₙ—, —C(S)NR₇ₐ(CR₈ₐR_{8b})ₙ—, —C(O)O(CR₈ₐR_{8b})ₙ—, —C(S)O(CR₈ₐR_{8b})ₙ—, —S(O)₂NR₇ₐ(CR₈ₐR_{8b})ₙ—, —NR₇ₐS(O)₂(CR₈ₐR_{8b})ₙ—, —NR₇ₐC(O)NR_{7b}(CR₈ₐR_{8b})ₙ—, and —NR₇ₐC(O)O(CR₈ₐR_{8b})ₙ—, wherein R₇ₐ, R_{7b}, R₈ₐ, and R_{8b} are each independently selected from the group consisting of hydrogen and optionally substituted C1-4 alkyl, and each n is independently an integer from 2 to 6;
R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉ₐ, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)CR₉ₐR_{9b}R_{9c}, —OC(S)CR₉ₐR_{9b}R_{9c}, optionally substituted aryl, optionally substituted heteroaryl, optionally substituted cycloalkyl, and optionally substituted heterocycloalkyl, wherein R₉ₐ, R_{9b}, and R_{9c} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;
Ar is selected from the group consisting of optionally substituted monocyclic aryl and heteroaryl;
R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and
R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

41. The method, use, aryl hydrocarbon receptor antagonist, pharmaceutical composition, or kit of any one of the preceding claims, wherein R₁ is selected from the group consisting of phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

42. The method, use, aryl hydrocarbon receptor antagonist, pharmaceutical composition, or kit of any one of the preceding claims, wherein said aryl hydrocarbon receptor is a compound represented by formula (I-b) 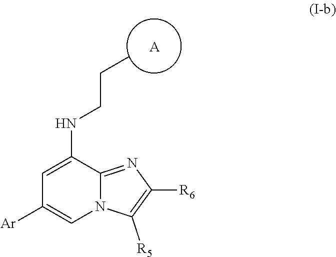
or a pharmaceutically acceptable salt, hydrate, or solvate thereof,
wherein A is an optionally substituted ring system selected from the group consisting of phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C1-4 alkyl, C1-alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;
Ar is selected from the group consisting of optionally substituted monocyclic aryl and heteroaryl;
R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and
R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

43. The method, use, aryl hydrocarbon receptor antagonist, pharmaceutical composition, or kit of any one of the preceding claims, wherein said aryl hydrocarbon receptor antagonist is a compound represented by formula (I-c) 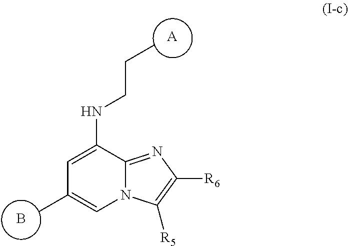
or a pharmaceutically acceptable salt, hydrate, or solvate thereof,
wherein A is an optionally substituted ring system selected from the group consisting of phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C1-4 alkyl, C₁₋₄ alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;
B is an optionally substituted ring system selected from the group consisting of thiophenyl, furanyl, 1H-benzoimidazolyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl, wherein the thiophenyl, furanyl, 1H-benzoimidazolyl, isoquinolinyl, 1H-imidazopyridinyl, benzothiophenyl, pyrimidinyl, pyridinyl, 1H-imidazolyl, pyrazinyl, pyridazinyl, 1H-pyrrolyl, or thiazolyl is optionally substituted with from 1 to 3 substituents independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl;
R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and
R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

44. The method, use, aryl hydrocarbon receptor antagonist, pharmaceutical composition, or kit of any one of the preceding claims, wherein said aryl hydrocarbon receptor antagonist is a compound represented by formula (I-d) 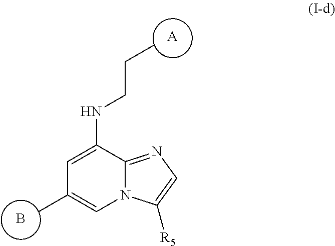
or a pharmaceutically acceptable salt, hydrate, or solvate thereof,
wherein A is an optionally substituted ring system selected from the group consisting of phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C1-4alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b} and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;
B is an optionally substituted ring system selected from the group consisting of thiophenyl, furanyl, 1H-benzoimidazolyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl, wherein the thiophenyl, furanyl, 1H-benzoimidazolyl, isoquinolinyl, 1H-imidazopyridinyl, benzothiophenyl, pyrimidinyl, pyridinyl, 1H-imidazolyl, pyrazinyl, pyridazinyl, 1H-pyrrolyl, or thiazolyl is optionally substituted with from 1 to 3 substituents independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and
R₃ is selected from the group consisting of optionally substituted aryl, optionally substituted heteroaryl, optionally substituted alkyl, optionally substituted heteroalkyl, optionally substituted cycloalkyl, and optionally substituted heterocycloalkyl.

45. The method of any one of the preceding claims, wherein said aryl hydrocarbon receptor antagonist is a compound represented by formula (I-e) 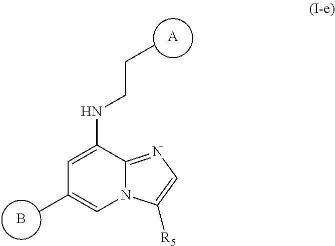
or a pharmaceutically acceptable salt, hydrate, or solvate thereof,
wherein A is an optionally substituted ring system selected from the group consisting of phenyl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, and 2-oxo-2,3-dihydro-1H-benzo[d]imidazol-5-yl, wherein the phenyl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, or 2-oxo-2,3-dihydro-1H-benzo[d]imidazol-5-yl is optionally substituted with from 1 to 3 substituents independently selected from the group consisting of cyano, hydroxy, C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;
B is an optionally substituted ring system selected from the group consisting of thiophen-2-yl, thiophen-3-yl, furan-3-yl, 1H-benzo[d]imidazol-1-yl, isoquinolin-4-yl, 1H-imidazo[4,5-b]pyridin-1-yl, imidazo[1,2-a]pyridin-3-yl, benzo[b]thiophen-3-yl, pyrimidin-5-yl, pyridin-2-yl, pyridin-3-yl, pyridin-4-yl, 1H-imidazol-1-yl, pyrazin-2-yl, pyridazin-4-yl, 1H-pyrrol-2-yl and thiazol-5-yl, wherein the thiophen-2-yl, thiophen-3-yl, furan-3-yl, 1H-benzo[d]imidazol-1-yl, isoquinolin-4-yl, 1H-imidazo[4,5-b]pyridin-1-yl, benzo[b]thiophen-3-yl, pyrimidin-5-yl, pyridin-2-yl, pyridin-3-yl, pyridin-4-yl, 1H-imidazol-1-yl, pyrazin-2-yl, pyridazin-4-yl, 1H-pyrrol-2-yl, or thiazol-5-yl is optionally substituted with from 1 to 3 substituents independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and
R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4alkyl, or R₅ is selected from the group consisting of (i), (ii), (iii), (iv), and (v) 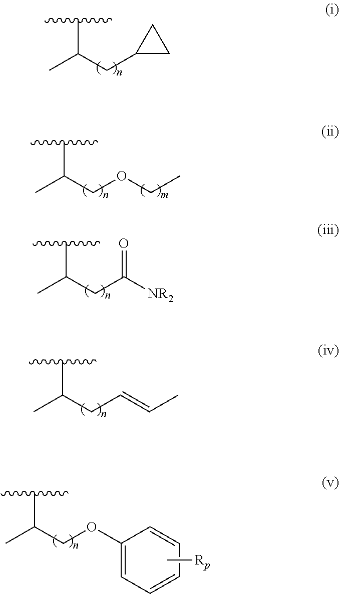
wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

46. The method, use, aryl hydrocarbon receptor antagonist, pharmaceutical composition, or kit of any one of the preceding claims, wherein said aryl hydrocarbon receptor antagonist is a compound represented by formula (I-f) 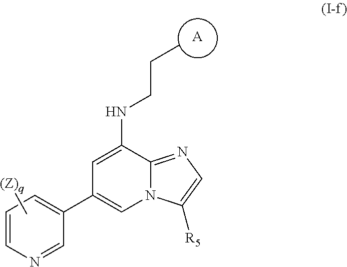
or a pharmaceutically acceptable salt, hydrate, or solvate thereof,
wherein A is an optionally substituted ring system selected from the group consisting of phenol-4-yl and 1H-indol-3-yl;
q is an integer from 0 to 4;
each Z is independently a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₉ₐR_{11b}, wherein R₁₁ₐ and R_{11b} are each independently selected from the group consisting of hydrogen and C₁₋₄ alkyl; and
R₅ is selected from the group consisting of isopropyl, methyl, ethyl, prop-1-en-2-yl, isobutyl, cyclohexyl, sec-butyl, (S)-sec-butyl, (R)-sec-butyl, 1-hydroxypropan-2-yl, (S)-1-hydroxypropan-2-yl, (R)-1-hydroxypropan-2-yl, and nonan-2-yl, or R₅ is selected from the group consisting of (i), (ii), (iii), (iv), and (v) 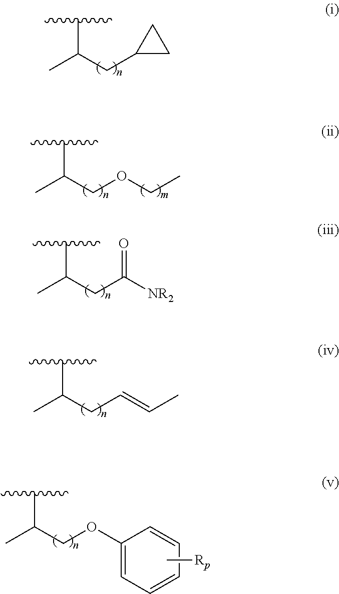
wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

47. The method, use, aryl hydrocarbon receptor antagonist, pharmaceutical composition, or kit of any one of the preceding claims, wherein said aryl hydrocarbon receptor antagonist is a compound represented by formula (I-g) 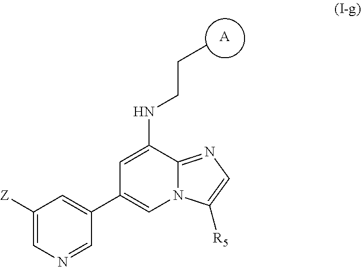
or a pharmaceutically acceptable salt, hydrate, or solvate thereof,
wherein A is an optionally substituted ring system selected from the group consisting of phenol-4-yl and 1H-indol-3-yl;
Z is a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)N₁₁ₐR_{11b}, wherein R₁₁ₐ and Rum are each independently selected from the group consisting of hydrogen and C₁₋₄ alkyl; and
R₃ is selected from the group consisting of isopropyl, methyl, ethyl, prop-1-en-2-yl, isobutyl, cyclohexyl, sec-butyl, (S)-sec-butyl, (R)-sec-butyl, 1-hydroxypropan-2-yl, (S)-1-hydroxypropan-2-yl, (R)-1-hydroxypropan-2-yl, and nonan-2-yl, or R₅ is selected from the group consisting of (i), (ii), (iii), (iv), and (v) 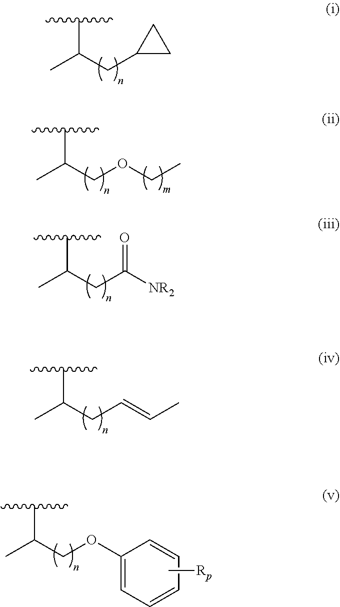
wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12c}, and wherein R₁₂ₐ and R_{12b} are each independently selected from the group consisting of hydrogen and C₁₋₄alkyl.

48. The method, use, aryl hydrocarbon receptor antagonist, pharmaceutical composition, or kit of any one of the preceding claims, wherein said aryl hydrocarbon receptor antagonist is a compound represented by formula (I-h) 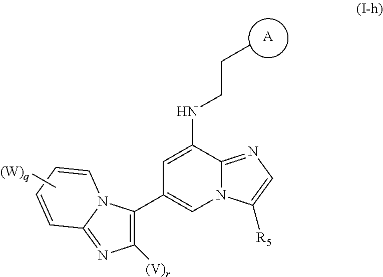
or a pharmaceutically acceptable salt, hydrate, or solvate thereof,
wherein A is an optionally substituted ring system selected from the group consisting of phenol-4-yl and 1H-indol-3-yl;
q is an integer from 0 to 4;
r is 0 or 1;
W and V are each independently a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄alkyl; and
R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4alkyl, or R₅ is selected from the group consisting of (i), (ii), (iii), (iv), and (v) 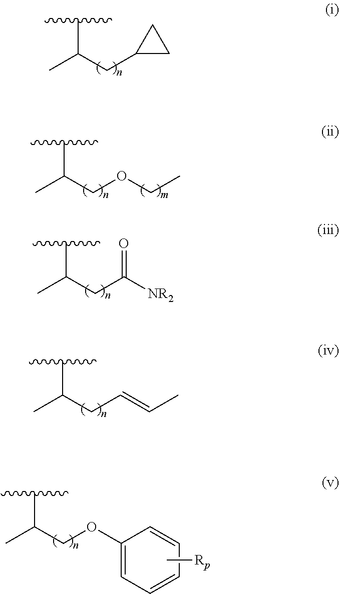
wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

49. The method, use, aryl hydrocarbon receptor antagonist, pharmaceutical composition, or kit of any one of the preceding claims, wherein said aryl hydrocarbon receptor antagonist is a compound represented by formula (I-i) 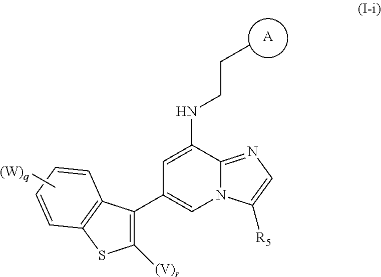
or a pharmaceutically acceptable salt, hydrate, or solvate thereof,
wherein A is an optionally substituted ring system selected from the group consisting of phenol-4-yl and 1H-indol-3-yl;
q is an integer from 0 to 4;
r is 0 or 1;
W and V are each independently a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄alkyl; and
R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4-alkyl, or R₅ is selected from the group consisting of (I), (ii), (iii), (lv), and (v) 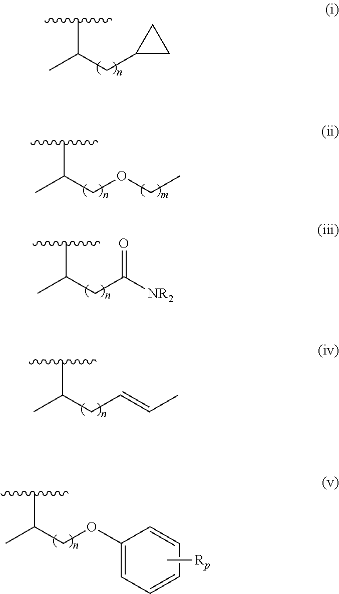
wherein n is an integer from 1 to 6, m is an integer from 0 to 6, p is an integer from 0 to 5, and each R is independently selected from the group consisting of cyano, hydroxy, C1-4 alkyl, C1-4 alkenyl, C14 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

50. The method, use, aryl hydrocarbon receptor antagonist, pharmaceutical composition, or kit of any one of the preceding claims, wherein said aryl hydrocarbon receptor antagonist is a compound represented by formula (I-j) 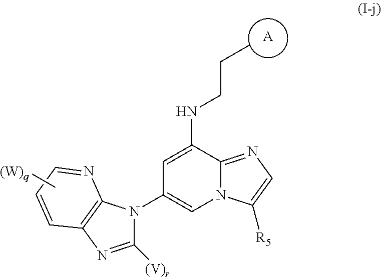
or a pharmaceutically acceptable salt, hydrate, or solvate thereof,
wherein A is an optionally substituted ring system selected from the group consisting of phenol-4-yl and 1H-indol-3-yl;
q is an integer from 0 to 4;
r is 0 or 1;
W and V are each independently a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄alkyl; and
R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4alkyl, or R₅ is selected from the group consisting of (i), (ii), (iii), (iv), and (v) 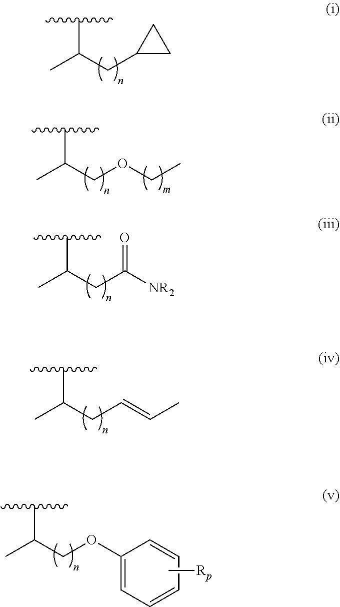
wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄alkyl.

51. The method, use, aryl hydrocarbon receptor antagonist, pharmaceutical composition, or kit of any one of the preceding claims, wherein said aryl hydrocarbon receptor antagonist is a compound represented by formula (I-k) 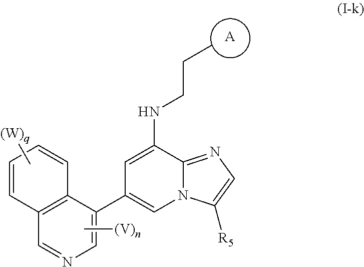
or a pharmaceutically acceptable salt, hydrate, or solvate thereof,
wherein A is an optionally substituted ring system selected from the group consisting of phenol-4-yl and 1H-indol-3-yl;
q is an integer from 0 to 4;
r is 0 or 1;
W and V are each independently a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄alkyl; and
R₃ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4alkyl, or R₅ is selected from the group consisting of (i), (ii), (iii), (iv), and (v) 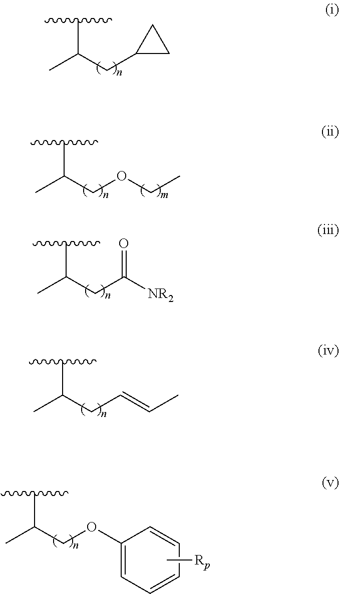
wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

52. The method, use, aryl hydrocarbon receptor antagonist, pharmaceutical composition, or kit of any one of the preceding claims, wherein said aryl hydrocarbon receptor antagonist is compound (1) 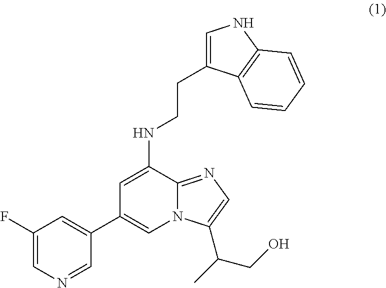 [SMILES: CC(CO)c1cnc2c(NCCc3c[nH]c4ccccc34)cc(-c3cncc(F)c3)cn12]
or a salt thereof.

53. The method, use, aryl hydrocarbon receptor antagonist, pharmaceutical composition, or kit of any one of the preceding claims, wherein said aryl hydrocarbon receptor antagonist is compound (2) 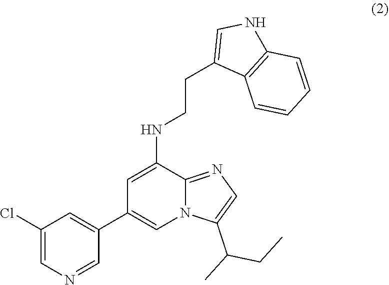 [SMILES: CCC(C)c1cnc2c(NCCc3c[nH]c4ccccc34)cc(-c3cncc(Cl)c3)cn12]
or a salt thereof.

54. The method, use, aryl hydrocarbon receptor antagonist, pharmaceutical composition, or kit of any one of the preceding claims, wherein said aryl hydrocarbon receptor antagonist is compound (3) 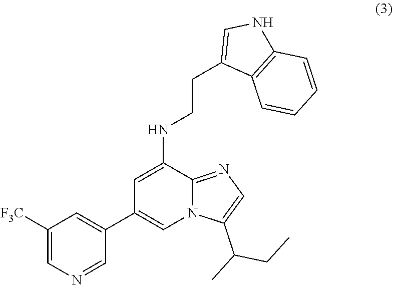 [SMILES: CCC(C)c1cnc2c(NCCc3c[nH]c4ccccc34)cc(-c3cncc(C(F)(F)F)c3)cn12]
or a salt thereof.

55. The method, use, aryl hydrocarbon receptor antagonist, pharmaceutical composition, or kit of any one of the preceding claims, wherein said aryl hydrocarbon receptor antagonist is compound (4) 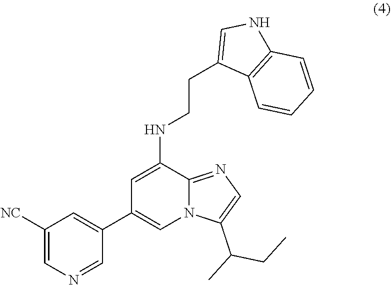 [SMILES: CCC(C)c1cnc2c(NCCc3c[nH]c4ccccc34)cc(-c3cncc(C#N)c3)cn12]
or a salt thereof.

56. The method, use, aryl hydrocarbon receptor antagonist, pharmaceutical composition, or kit of any one of the preceding claims, wherein said aryl hydrocarbon receptor antagonist is compound (5) 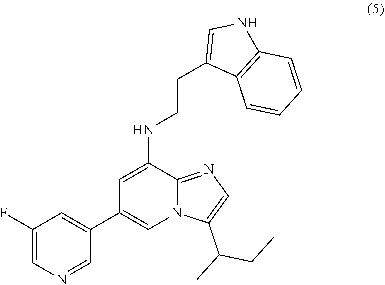 [SMILES: CCC(C)c1cnc2c(NCCc3c[nH]c4ccccc34)cc(-c3cncc(F)c3)cn12]
or a salt thereof.

57. The method, use, aryl hydrocarbon receptor antagonist, pharmaceutical composition, or kit of any one of the preceding claims, wherein said aryl hydrocarbon receptor antagonist is compound (6) 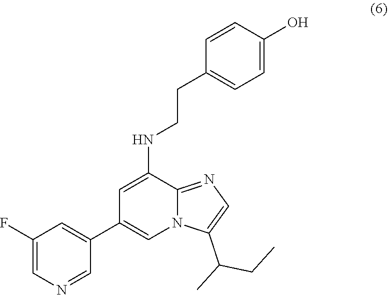 [SMILES: CCC(C)c1cnc2c(NCCc3ccc(O)cc3)cc(-c3cncc(F)c3)cn12]
or a salt thereof.

58. The method, use, aryl hydrocarbon receptor antagonist, pharmaceutical composition, or kit of any one of the preceding claims, wherein said aryl hydrocarbon receptor antagonist is compound (7) 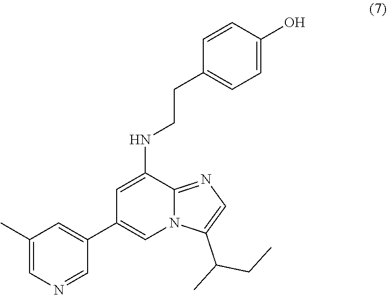 [SMILES: CCC(C)c1cnc2c(NCCc3ccc(O)cc3)cc(-c3cncc(C)c3)cn12]
or a salt thereof.

59. The method, use, aryl hydrocarbon receptor antagonist, pharmaceutical composition, or kit of any one of the preceding claims, wherein said aryl hydrocarbon receptor antagonist is compound (8) 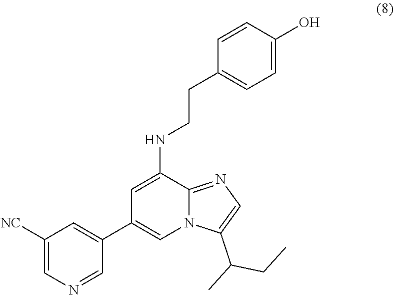 [SMILES: CCC(C)c1cnc2c(NCCc3ccc(O)cc3)cc(-c3cncc(C#N)c3)cn12]
or a salt thereof.

60. The method, use, aryl hydrocarbon receptor antagonist, pharmaceutical composition, or kit of any one of the preceding claims, wherein said aryl hydrocarbon receptor antagonist is compound (9) 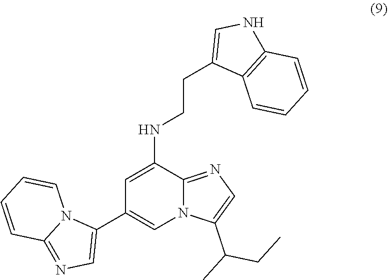 [SMILES: CCC(C)c1cnc2c(NCCc3c[nH]c4ccccc34)cc(-c3cnc4ccccn34)cn12]
or a salt thereof.

61. The method, use, aryl hydrocarbon receptor antagonist, pharmaceutical composition, or kit of any one of the preceding claims, wherein said aryl hydrocarbon receptor antagonist is compound (10) 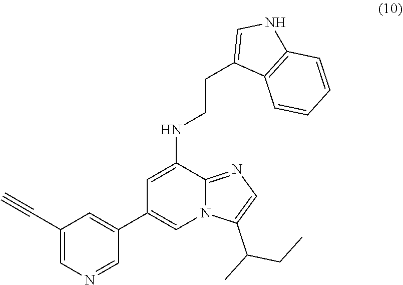 [SMILES: C#Cc1cncc(-c2cc(NCCc3c[nH]c4ccccc34)c3ncc(C(C)CC)n3c2)c1]
or a salt thereof.

62. The method, use, aryl hydrocarbon receptor antagonist, pharmaceutical composition, or kit of any one of the preceding claims, wherein said aryl hydrocarbon receptor antagonist is compound (11) 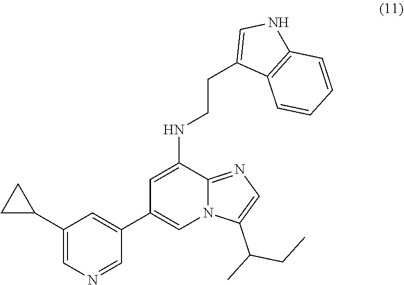 [SMILES: CCC(C)c1cnc2c(NCCc3c[nH]c4ccccc34)cc(-c3cncc(C4CC4)c3)cn12]
or a salt thereof.

63. The method, use, aryl hydrocarbon receptor antagonist, pharmaceutical composition, or kit of any one of the preceding claims, wherein said aryl hydrocarbon receptor antagonist is compound (23) 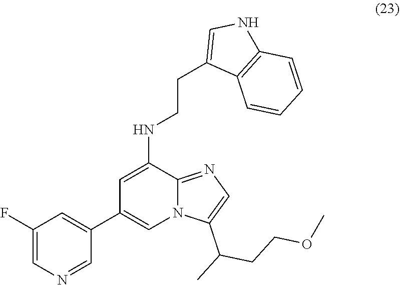 [SMILES: COCCC(C)c1cnc2c(NCCc3c[nH]c4ccccc34)cc(-c3cncc(F)c3)cn12]
or a salt thereof.

64. The method, use, aryl hydrocarbon receptor antagonist, pharmaceutical composition, or kit of any one of the preceding claims, wherein said aryl hydrocarbon receptor antagonist is compound (25) 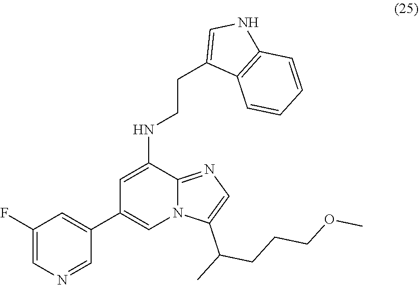 [SMILES: COCCCC(C)c1cnc2c(NCCc3c[nH]c4ccccc34)cc(-c3cncc(F)c3)cn12]
or a salt thereof.

65. The method, use, aryl hydrocarbon receptor antagonist, pharmaceutical composition, or kit of any one of the preceding claims, wherein said aryl hydrocarbon receptor antagonist is compound (26) 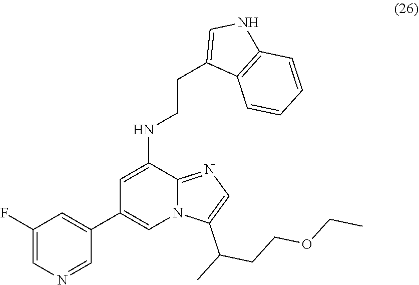 [SMILES: CCOCCC(C)c1cnc2c(NCCc3c[nH]c4ccccc34)cc(-c3cncc(F)c3)cn12]
or a salt thereof.

66. The method, use, aryl hydrocarbon receptor antagonist, pharmaceutical composition, or kit of any one of the preceding claims, wherein the aryl hydrocarbon receptor antagonist is a compound represented by formula (II) 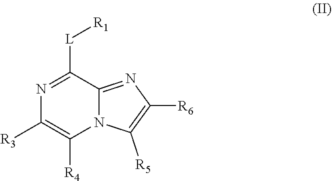
or a pharmaceutically acceptable salt, hydrate, or solvate thereof,
wherein L is selected from the group consisting of —NR₇ₐ(CR₈ₐR_{8b})ₙ—, —O(CR_{9b})ₙ—, —C(O)(CR₈ₐR_{8b})ₙ—, —C(S)(CR₈ₐR_{8b})ₙ—, —S(O)₀₋₂(CR₈ₐR_{8b})ₙ, —(CR₈ₐR_{8b})ₙ—, —NR₇ₐC(O)(CR₈ₐR_{8b})ₙ—, —NR₇ₐC(S)(CR₈ₐR_{8b})ₙ—, —OC(O)(CR₈ₐR_{8b})ₙ—, —OC(S)(CR₈ₐR_{8b})ₙ—, —C(O)NR₇ₐ(CR₈ₐR_{8b})ₙ—, —C(S)NR₇ₐ(CR₈ₐR_{8b})ₙ—, —C(O)O(CR₈ₐR_{8b})ₙ—, —C(S)O(CR₈ₐR_{8b})ₙ—, —S(O)₂NR₇ₐ(CR₈ₐR_{8b})ₙ—, —NR₇ₐS(O)₂(CR₈ₐR_{8b})ₙ—, —NR₇ₐC(O)NR_{7b}(CR₉ₐR₉ₐ)ₙ—, and —NR₇ₐC(O)O(CR₈ₐR_{8b})ₙ—, wherein R₇ₐ, R_{7b}, R₈ₐ, and R₉ₐ are each independently selected from the group consisting of hydrogen and optionally substituted C1-4 alkyl, and each n is independently an integer from 2 to 6;
R₁ is selected from the group consisting of —S(O)₂NR₉ₐR_{9b}, —NR₉ₐC(O)R_{9b}, —NR₉ₐC(S)R_{9b}, —NR₉ₐC(O)NR₉ₐR_{9b}, —C(O)R₉ₐ, —C(S)R₉ₐ, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)NR₉ₐR_{9b}, —OC(S)CR₉ₐR_{9b}R_{9c}, optionally substituted aryl, optionally substituted heteroaryl, optionally substituted cycloalkyl, and optionally substituted heterocycloalkyl, wherein R₉ₐ, R_{9b}, and R_{9c} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;
R₃ is selected from the group consisting of optionally substituted aryl, optionally substituted heteroaryl, optionally substituted cycloalkyl, and optionally substituted heterocycloalkyl;
R₄ is selected from the group consisting of hydrogen and optionally substituted C1-4 alkyl;
R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and
R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

67. The method, use, aryl hydrocarbon receptor antagonist, pharmaceutical composition, or kit of any one of the preceding claims, wherein R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉ₐ, —S(O)₀₋₂R_{9c}, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)CR₉ₐR_{9b}R_{9c}, —OC(S)CR₉ₐR_{9b}R_{9c}, phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein said phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

68. The method, use, aryl hydrocarbon receptor antagonist, pharmaceutical composition, or kit of any one of the preceding claims, wherein R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉ₐ, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)CR₉ₐR_{9b}R_{9c}, and —OC(S)CR₉ₐR_{9b}R_{9c}.

69. The method, use, aryl hydrocarbon receptor antagonist, pharmaceutical composition, or kit of any one of the preceding claims, wherein R₁ is selected from the group consisting of phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein said phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}.

70. The method, use, aryl hydrocarbon receptor antagonist, pharmaceutical composition, or kit of any one of the preceding claims, wherein R₁ is selected from the group consisting of phenyl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, and 2-oxo-2,3-dihydro-1H-benzo[d]imidazol-5-yl, wherein said phenyl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, or 2-oxo-2,3-dihydro-1H-benzo[d]imidazol-5-yl is optionally substituted with from 1 to 3 substituents independently selected from the group consisting of cyano, hydroxy, C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}.

71. The method, use, aryl hydrocarbon receptor antagonist, pharmaceutical composition, or kit of any one of the preceding claims, wherein R₁ is selected from the group consisting of phenyl, phenol-4-yl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, and 2-oxo-2,3-dihydro-1H-benzo[d]imidazol-5-yl.

72. The method, use, aryl hydrocarbon receptor antagonist, pharmaceutical composition, or kit of any one of the preceding claims, wherein R₁ is selected from the group consisting of phenol-4-yl and 1H-indol-3-yl.

73. The method, use, aryl hydrocarbon receptor antagonist, pharmaceutical composition, or kit of any one of the preceding claims, wherein L is selected from the group consisting of —NR₇ₐ(CR₈ₐR_{8b}b)ₙ- and —O(CR₈ₐR_{8b})ₙ—.

74. The method, use, aryl hydrocarbon receptor antagonist, pharmaceutical composition, or kit of any one of the preceding claims, wherein R₃ is selected from the group consisting of phenyl, thiophenyl, furanyl, 1H-benzoimidazolyl, quinolinyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl, wherein said phenyl, thiophenyl, furanyl, 1H-benzoimidazolyl, quinolinyl, isoquinolinyl, imidazopyridinyl, benzothiophenyl, pyrimidinyl, pyridinyl, 1H-imidazolyl, pyrazinyl, pyridazinyl, 1H-pyrrolyl, or thiazolyl is optionally substituted with from 1 to 3 substituents independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}, and wherein R₁₁ₐ and R_{11b} are each independently selected from the group consisting of hydrogen and C₁₋₄alkyl.

75. The method, use, aryl hydrocarbon receptor antagonist, pharmaceutical composition, or kit of any one of the preceding claims, wherein R₃ is selected from the group consisting of thiophen-2-yl, thiophen-3-yl, furan-3-yl, 1H-benzo[d]imidazol-1-yl, isoquinolin-4-yl, 1H-imidazo[4,5-b]pyridin-1-yl, imidazo[1,2-a]pyridin-3-yl, benzo[b]thiophen-3-yl, pyrimidin-5-yl, pyridin-2-yl, pyridin-3-yl, pyridin-4-yl, 1H-imidazol-1-yl, pyrazin-2-yl, pyridazin-4-yl, 1H-pyrrol-2-yl and thiazol-5-yl, wherein said thiophen-2-yl, thiophen-3-yl, furan-3-yl, 1H-benzo[d]imidazol-1-yl, isoquinolin-4-yl, 1H-imidazo[4,5-b]pyridin-1-yl, benzo[b]thiophen-3-yl, pyrimidin-5-yl, pyridin-2-yl, pyridin-3-yl, pyridin-4-yl, 1H-imidazol-1-yl, pyrazin-2-yl, pyridazin-4-yl, 1H-pyrrol-2-yl, or thiazol-5-yl is optionally substituted with from 1 to 3 substituents independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}.

76. The method, use, aryl hydrocarbon receptor antagonist, pharmaceutical composition, or kit of any one of the preceding claims, wherein R₃ is selected from the group consisting of thiophen-3-yl, benzo[b]thiophen-3-yl, pyridin-3-yl, pyrimidin-5-yl, 1H-imidazol-1-yl, 1H-benzo[d]imidazol-1-yl, isoquinolin-4-yl, 1H-imidazo[4,5-b]pyridin-1-yl, and imidazo[1,2-a]pyridin-3-yl, wherein said thiophen-3-yl, benzo[b]thiophen-3-yl, pyridin-3-yl, pyrimidin-5-yl, 1H-imidazol-1-yl, 1H-benzo[d]imidazol-1-yl, isoquinolin-4-yl, 1H-imidazo[4,5-b]pyridin-1-yl, or imidazo[1,2-a]pyridin-3-yl is optionally substituted with from 1 to 3 substituents independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}.

77. The method, use, aryl hydrocarbon receptor antagonist, pharmaceutical composition, or kit of any one of the preceding claims, wherein R₃ is pyridin-3-yl, wherein said pyridin-3-yl is optionally substituted at C5 with a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₈ₐ, —S(O)₀₋₂R₁₁ₐ, —C(O)OR₁₁ₐ, and —C(O)NR₁₁ₐR_{11b}.

78. The method, use, aryl hydrocarbon receptor antagonist, pharmaceutical composition, or kit of any one of the preceding claims, wherein said pyridin-3-yl is substituted at C5 with a substituent selected from the group consisting of ethoxycarbonyl, methoxy, cyano, methyl, methylsulfonyl, fluoro, chloro, trifluoromethyl, ethynyl, and cyclopropyl.

79. The method, use, aryl hydrocarbon receptor antagonist, pharmaceutical composition, or kit of any one of the preceding claims, wherein R₃ is imidazo[1,2-a]pyridin-3-yl, wherein said imidazo[1,2-a]pyridin-3-yl is optionally substituted with a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

80. The method, use, aryl hydrocarbon receptor antagonist, pharmaceutical composition, or kit of any one of the preceding claims, wherein R₃ is benzo[b]thiophen-3-yl, wherein said benzo[b]thiophen-3-yl is optionally substituted with a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

81. The method, use, aryl hydrocarbon receptor antagonist, pharmaceutical composition, or kit of any one of the preceding claims, wherein R₃ is 1H-imidazo[4,5-b]pyridin-1-yl, wherein said 1H-imidazo[4,5-b]pyridin-1-yl is optionally substituted with a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

82. The method, use, aryl hydrocarbon receptor antagonist, pharmaceutical composition, or kit of any one of the preceding claims, wherein R₃ is isoquinolin-4-yl, wherein said isoquinolin-4-yl is optionally substituted with a substituent selected from the group consisting of C1-4 alkyl, halo, halo-substituted-C1-4 alkyl, C1-4 alkenyl, C1-4 alkynyl, C2-8 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

83. The method, use, aryl hydrocarbon receptor antagonist, pharmaceutical composition, or kit of any one of the preceding claims, wherein RA is hydrogen.

84. The method, use, aryl hydrocarbon receptor antagonist, pharmaceutical composition, or kit of any one of the preceding claims, wherein R₃ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein said C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4alkyl.

85. The method, use, aryl hydrocarbon receptor antagonist, pharmaceutical composition, or kit of any one of the preceding claims, wherein R₅ is selected from the group consisting of isopropyl, methyl, ethyl, prop-1-en-2-yl, isobutyl, cyclohexyl, sec-butyl, (S)-sec-butyl, (R)-sec-butyl, 1-hydroxypropan-2-yl, (S)-1-hydroxypropan-2-yl, (R)-1-hydroxypropan-2-yl, and nonan-2-yl.

86. The method, use, aryl hydrocarbon receptor antagonist, pharmaceutical composition, or kit of any one of the preceding claims, wherein R₅ is selected from the group consisting of (i), (ii), (iii), (iv), and (v) 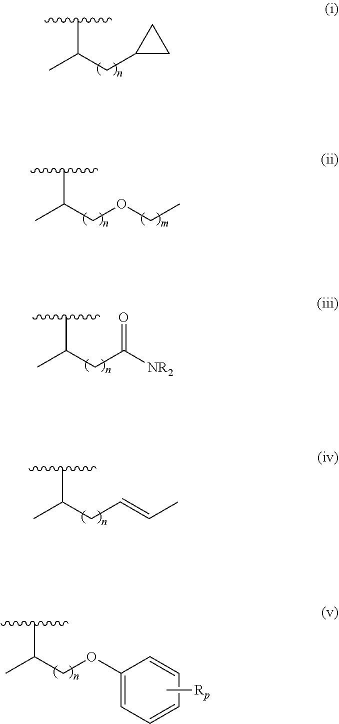
wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

87. The method, use, aryl hydrocarbon receptor antagonist, pharmaceutical composition, or kit of any one of the preceding claims, wherein Re is hydrogen.

88. The method, use, aryl hydrocarbon receptor antagonist, pharmaceutical composition, or kit of any one of the preceding claims, wherein said aryl hydrocarbon receptor antagonist is a compound represented by formula (II-a) 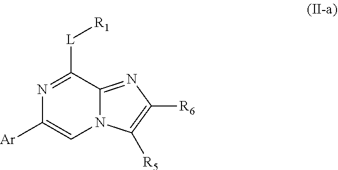
or a pharmaceutically acceptable salt, hydrate, or solvate thereof,
wherein L is selected from the group consisting of —NR₇ₐ(CR₈ₐR_{8b})ₙ—, —O(CR₈ₐR_{8b})ₙ—, —C(O)(CR₈ₐR_{8b})ₙ—, —C(S)(CR₈ₐR_{8b})ₙ—, —S(O)₀₋₂(CR₈ₐR_{8b})ₙ—, —(CR₈ₐR_{8b})ₙ—, —NR₇ₐC(O)(CR₈ₐR_{8b})ₙ—, —NR₇ₐC(S)(CR₈ₐR_{8b})ₙ—, —OC(O)(CR₈ₐR_{8b})ₙ—, —OC(S)(CR₈ₐR_{8b})ₙ—, —C(O)NR₇ₐ(CR₉ₐR₉ₐ)ₙ—, —C(S)NR₇ₐ(CR₈ₐR_{8b})ₙ—, —C(O)O(CR₈ₐR_{8b})ₙ—, —C(S)O(CR₈ₐR_{8b})ₙ—, —S(O)₂NR₇ₐ(CR₈ₐR_{8b})ₙ—, —NR₇ₐS(O)₂(CR₈ₐR_{8b})ₙ—, —NR₇ₐC(O)NR_{7b}(CR₉ₐR_{9b})ₙ—, and —NR₇ₐC(O)O(CR₈ₐR_{8b})ₙ—, wherein R₇ₐ, R_{7b}, R₈ₐ, and R_{8b} are each independently selected from the group consisting of hydrogen and optionally substituted C1-4 alkyl, and each n is independently an integer from 2 to 6;
R₁ is selected from the group consisting of —S(O)₂NR₉ₐR_{9b}, —NR₉ₐC(O)R_{9b}, —NR₉ₐC(S)R₉, —NR₉ₐC(O)NR_{9b}R_{9c}, —C(O)R₉ₐ, —C(S)R₉ₐ, —S(O)₀₋₂R₉, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)CR₉ₐR_{9b}R_{9c}, —OC(S)CR₉ₐR_{9b}R_{9c}, optionally substituted aryl, optionally substituted heteroaryl, optionally substituted cycloalkyl, and optionally substituted heterocycloalkyl, wherein R₉ₐ, R_{9b}, and R_{9c} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;
Ar is selected from the group consisting of optionally substituted monocyclic aryl and heteroaryl;
R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and
R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

89. The method, use, aryl hydrocarbon receptor antagonist, pharmaceutical composition, or kit of any one of the preceding claims, wherein R₁ is selected from the group consisting of phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C1-4 alkyl, C1-4 alkoxy, halo, halo-substituted-C1-4 alkyl, halo-substituted-C1-4 alkoxy, amino, —O(CH₂)₂NR₁₀ₐR_{9b}, —S(O)₂NR₁₀ₐR_{9b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

90. The method, use, aryl hydrocarbon receptor antagonist, pharmaceutical composition, or kit of any one of the preceding claims, wherein said aryl hydrocarbon receptor antagonist is a compound represented by formula (II-b) 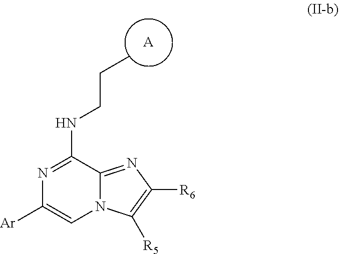
or a pharmaceutically acceptable salt, hydrate, or solvate thereof,
wherein A is an optionally substituted ring system selected from the group consisting of phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b} and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;
Ar is selected from the group consisting of optionally substituted monocyclic aryl and heteroaryl;
R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and
R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

91. The method, use, aryl hydrocarbon receptor antagonist, pharmaceutical composition, or kit of any one of the preceding claims, wherein said aryl hydrocarbon receptor antagonist is a compound represented by formula (II-c) 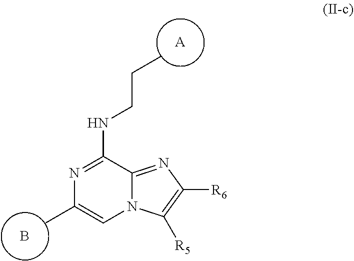
or a pharmaceutically acceptable salt, hydrate, or solvate thereof,
wherein A is an optionally substituted ring system selected from the group consisting of phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C1-4alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;
B is an optionally substituted ring system selected from the group consisting of thiophenyl, furanyl, 1H-benzoimidazolyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl, wherein the thiophenyl, furanyl, 1H-benzoimidazolyl, isoquinolinyl, 1H-imidazopyridinyl, benzothiophenyl, pyrimidinyl, pyridinyl, 1H-imidazolyl, pyrazinyl, pyridazinyl, 1H-pyrrolyl, or thiazolyl is optionally substituted with from 1 to 3 substituents independently selected from the group consisting of cyano, hydroxy, C₁-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl;
R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and
R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

92. The method, use, aryl hydrocarbon receptor antagonist, pharmaceutical composition, or kit of any one of the preceding claims, wherein said aryl hydrocarbon receptor antagonist is a compound represented by formula (II-d) 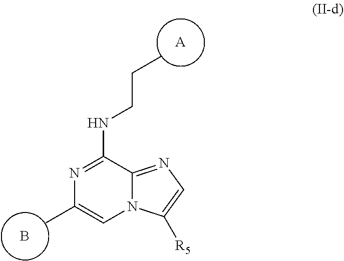
or a pharmaceutically acceptable salt, hydrate, or solvate thereof,
wherein A is an optionally substituted ring system selected from the group consisting of phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b} and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;
B is an optionally substituted ring system selected from the group consisting of thiophenyl, furanyl, 1H-benzoimidazolyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl, wherein the thiophenyl, furanyl, 1H-benzoimidazolyl, isoquinolinyl, 1H-imidazopyridinyl, benzothiophenyl, pyrimidinyl, pyridinyl, 1H-imidazolyl, pyrazinyl, pyridazinyl, 1H-pyrrolyl, or thiazolyl is optionally substituted with from 1 to 3 substituents independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and
R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl.

93. The method, use, aryl hydrocarbon receptor antagonist, pharmaceutical composition, or kit of any one of the preceding claims, wherein said aryl hydrocarbon receptor antagonist is a compound represented by formula (II-e) 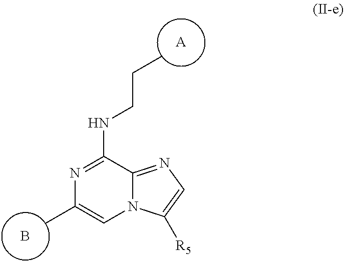
or a pharmaceutically acceptable salt, hydrate, or solvate thereof,
wherein A is an optionally substituted ring system selected from the group consisting of phenyl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, and 2-oxo-2,3-dihydro-1H-benzo[d]imidazol-5-yl, wherein the phenyl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, or 2-oxo-2,3-dihydro-1H-benzo[d]imidazol-5-yl is optionally substituted with from 1 to 3 substituents independently selected from the group consisting of cyano, hydroxy, C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;
B is an optionally substituted ring system selected from the group consisting of thiophen-2-yl, thiophen-3-yl, furan-3-yl, 1H-benzo[d]imidazol-1-yl, isoquinolin-4-yl, 1H-imidazo[4,5-b]pyridin-1-yl, imidazo[1,2-a]pyridin-3-yl, benzo[b]thiophen-3-yl, pyrimidin-5-yl, pyridin-2-yl, pyridin-3-yl, pyridin-4-yl, 1H-imidazol-1-yl, pyrazin-2-yl, pyridazin-4-yl, 1H-pyrrol-2-yl and thiazol-5-yl, wherein the thiophen-2-yl, thiophen-3-yl, furan-3-yl, 1H-benzo[d]imidazol-1-yl, isoquinolin-4-yl, 1H-imidazo[4,5-b]pyridin-1-yl, benzo[b]thiophen-3-yl, pyrimidin-5-yl, pyridin-2-yl, pyridin-3-yl, pyridin-4-yl, 1H-imidazol-1-yl, pyrazin-2-yl, pyridazin-4-yl, 1H-pyrrol-2-yl, or thiazol-5-yl is optionally substituted with from 1 to 3 substituents independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and
R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4alkyl, or R₅ is selected from the group consisting of (i), (ii), (iii), (iv), and (v) 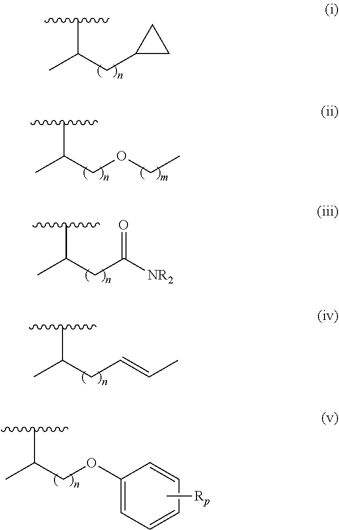
wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

94. The method, use, aryl hydrocarbon receptor antagonist, pharmaceutical composition, or kit of any one of the preceding claims, wherein said aryl hydrocarbon receptor antagonist is a compound represented by formula (II-f) 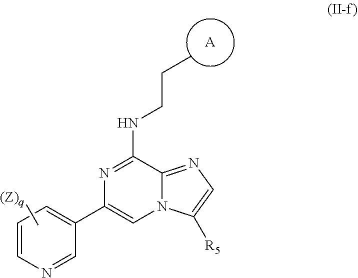
or a pharmaceutically acceptable salt, hydrate, or solvate thereof,
wherein A is an optionally substituted ring system selected from the group consisting of phenol-4-yl and 1H-indol-3-yl;
q is an integer from 0 to 4;
each Z is independently a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and
R₅ is selected from the group consisting of isopropyl, methyl, ethyl, prop-1-en-2-yl, isobutyl, cyclohexyl, sec-butyl, (S)-sec-butyl, (R)-sec-butyl, 1-hydroxypropan-2-yl, (S)-1-hydroxypropan-2-yl, (R)-1-hydroxypropan-2-yl, and nonan-2-yl, or R₅ is selected from the group consisting of (i), (ii), (iii), (iv), and (v) 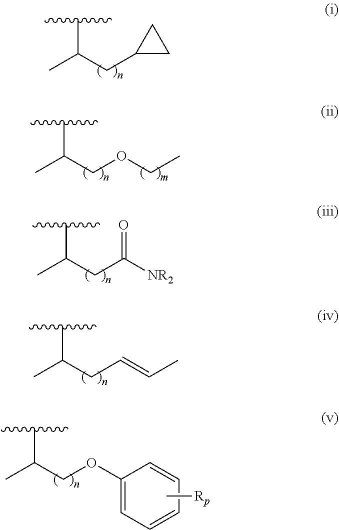
wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

95. The method, use, aryl hydrocarbon receptor antagonist, pharmaceutical composition, or kit of any one of the preceding claims, wherein said aryl hydrocarbon receptor antagonist is a compound represented by formula (II-g) 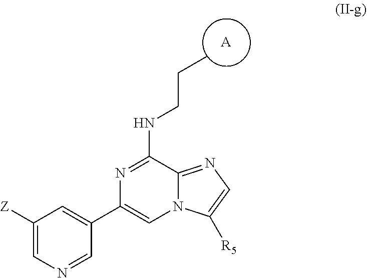
or a pharmaceutically acceptable salt, hydrate, or solvate thereof,
wherein A is an optionally substituted ring system selected from the group consisting of phenol-4-yl and 1H-indol-3-yl;
Z is a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and
R₅ is selected from the group consisting of isopropyl, methyl, ethyl, prop-1-en-2-yl, isobutyl, cyclohexyl, sec-butyl, (S)-sec-butyl, (R)-sec-butyl, 1-hydroxypropan-2-yl, (S)-1-hydroxypropan-2-yl, (R)-1-hydroxypropan-2-yl, and nonan-2-yl, or R₅ is selected from the group consisting of (i), (ii), (iii), (lv), and (v) 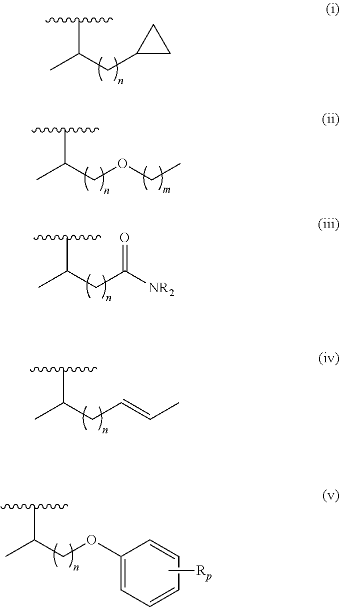
wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄alkyl.

96. The method, use, aryl hydrocarbon receptor antagonist, pharmaceutical composition, or kit of any one of the preceding claims, wherein said aryl hydrocarbon receptor antagonist is a compound represented by formula (II-h) 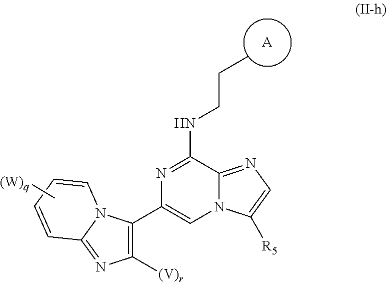
or a pharmaceutically acceptable salt, hydrate, or solvate thereof,
wherein A is an optionally substituted ring system selected from the group consisting of phenol-4-yl and 1H-indol-3-yl;
q is an integer from 0 to 4;
r is 0 or 1;
W and V are each independently a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄alkyl; and
R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4alkyl, or R₅ is selected from the group consisting of (i), (ii), (iii), (iv), and (v) 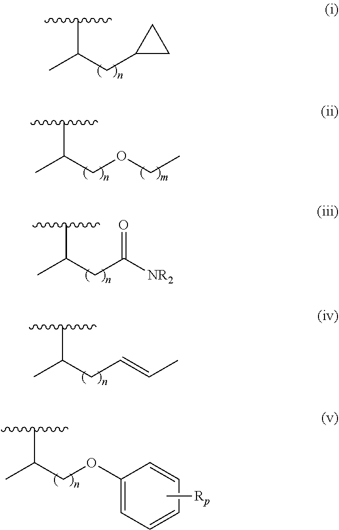
wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

97. The method, use, aryl hydrocarbon receptor antagonist, pharmaceutical composition, or kit of any one of the preceding claims, wherein said aryl hydrocarbon receptor antagonist is a compound represented by formula (II-i) 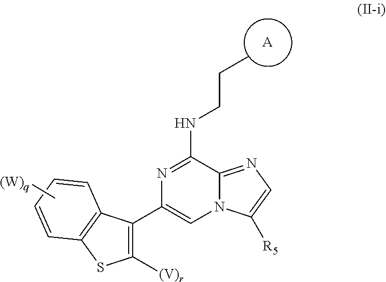
or a pharmaceutically acceptable salt, hydrate, or solvate thereof,
wherein A is an optionally substituted ring system selected from the group consisting of phenol-4-yl and 1H-indol-3-yl;
q is an integer from 0 to 4;
r is 0 or 1;
W and V are each independently a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄alkyl; and
R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-4-yl)-1H-1,2,3-triazol-4-yl)ethyl is optionally substituted with from 1 to 3 substituents independently selected from the group consisting of hydroxy, C1-4 alkyl, and halo-substituted-C1-4alkyl, or R₅ is selected from the group consisting of (i), (ii), (ii), (iv), and (v) 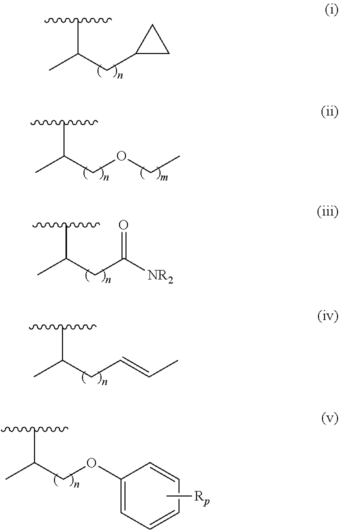
wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

98. The method, use, aryl hydrocarbon receptor antagonist, pharmaceutical composition, or kit of any one of the preceding claims, wherein said aryl hydrocarbon receptor antagonist is a compound represented by formula (II-j) 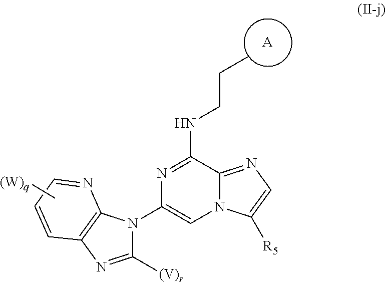
or a pharmaceutically acceptable salt, hydrate, or solvate thereof,
wherein A is an optionally substituted ring system selected from the group consisting of phenol-4-yl and 1H-indol-3-yl;
q is an integer from 0 to 4;
r is 0 or 1;
W and V are each independently a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄alkyl; and
R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4alkyl, or R₅ is selected from the group consisting of (i), (ii), (iii), (iv), and (v) 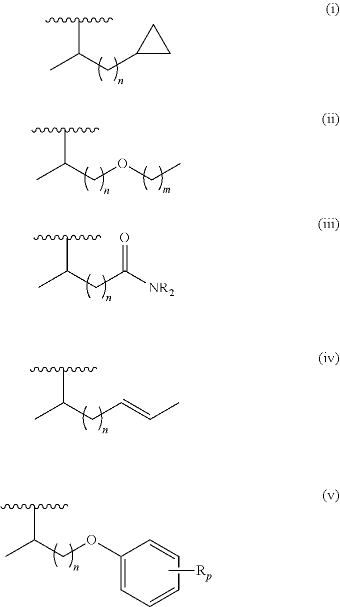
wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

99. The method, use, aryl hydrocarbon receptor antagonist, pharmaceutical composition, or kit of any one of the preceding claims, wherein said aryl hydrocarbon receptor antagonist is a compound represented by formula (II-k) 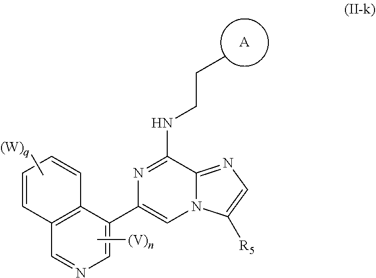
or a pharmaceutically acceptable salt, hydrate, or solvate thereof,
wherein A is an optionally substituted ring system selected from the group consisting of phenol-4-yl and 1H-indol-3-yl;
q is an integer from 0 to 4;
r is 0 or 1;
W and V are each independently a substituent selected from the group consisting of C1-4 alkyl, halo, halo-substituted-C1-4 alkyl, C1-4 alkenyl, C1-4 alkynyl, C2-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄alkyl; and
R₃ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4alkyl, or R₅ is selected from the group consisting of (i), (ii), (iii), (iv), and (v) 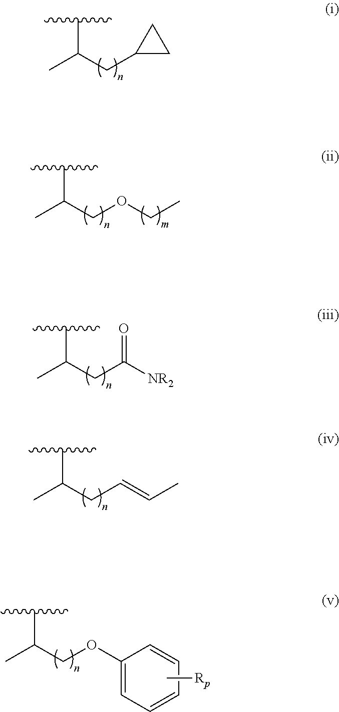
wherein n is an integer from 1 to 6, m is an integer from 0 to 6, p is an integer from 0 to 5, and each R is independently selected from the group consisting of cyano, hydroxy, C1-4 alkyl, C1-4 alkenyl, C1-4 alkynyl, C2-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

100. The method, use, aryl hydrocarbon receptor antagonist, pharmaceutical composition, or kit of any one of the preceding claims, wherein said aryl hydrocarbon receptor antagonist is compound (12) 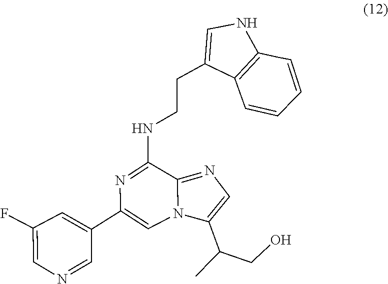 [SMILES: CC(CO)c1cnc2c(NCCc3c[nH]c4c3C=CC=CC4)nc(-c3cncc(F)c3)cn12]
or a salt thereof.

101. The method, use, aryl hydrocarbon receptor antagonist, pharmaceutical composition, or kit of any one of the preceding claims, wherein said aryl hydrocarbon receptor antagonist is compound (13) 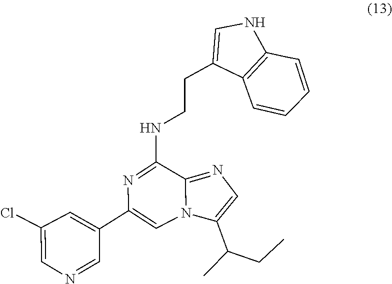 [SMILES: CCC(C)c1cnc2c(NCCc3c[nH]c4c3C=CC=CC4)nc(-c3cncc(Cl)c3)cn12]
or a salt thereof.

102. The method, use, aryl hydrocarbon receptor antagonist, pharmaceutical composition, or kit of any one of the preceding claims, wherein said aryl hydrocarbon receptor antagonist is compound (14) 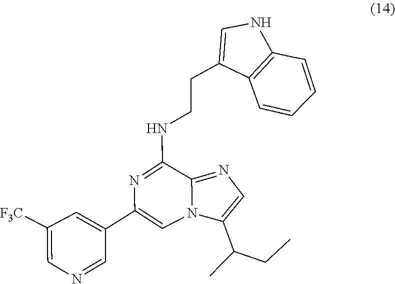 [SMILES: CCC(C)c1cnc2c(NCCc3c[nH]c4c3C=CC=CC4)nc(-c3cncc(C(F)(F)F)c3)cn12]
or a salt thereof.

103. The method, use, aryl hydrocarbon receptor antagonist, pharmaceutical composition, or kit of any one of the preceding claims, wherein said aryl hydrocarbon receptor antagonist is compound (15) 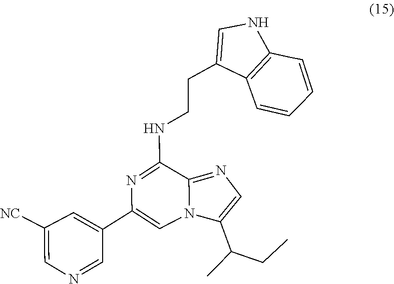 [SMILES: CCC(C)c1cnc2c(NCCc3c[nH]c4c3C=CC=CC4)nc(-c3cncc(C#N)c3)cn12]
or a salt thereof.

104. The method, use, aryl hydrocarbon receptor antagonist, pharmaceutical composition, or kit of any one of the preceding claims, wherein said aryl hydrocarbon receptor antagonist is compound (16) 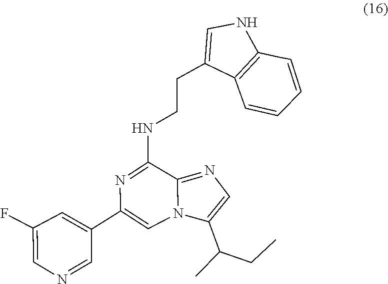 [SMILES: CCC(C)c1cnc2c(NCCc3c[nH]c4c3C=CC=CC4)nc(-c3cncc(F)c3)cn12]
or a salt thereof.

105. The method, use, aryl hydrocarbon receptor antagonist, pharmaceutical composition, or kit of any one of the preceding claims, wherein said aryl hydrocarbon receptor antagonist is compound (17) 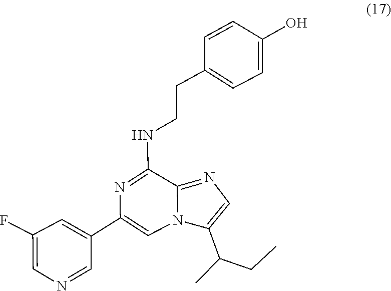 [SMILES: CCC(C)c1cnc2c(NCCc3ccc(O)cc3)nc(-c3cncc(F)c3)cn12]
or a salt thereof.

106. The method, use, aryl hydrocarbon receptor antagonist, pharmaceutical composition, or kit of any one of the preceding claims, wherein said aryl hydrocarbon receptor antagonist is compound (18) 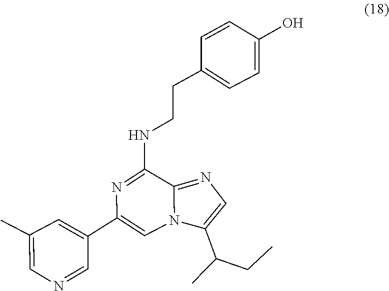 [SMILES: CCC(C)c1cnc2c(NCCc3ccc(O)cc3)nc(-c3cncc(C)c3)cn12]
or a salt thereof.

107. The method, use, aryl hydrocarbon receptor antagonist, pharmaceutical composition, or kit of any one of the preceding claims, wherein said aryl hydrocarbon receptor antagonist is compound (19) 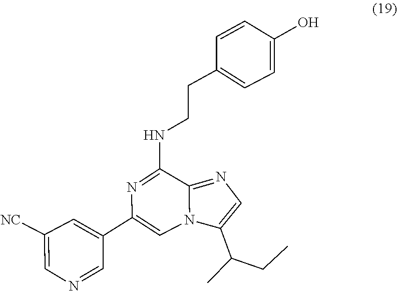 [SMILES: CCC(C)c1cnc2c(NCCc3ccc(O)cc3)nc(-c3cncc(C#N)c3)cn12]
or a salt thereof.

108. The method, use, aryl hydrocarbon receptor antagonist, pharmaceutical composition, or kit of any one of the preceding claims, wherein said aryl hydrocarbon receptor antagonist is compound (20) 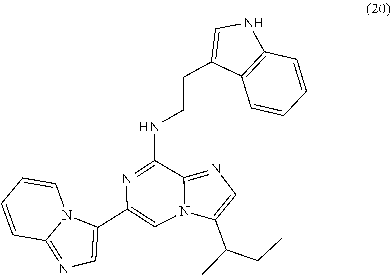 [SMILES: CCC(C)c1cnc2c(NCCc3c[nH]c4ccccc34)nc(-c3cnc4ccccn34)cn12]
or a salt thereof.

109. The method, use, aryl hydrocarbon receptor antagonist, pharmaceutical composition, or kit of any one of the preceding claims, wherein said aryl hydrocarbon receptor antagonist is compound (21) 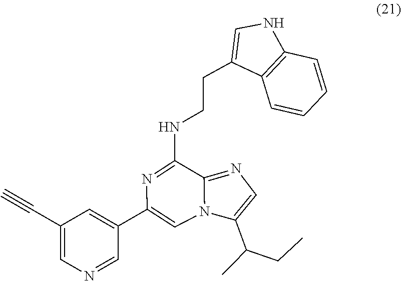 [SMILES: C#Cc1cncc(-c2cn3c(C(C)CC)cnc3c(NCCc3c[nH]c4ccccc34)n2)c1]
or a salt thereof.

110. The method, use, aryl hydrocarbon receptor antagonist, pharmaceutical composition, or kit of any one of the preceding claims, wherein said aryl hydrocarbon receptor antagonist is compound (22) 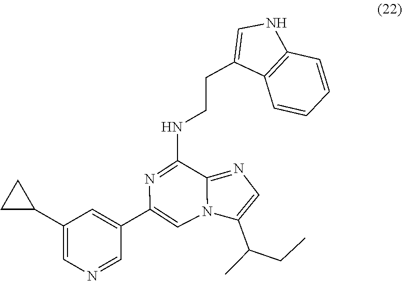 [SMILES: CCC(C)c1cnc2c(NCCc3c[nH]c4ccccc34)nc(-c3cncc(C4CC4)c3)cn12]
or a salt thereof.

111. The method, use, aryl hydrocarbon receptor antagonist, pharmaceutical composition, or kit of any one of the preceding claims, wherein said aryl hydrocarbon receptor antagonist is compound (24) 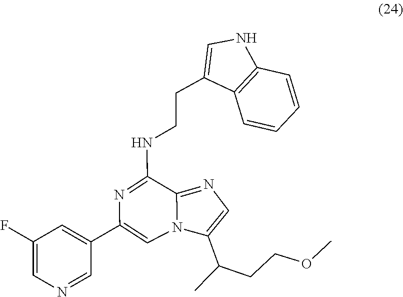 [SMILES: COCCC(C)c1cnc2c(NCCc3c[nH]c4ccccc34)nc(-c3cncc(F)c3)cn12]
or a salt thereof.

112. The method, use, aryl hydrocarbon receptor antagonist, pharmaceutical composition, or kit of any one of the preceding claims, wherein said aryl hydrocarbon receptor antagonist is compound (27) 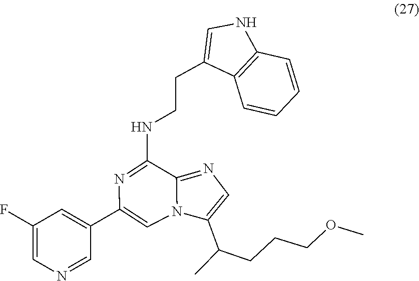 [SMILES: COCCCC(C)c1cnc2c(NCCc3c[nH]c4ccccc34)nc(-c3cncc(F)c3)cn12]
or a salt thereof.

113. The method, use, aryl hydrocarbon receptor antagonist, pharmaceutical composition, or kit of any one of the preceding claims, wherein said aryl hydrocarbon receptor antagonist is compound (28) 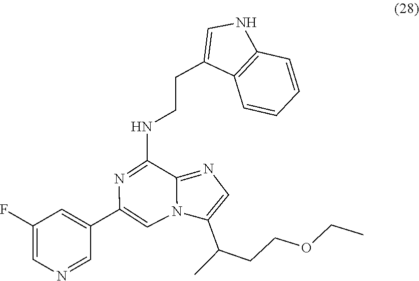 [SMILES: CCOCCC(C)c1cnc2c(NCCc3c[nH]c4ccccc34)nc(-c3cncc(F)c3)cn12]
or a salt thereof.
